(19) United States
(12) Patent Application Publication
(10) Pub. No.: US 2025/0051779 A1
(43) Pub. Date: Feb. 13, 2025

(54) INHIBIN SUBUNIT BETA E (INHBE) MODULATOR COMPOSITIONS AND METHODS OF USE THEREOF

(71) Applicant: Alnylam Pharmaceuticals, Inc., Cambridge, MA (US)

(72) Inventor: Aimee M. Deaton, Somerville, MA (US)

(21) Appl. No.: 18/607,663

(22) Filed: Mar. 18, 2024

Related U.S. Application Data
(63) Continuation of application No. PCT/US2022/043948, filed on Sep. 19, 2022.
(60) Provisional application No. 63/246,084, filed on Sep. 20, 2021.

(51) Int. Cl. C12N 15/113

(52) U.S. Cl. CPC C12N 15/1136; C12N 2310/11; C12N 2310/321; C12N 2310/322; C12N 2310/3341; C12N 2310/341

(57) ABSTRACT

The present invention relates to modulators, e.g., double stranded RNA (dsRNA) agents, antisense polynucleotide agents, antibodies, guideRNAs that effect ADAR editing, or guideRNAs that effect CRISPR editing, that modulate, e.g., inhibit, the expression and/or activity of inhibin subunit beta E (INHBE). The invention also relates to methods of using such modulators to inhibit expression and/or activity of INHBE and to methods of preventing and treating an INHBE-associated disorder, e.g., metabolic disorder, e.g., metabolic syndrome, in a subject.

### RELATED APPLICATIONS

This application is a 35 § U.S.C. 111(a) continuation application which claims the benefit of priority to International Application No. PCT/US2022/043948, filed on Sep. 19, 2022, which, in turn, claims the benefit of priority to U.S. Provisional Application No. 63/246,084, filed on Sep. 20, 2021. The entire contents of each of the foregoing applications are incorporated herein by reference.

This application is related to U.S. Provisional Application No. 63/223,995, filed on Jul. 21, 2021, U.S. Provisional Application No. 63/278,126, filed on Nov. 11, 2021, U.S. Provisional Application No. 63/285,143, filed on Dec. 2, 2021, U.S. Provisional Application No. 63/287,578, filed on Dec. 9, 2021, U.S. Provisional Application No. 63/321,799, filed on Mar. 21, 2022, U.S. Provisional Application No. 63/323,543, filed on Mar. 25, 2022, and PCT Application No. PCT/US2022/037658, filed on Jul. 20, 2022. The entire contents of each of the foregoing applications are incorporated herein by reference.

### SEQUENCE LISTING

The instant application contains a Sequence Listing which has been submitted electronically in XML file format via Patent Center and is hereby incorporated by reference in its entirety. Said XML copy, created on Apr. 19, 2024, is named 121301_17002_SL.xml and is 5,341,801 bytes in size.

### BACKGROUND OF THE INVENTION

With the successful conquest of many infectious diseases in most of the world, non-communicable diseases, metabolic disorders in particular, have become a major health hazard of the modern world. The increase in consumption of high calorie-low fiber fast food and the decrease in physical activity due to mechanized transportations and sedentary lifestyle have resulted in the spread of metabolic disorders such as metabolic syndrome, type 2 diabetes, hypertension, cardiovascular diseases, stroke, and other disabilities. Indeed, the occurences of subjects with a metabolic disorder, such as, metabolic syndrome, who have a number of health conditions placing them at higher risk for heart disease, diabetes, stroke, and other diseases have increased in the recent years.

Current treatments for disorders of lipid metabolism include lifestyle changes, dieting, exercise and treatment with agents, such as lipid lowering agents, e.g., statins, and other drugs. However, these therapies and treatments are often limited by compliance, are not always effective, result in side effects, and result in drug-drug interactions. Accordingly, there is a need in the art for alternative treatments for subjects having metabolic disorders.

Inhibin subunit beta E (INHBE) is a member of the transforming growth factor-β (TGF-β) family. Predominantly expressed in the liver, INHBE is a hepatokine which has been shown to positively correlate with insulin resistance and body mass index in humans. Quantitative real time-PCR analysis also showed an increase in INHBE gene expression in liver samples from insulin-resistant human subjects. In addition, Inhbe gene expression was shown to be increased in the livers of an art-recognized animal model of a metabolic disorder, i.e., type 2 diabetes, the db/db mouse model. Inhibition of Inhbe expression in db/db mice was demonstrated to suppress body weight gain which was attributable to diminished fat rather than lean mass.

As indicated above, there is an unmet need for effective treatments for metablic disorders, such as metablic syndrome and related diseases, e.g., diabetes, hypertension, and cardiovascular disease, such as an agent that can selectively and efficiently modulate, i.e., inhibit INHBE expression and/or activity.

### SUMMARY OF THE INVENTION

The present invention provides inter alia a modulator that modulates, i.e., inhibits, the expression and/or activity of inhibin subunit beta E (INHBE) for treating an INHBE-associated disorder, e.g. a metabolic disorder, e.g., metabolic syndrome.

In one aspect, the present invention provides a modulator of inhibin subunit beta E (INHBE). The modulator may be an oligonucleotide that targets INHBE, such as a double stranded ribonucleic acid (dsRNA) or an antisense polynucleotide agent; an antibody, or antigen-binding fragment thereof, that specifically binds INHBE, such as a monoclonal anti-INHBE antibody, or antigen-binding fragment thereof; a small molecule; a guideRNA that effects ADAR editing, such as a guideRNA that includes a stem loop structure that binds the ADAR enzyme; or a guideRNA that effects CRISPR editing.

In one embodiment, the antisense polynucleotide agent comprises 4 to 50 contiguous nucleotides, wherein at least one of the contiguous nucleotides is a modified nucleotide, and wherein the nucleotide sequence of the agent is 80% complementary over its entire length to the equivalent region of the nucleotide sequence of any one of SEQ ID NOs:1, 2, 4, 6, 8, or 10.

In one embodiment, the equivalent region is any one of the target regions of SEQ ID NO:1 provided in Table 4.

In one embodiment, the antisense polynucleotide agent comprises at least 8 contiguous nucleotides differing by no more than 3 nucleotides from any one of the nucleotide sequences listed in Table 3.

In one embodiment, substantially all of the nucleotides of the antisense polynucleotide agent are modified nucleotides.

In one embodiment, all of the nucleotides of the antisense polynucleotide agent are modified nucleotides.

In one embodiment, the antisense polynucleotide agent is 10 to 40 nucleotides in length.

In one embodiment, the antisense polynucleotide agent is 10 to 30 nucleotides in length.

In one embodiment, the antisense polynucleotide agent is 18 to 30 nucleotides in length.

In one embodiment, the antisense polynucleotide agent is 10 to 24 nucleotides in length.

In one embodiment, the antisense polynucleotide agent is 18 to 24 nucleotides in length.

In one embodiment, the antisense polynucleotide agent is 14 to 20 nucleotides in length.

In one embodiment, the antisense polynucleotide agent is 14 nucleotides in length.

In one embodiment, the antisense polynucleotide agent is 20 nucleotides in length.

In one embodiment, the modified nucleotide comprises a modified sugar moiety selected from the group consisting of a 2′-O-methoxyethyl modified sugar moiety, a 2′-methoxy modified sugar moiety, a 2′-O-alkyl modified sugar moiety, and a bicyclic sugar moiety.

In one embodiment, the bicyclic sugar moiety has a (—CH2-)n group forming a bridge between the 2′ oxygen and the 4′ carbon atoms of the sugar ring, wherein n is 1 or 2 and wherein R is H, CH3 or CH3OCH3.

In one embodiment, the modified nucleotide is a 5-methylcytosine.

In one embodiment, the modified nucleotide comprises a modified internucleoside linkage.

In one embodiment, the modified internucleoside linkage is a phosphorothioate internucleoside linkage.

In one embodiment, the modulator comprises a plurality of 2′-deoxynucleotides flanked on each side by at least one nucleotide having a modified sugar moiety.

In one embodiment, the antisense polynucleotide agent is a gapmer comprising a gap segment comprised of linked 2′-deoxynucleotides positioned between a 5′ and a 3′ wing segment.

In one embodiment, the modified sugar moiety is selected from the group consisting of a 2′-O-methoxyethyl modified sugar moiety, a 2′-methoxy modified sugar moiety, a 2′-O-alkyl modified sugar moiety, and a bicyclic sugar moiety.

In one embodiment, the 5′-wing segment is 1 to 6 nucleotides in length.

In one embodiment, the 3′-wing segment is 1 to 6 nucleotides in length.

In one embodiment, the gap segment is 5 to 14 nucleotides in length.

In one embodiment, the 5′-wing segment is 2 nucleotides in length.

In one embodiment, the 3′-wing segment is 2 nucleotides in length.

In one embodiment, the 5′-wing segment is 3 nucleotides in length.

In one embodiment, the 3′-wing segment is 3 nucleotides in length.

In one embodiment, the 5′-wing segment is 4 nucleotides in length.

In one embodiment, the 3′-wing segment is 4 nucleotides in length.

In one embodiment, the 5′-wing segment is 5 nucleotides in length.

In one embodiment, the 3′-wing segment is 5 nucleotides in length.

In one embodiment, the gap segment is 10 nucleotides in length.

In one embodiment, the antisense polynucleotide agent comprises a gap segment consisting of linked deoxynucleotides; a 5′-wing segment consisting of linked nucleotides; a 3′-wing segment consisting of linked nucleotides; wherein the gap segment is positioned between the 5′-wing segment and the 3′-wing segment and wherein each nucleotide of each wing segment comprises a modified sugar.

In one embodiment, the gap segment is ten 2′-deoxynucleotides in length and each of the wing segments is five nucleotides in length.

In one embodiment, the gap segment is ten 2′-deoxynucleotides in length and each of the wing segments is four nucleotides in length.

In one embodiment, the gap segment is ten 2′-deoxynucleotides in length and each of the wing segments is three nucleotides in length.

In one embodiment, the gap segment is ten 2′-deoxynucleotides in length and each of the wing segments is two nucleotides in length.

In one embodiment, the modified sugar moiety is selected from the group consisting of a 2′-O-methoxyethyl modified sugar moiety, a 2′-methoxy modified sugar moiety, a 2′-O-alkyl modified sugar moiety, and a bicyclic sugar moiety.

In one embodiment, all of the nucleotides comprise a modified internucleoside linkage.

In one embodiment, the modulator further comprises a ligand.

In one embodiment, the modulator is conjugated to the ligand at the 3′-terminus.

In one embodiment, the ligand is an N-acetylgalactosamine (GalNAc) derivative.

In one embodiment, the ligand is

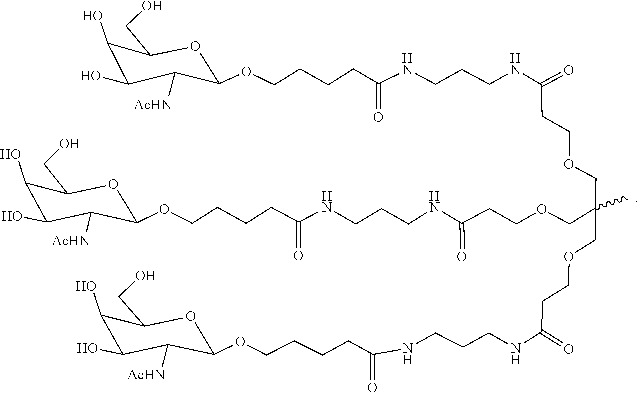 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

The present invention also provides cells containing any of the modulators of the invention and pharmaceutical compositions comprising any of themodulators of the invention.

The pharmaceutical composition of the invention may include a modulator in an unbuffered solution, e.g., saline or water, or the pharmaceutical composition of the invention may include the modulator in a buffer solution, e.g., a buffer solution comprising acetate, citrate, prolamine, carbonate, or phosphate or any combination thereof; or phosphate buffered saline (PBS). In some embodiments, the pharmaceutical compositions comprises a modulator and a lipid formulation, e.g., the lipid formulation comprises a LNP or the lipid formulation comprises a MC3.

In one aspect, the present invention provides a method of inhibiting expression and/or activity of inhibin subunit beta E (INHBE) in a cell. The method includes contacting the cell with any of the modulators of the invention or any of the pharmaceutical compositions of the invention, thereby inhibiting expression and/or activity of the INHBE gene in the cell.

In one embodiment, the cell is within a subject, e.g., a human subject, e.g., a subject having a metabolic disorder, such as diabetes, or cardiovascular disease, such as hypertension

In certain embodiments, the INHBE expression and/or activity is inhibited by at least about 30%, 40%, 50%, 60%, 70%, 80%, 90%, or 95%. In one embodiment, inhibiting expression and/or activity of INHBE decreases INHBE protein level in serum of the subject by at least 30%, 40%, 50%, 60%, 70%, 80%, 90%, or 95%.

In one aspect, the present invention provides a method of treating a subject having a disorder that would benefit from reduction in inhibin subunit beta E (INHBE) expression and/or activity. The method includes administering to the subject a therapeutically effective amount of any of the modulators of the invention or any of the pharmaceutical compositions of the invention, thereby treating the subject having the disorder that would benefit from reduction in INHBE expression.

In another aspect, the present invention provides a method of preventing at least one symptom in a subject having a disorder that would benefit from reduction in inhibin subunit beta E (INHBE) expression and/or activity. The method includes administering to the subject a prophylactically effective amount of any of the modulators of the invention or any of the pharmaceutical compositions of the invention, thereby preventing at least one symptom in the subject having the disorder that would benefit from reduction in INHBE expression.

In one embodiment, administration of a therapeutically or prophylactically effective amount decreases the waist-to-hip ratio adjusted for body mass index in the subject.

In certain embodiments, the disorder is a metabolic disorder, e.g. metabolic syndrome, a disorder of carbohydrates, e.g., type II diabetes, pre-diabetes, a lipid metabolism disorder, e.g., a hyperlipidemia, hypertension, a cardiovascular disease, a disorders of body weight.

In some embodiments, the INHBE-associated disorder is metabolic syndrome.

In some embodiments, the INHBE-associated disorder is cardiovascular disease.

In some embodiments, the INHBE-associated disorder is hypertension.

In certain embodiments, administration of the modulator to the subject causes a decrease INHBE protein accumulation in the subject.

In a further aspect, the present invention also provides methods of inhibiting the expression and/or activity of INHBE in a subject. The methods include administering to the subject a therapeutically effective amount of any of the modulators provided herein, thereby inhibiting the expression and/or activity of INHBE in the subject.

In one embodiment, the subject is human.

In one embodiment, the modulator is administered to the subject at a dose of about 0.01 mg/kg to about 50 mg/kg.

In one embodiment, the modulator is administered to the subject subcutaneously.

In one embodiment, the methods of the invention include further determining the level of INHBE in a sample(s) from the subject.

In one embodiment, the level of INHBE in the subject sample(s) is an INHBE protein level in a blood or serum or liver tissue sample(s).

In certain embodiments, the methods of the invention further comprise administering to the subject an additional therapeutic agent.

In certain embodiments, the additional therapeutic agent is selected from the group consisting of insulin, a glucagon-like peptide 1 agonist, a sulfonylurea, a seglitinide, a biguanide, a thiazolidinedione, an alpha-glucosidase inhibitor, an SGLT2 inhibitor, a DPP-4 inhibitor, an HMG-CoA reductase inhibitor, a statin, and a combination of any of the foregoing.

The present invention also provides kits comprising any of the modulators of the invention or any of the pharmaceutical compositions of the invention, and optionally, instructions for use. In one embodiment, the invention provides a kit for performing a method of inhibiting expression and/or activity of INHBE in a cell by contacting a cell with a modulator of the invention in an amount effective to inhibit expression and/or activity of INHBE in the cell. The kit comprises a modulator and instructions for use and, optionally, means for administering the modulator to a subject.

### DETAILED DESCRIPTION OF THE INVENTION

The present invention provides compositions comprising a modulator, i.e., inhibitor, of inhibin subunit beta E (INHBE) gene for treating an inhibin subunit beta E (INHBE)-associated disorder, e.g., a metabolic disorder, e.g. metabolic syndrome, a disorder of carbohydrates, e.g., type II diabetes, pre-diabetes, a lipid metabolism disorder, e.g., a hyperlipidemia, hypertension, a cardiovascular disease, a disorders of body weight.

The following detailed description discloses how to make and use compositions containing modulators to inhibit the expression and/or activity of INHBE as well as compositions, uses, and methods for treating subjects that would benefit from inhibition and/or reduction of the expression and/or activity of INHBE, e.g., subjects susceptible to or diagnosed with an INHBE-associated disorder.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

The term “at least”, “no less than”, or “or more” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 19 nucleotides of a 21 nucleotide nucleic acid molecule” means that 19, 20, or 21 nucleotides have the indicated property. When at least is present before a series of numbers or a range, it is understood that “at least” can modify each of the numbers in the series or range.

As used herein, “no more than” or “or less” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

As used herein, methods of detection can include determination that the amount of analyte present is below the level of detection of the method.

In the event of a conflict between an indicated target site and the nucleotide sequence for a sense or antisense strand, the indicated sequence takes precedence.

In the event of a conflict between a sequence and its indicated site on a transcript or other sequence, the nucleotide sequence recited in the specification takes precedence.

As used herein, a “modulator” is a molecule that decreases or increases the expression and/or activity of INHBE.

As used herein, “inhibin subunit beta E,” used interchangeably with the terms “INHBE,” refers to a growth factor that belongs to the transforming growth factor-β (TGF-β) family. INHBE mRNA is predominantly expressed in the liver (Fang J. et al. Biochemical & Biophysical Res. Comm. 1997; 231(3):655-61), and INHBE is involved in the regulation of liver cell growth and differentiation (Chabicovsky M. et al. Endocrinology. 2003; 144(8):3497-504). INHBE is also known as inhibin beta E chain, activin beta E, inhibin beta E subunit, inhibin beta E, and MGC4638.

The sequence of a human INHBE mRNA transcript can be found at, for example, GenBank Accession No. GI: 1877089956 (NM 031479.5; SEQ ID NO:1; reverse complement, SEQ ID NO: 2). The sequence of mouse INHBE mRNA can be found at, for example, GenBank Accession No. GI: 1061899809 (NM_008382.3; SEQ ID NO:3; reverse complement, SEQ ID NO:4). The sequence of rat INHBE mRNA can be found at, for example, GenBank Accession No. GI: 148747589 (NM_031815.2; SEQ ID NO:5; reverse complement, SEQ ID NO: 6). The predicted sequence of Macaca mulatta INHBE mRNA can be found at, for example, GenBank Accession No. GI: 1622845604 (XM_001115958.3; SEQ ID NO:7; reverse complement, SEQ ID NO:8).

Additional examples of INHBE mRNA sequences are readily available through publicly available databases, e.g., GenBank, UniProt, OMIM, and the Macaca genome project web site.

Further information on INHBE can be found, for example, at www.ncbi.nlm.nih.gov/gene/?term=INHBE.

The entire contents of each of the foregoing GenBank Accession numbers and the Gene database numbers are incorporated herein by reference as of the date of filing this application.

The term INHBE, as used herein, also refers to variations of the INHBE gene including variants provided in the SNP database. Numerous sequence variations within the INHBE gene have been identified and may be found at, for example, NCBI dbSNP and UniProt (see, e.g., www.ncbi.nlm.nih.gov/snp/?term=INHBE, the entire contents of which is incorporated herein by reference as of the date of filing this application.

As used herein, “target sequence” refers to a contiguous portion of the nucleotide sequence of an mRNA molecule formed during the transcription of an INHBE gene, including mRNA that is a product of RNA processing of a primary transcription product.

In one embodiment, the target portion of the sequence will be at least long enough to serve as a substrate for iRNA-directed cleavage at or near that portion of the nucleotide sequence of an mRNA molecule formed during the transcription of an INHBE gene. In another embodiment, the target sequence is a nucleic acid molecule to which an antisense polynucleotide agent of the invention specifically hybridizes

The target sequence may be from about 19-36 nucleotides in length, e.g., about 19-30 nucleotides in length. For example, the target sequence can be about 19-30 nucleotides, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. In certain embodiments, the target sequence is 19-23 nucleotides in length, optionally 21-23 nucleotides in length. Ranges and lengths intermediate to the above recited ranges and lengths are also contemplated to be part of the disclosure.

As used herein, the term “strand comprising a sequence” refers to an oligonucleotide comprising a chain of nucleotides that is described by the sequence referred to using the standard nucleotide nomenclature.

“G,” “C,” “A,” “T,” and “U” each generally stand for a nucleotide that contains guanine, cytosine, adenine, thymidine, and uracil as a base, respectively. However, it will be understood that the term “ribonucleotide” or “nucleotide” can also refer to a modified nucleotide, as further detailed below, or a surrogate replacement moiety (see, e.g., Table 1). The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of oligonucleotides featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The terms “iRNA”, “RNAi agent,” “iRNA agent,”, “RNA interference agent” as used interchangeably herein, refer to an agent that contains RNA as that term is defined herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of an INHBE gene in a cell, e.g., a liver cell within a subject, such as a mammalian subject.

In one embodiment, an RNAi agent of the invention includes a single stranded RNA that interacts with a target RNA sequence, e.g., an INHBE target mRNA sequence, to direct the cleavage of the target RNA. Without wishing to be bound by theory it is believed that long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded RNA (siRNA) generated within a cell and which promotes the formation of a RISC complex to effect silencing of the target gene, i.e., an INHBE gene. Accordingly, the term “siRNA” is also used herein to refer to an iRNA as described above.

In certain embodiments, the RNAi agent may be a single-stranded siRNA (ssRNAi) that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In certain embodiments, an “iRNA” for use in the compositions, uses, and methods of the invention is a double stranded RNA and is referred to herein as a “double stranded RNA agent,” “double stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, i.e., an INHBE gene. In some embodiments of the invention, a double stranded RNA (dsRNA) triggers the degradation of a target RNA, e.g., an mRNA, through a post-transcriptional gene-silencing mechanism referred to herein as RNA interference or RNAi.

In general, the majority of nucleotides of an oligonucleotide of the invention, e.g., each strand of a dsRNA molecule, are ribonucleotides, but as described in detail herein, each or both strands can also include one or more non-ribonucleotides, e.g., a deoxyribonucleotide or a modified nucleotide. In addition, as used in this specification, an “iRNA” or an “antisense polynucleotide agent” may include ribonucleotides with chemical modifications; an iRNA or antisense polynucleotide agent may include substantial modifications at multiple nucleotides. As used herein, the term “modified nucleotide” refers to a nucleotide having, independently, a modified sugar moiety, a modified internucleotide linkage, or modified nucleobase, or any combination thereof. Thus, the term modified nucleotide encompasses substitutions, additions or removal of, e.g., a functional group or atom, to internucleoside linkages, sugar moieties, or nucleobases. The modifications suitable for use in the agents of the invention include all types of modifications disclosed herein or known in the art. Any such modifications, as used in a siRNA type molecule, are encompassed by “iRNA” or “RNAi agent” or “antisense polynucleotide agent” for the purposes of this specification and claims.

In certain embodiments of the instant disclosure, inclusion of a deoxy-nucleotide if present within an RNAi agent or antisense polynucleotide agent can be considered to constitute a modified nucleotide.

The duplex region of an RNAi agent may be of any length that permits specific degradation of a desired target RNA through a RISC pathway, and may range from about 19 to 36 base pairs in length, e.g., about 19-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. In certain embodiments, the duplex region is 19-21 base pairs in length, e.g., 21 base pairs in length. Ranges and lengths intermediate to the above recited ranges and lengths are also contemplated to be part of the disclosure.

The two strands forming the duplex structure of an RNAi molecule may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 2, 3, 4, 5, 6, 7, 8, 9, 10, 20, 23 or more unpaired nucleotides. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

Where the two substantially complementary strands of a dsRNA are comprised by separate RNA molecules, those molecules need not be, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs. In one embodiment of the RNAi agent, at least one strand comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In certain embodiments, an iRNA agent of the invention is a dsRNA, each strand of which comprises 19-23 nucleotides, that interacts with a target RNA sequence, e.g., an INHBE gene, to direct cleavage of the target RNA.

In some embodiments, an iRNA of the invention is a dsRNA of 24-30 nucleotides that interacts with a target RNA sequence, e.g., an INHBE target mRNA sequence, to direct the cleavage of the target RNA.

As used herein, the term “nucleotide overhang” refers to at least one unpaired nucleotide that protrudes from the duplex structure of a double stranded iRNA. For example, when a 3-end of one strand of a dsRNA extends beyond the 5-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5-end, 3-end, or both ends of either an antisense or sense strand of a dsRNA.

In one embodiment, the antisense strand of a dsRNA has a 1-10 nucleotide, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the antisense strand of a dsRNA has a 1-10 nucleotide, e.g., 0-3, 1-3, 2-4, 2-5, 4-10, 5-10, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the antisense strand of a dsRNA has a 1-10 nucleotides, e.g., a 1, 2, 3, 4, 5, 6, 7, 8, 9, or 10 nucleotide, overhang at the 3′-end or the 5′-end. In certain embodiments, the overhang on the sense strand or the antisense strand, or both, can include extended lengths longer than 10 nucleotides, e.g., 1-30 nucleotides, 2-30 nucleotides, 10-30 nucleotides, 10-25 nucleotides, 10-20 nucleotides, or 10-15 nucleotides in length. In certain embodiments, an extended overhang is on the sense strand of the duplex. In certain embodiments, an extended overhang is present on the 3′ end of the sense strand of the duplex. In certain embodiments, an extended overhang is present on the 5′ end of the sense strand of the duplex. In certain embodiments, an extended overhang is on the antisense strand of the duplex. In certain embodiments, an extended overhang is present on the 3′end of the antisense strand of the duplex. In certain embodiments, an extended overhang is present on the 5′end of the antisense strand of the duplex. In certain embodiments, one or more of the nucleotides in the extended overhang is replaced with a nucleoside thiophosphate. In certain embodiments, the overhang includes a self-complementary portion such that the overhang is capable of forming a hairpin structure that is stable under physiological conditions.

“Blunt” or “blunt end” means that there are no unpaired nucleotides at that end of the double stranded RNA agent, i.e., no nucleotide overhang. A “blunt ended” double stranded RNA agent is double stranded over its entire length, i.e., no nucleotide overhang at either end of the molecule. The RNAi agents of the invention include RNAi agents with no nucleotide overhang at one end (i.e., agents with one overhang and one blunt end) or with no nucleotide overhangs at either end. Most often such a molecule will be double-stranded over its entire length.

The term “antisense strand” or “guide strand” refers to the strand of an iRNA, e.g., a dsRNA, which includes a region that is substantially complementary to a target sequence, e.g., an INHBE mRNA.

As used herein, the term “region of complementarity” refers to the region on the antisense strand of a dsRNA agent or the region of an antisense polynucleotide agent that is substantially complementary to a sequence, for example a target sequence, e.g., an INHBE nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, or 3 nucleotides of the 5′- or 3′-end of the iRNA. In some embodiments, a double stranded RNA agent or antisense polynucleotide agent of the invention includes a nucleotide mismatch in the antisense strand. In some embodiments, the antisense strand of the double stranded RNA agent antisense polynucleotide agent of the invention includes no more than 4 mismatches with the target mRNA, e.g., the antisense strand includes 4, 3, 2, 1, or 0 mismatches with the target mRNA. In some embodiments, the antisense strand double stranded RNA agent of the invention includes no more than 4 mismatches with the sense strand, e.g., the antisense strand includes 4, 3, 2, 1, or 0 mismatches with the sense strand. In some embodiments, a double stranded RNA agent of the invention includes a nucleotide mismatch in the sense strand. In some embodiments, the sense strand of the double stranded RNA agent of the invention includes no more than 4 mismatches with the antisense strand, e.g., the sense strand includes 4, 3, 2, 1, or 0 mismatches with the antisense strand. In some embodiments, the nucleotide mismatch is, for example, within 5, 4, 3 nucleotides from the 3′-end of the iRNA. In another embodiment, the nucleotide mismatch is, for example, in the 3′-terminal nucleotide of the iRNA agent. In some embodiments, the mismatch(s) is not in the seed region.

Thus, an RNAi agent or antisense polynucleotide agent as described herein can contain one or more mismatches to the target sequence. In one embodiment, an RNAi agent or antisense polynucleotide agent as described herein contains no more than 3 mismatches (i.e., 3, 2, 1, or 0 mismatches). In one embodiment, an RNAi agent or antisense polynucleotide agent as described herein contains no more than 2 mismatches. In one embodiment, an RNAi agent or antisense polynucleotide agent as described herein contains no more than 1 mismatch. In one embodiment, an RNAi agent or antisense polynucleotide agent as described herein contains 0 mismatches. In certain embodiments, if the antisense strand of the RNAi agent or antisense polynucleotide agent contains mismatches to the target sequence, the mismatch can optionally be restricted to be within the last 5 nucleotides from either the 5′- or 3′-end of the region of complementarity. For example, in such embodiments, for a 23 nucleotide RNAi agent, the strand which is complementary to a region of an INHBE gene, generally does not contain any mismatch within the central 13 nucleotides. The methods described herein or methods known in the art can be used to determine whether an RNAi agent or antisense polynucleotide agent containing a mismatch to a target sequence is effective in inhibiting the expression of an INHBE gene. Consideration of the efficacy of RNAi agents or antisense polynucleotide agent with mismatches in inhibiting expression of an INHBE gene is important, especially if the particular region of complementarity in an INHBE gene is known to have polymorphic sequence variation within the population.

The term “sense strand” or “passenger strand” as used herein, refers to the strand of an iRNA that includes a region that is substantially complementary to a region of the antisense strand as that term is defined herein.

As used herein, “substantially all of the nucleotides are modified” are largely but not wholly modified and can include not more than 5, 4, 3, 2, or 1 unmodified nucleotides.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3, or 2 mismatched base pairs upon hybridization for a duplex up to 30 base pairs, while retaining the ability to hybridize under the conditions most relevant to their ultimate application, e.g., inhibition of gene expression, in vitro or in vivo. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogsteen base pairing.

The terms “complementary,” “fully complementary” and “substantially complementary” herein can be used with respect to the base matching between the sense strand and the antisense strand of a dsRNA, or between two oligonucleotides or polynucleotides, such as the antisense strand of a double stranded RNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding an INHBE gene). For example, a polynucleotide is complementary to at least a part of an INHBE mRNA if the sequence is substantially complementary to a non-interrupted portion of an mRNA encoding an INHBE gene.

Accordingly, in some embodiments, the antisense polynucleotides disclosed herein are fully complementary to the target INHBE sequence. In other embodiments, the antisense polynucleotides disclosed herein are substantially complementary to the target INHBE sequence and comprise a contiguous nucleotide sequence which is at least 80% complementary over its entire length to the equivalent region of the nucleotide sequence of any one of SEQ ID NOs:1, 3, 5, 7, or 9, or a fragment of any one of SEQ ID NOs:1, 3, 5, 7, or 9, such as about 85%, about 90%, about 91%, about 92%, about 93%, about 94%, about 95%, about 96%, about 97%, about 98%, or about 99% complementary.

In other embodiments, the antisense polynucleotides disclosed herein are substantially complementary to the target INHBE sequence and comprise a contiguous nucleotide sequence which is at least about 80% complementary over its entire length to any one of the sense strand nucleotide sequences in any one of any one of Tables 2-5, or a fragment of any one of the sense strand nucleotide sequences in any one of Tables 2-5, such as about 85%, about 90%, about 91%, about 92%, about 93%, about 94%, about 95%, about 96%, about 97%, about 98%, about 99%, or 100% complementary.

In one embodiment, an RNAi agent of the disclosure includes a sense strand that is substantially complementary to an antisense polynucleotide which, in turn, is the same as a target INHBE sequence, and wherein the sense strand polynucleotide comprises a contiguous nucleotide sequence which is at least about 80% complementary over its entire length to the equivalent region of the nucleotide sequence of SEQ ID NOs: 2, 4, 6, 8, or 10, or a fragment of any one of SEQ ID NOs:2, 4, 6, 8, or 10, such as about 85%, about 90%, about 91%, about 92%, about 93%, about 94%, about 95%, about 96%, about 97%, about 98%, about 99%, or 100% complementary.

In some embodiments, an iRNA of the invention includes a sense strand that is substantially complementary to an antisense polynucleotide which, in turn, is complementary to a target INHBE sequence, and wherein the sense strand polynucleotide comprises a contiguous nucleotide sequence which is at least about 80% complementary over its entire length to any one of the antisense strand nucleotide sequences in any one of any one of Tables 2-3, or a fragment of any one of the antisense strand nucleotide sequences in any one of Tables 2-3, such as about 85%, about 90%, about 91%, about 92%, about 93%, about 94%, about 95%, about 96%, about 97%, about 98%, about 99%, or 100% complementary.

In general, an “iRNA” includes ribonucleotides with chemical modifications. Such modifications may include all types of modifications disclosed herein or known in the art. Any such modifications, as used in a dsRNA molecule, are encompassed by “iRNA” for the purposes of this specification and claims.

In certain embodiments of the instant disclosure, inclusion of a deoxy-nucleotide if present within an RNAi agent can be considered to constitute a modified nucleotide.

The terms “polynucleotide agent,” “antisense polynucleotide agent” “antisense compound”, and “agent” as used interchangeably herein, refer to an agent comprising a single-stranded oligonucleotide that contains RNA as that term is defined herein, and which targets nucleic acid molecules encoding INHBE (e.g., mRNA encoding INHBE as provided in, for example, any one of SEQ ID NOs:1, 3, 5, 7, or 9). The antisense polynucleotide agents specifically bind to the target nucleic acid molecules via hydrogen bonding (e.g., Watson-Crick, Hoogsteen, or reversed Hoogsteen hydrogen bonding) and interfere with the normal function of the targeted nucleic acid (e.g., by an antisense mechanism of action). This interference with or modulation of the function of a target nucleic acid by the polynucleotide agents of the present invention is referred to as “antisense inhibition.”

The functions of the target nucleic acid molecule to be interfered with may include functions such as, for example, translocation of the RNA to the site of protein translation, translation of protein from the RNA, splicing of the RNA to yield one or more mRNA species, and catalytic activity which may be engaged in or facilitated by the RNA.

In some embodiments, antisense inhibition refers to “inhibiting the expression” of target nucleic acid levels or target protein levels in a cell, e.g., a cell within a subject, such as a mammalian subject, in the presence of the antisense polynucleotide agent complementary to a target nucleic acid as compared to target nucleic acid levels or target protein levels in the absence of the antisense polynucleotide agent. For example, the antisense polynucleotide agents of the invention can inhibit translation in a stoichiometric manner by base pairing to the mRNA and physically obstructing the translation machinery, see Dias, N. et al., (2002) Mol Cancer Ther 1:347-355.

The term “antibody” is used herein in its broadest sense and includes certain types of immunoglobulin molecules comprising one or more antigen-binding domains that specifically bind to an antigen or epitope. The term antibody as used herein refers to a molecule comprising at least complementarity-determining region (CDR) 1, CDR2, and CDR3 of a single domain antibody (sdAb), wherein the molecule is capable of binding to an antigen. The term antibody also refers to molecules comprising at least CDR1, CDR2, and CDR3 of a heavy chain and CDR1, CDR2, and CDR3 of a light chain, wherein the molecule is capable of binding to an antigen. The term antibody also includes fragments that are capable of binding an antigen, such as Fv, single-chain Fv (scFv), Fab, Fab′, and (Fab′)2. The term antibody also includes chimeric antibodies, humanized antibodies, and antibodies of various species such as mouse, human, cynomolgus monkey, llama, camel, etc. The term also includes multivalent antibodies such as bivalent or tetravalent antibodies. A multivalent antibody includes, e.g., a single polypeptide chain comprising multiple antigen binding (CDR-containing) domains, as well as two or more polypeptide chains, each containing one or more antigen binding domains, such two or more polypeptide chains being associated with one another, e.g., through a hinge region capable of forming disulfide bond(s) or any other covalent or noncovalent interaction.

The term “heavy chain variable region” as used herein refers to a region comprising heavy chain CDR1, framework (FR) 2, CDR2, FR3, and CDR3. In some embodiments, a heavy chain variable region also comprises at least a portion of an FR1 and/or at least a portion of an FR4. In some embodiments, a heavy chain CDR1 corresponds to Kabat residues 26 to 35; a heavy chain CDR2 corresponds to Kabat residues 50 to 65; and a heavy chain CDR3 corresponds to Kabat residues 95 to 102. See, e.g., Kabat Sequences of Proteins of Immunological Interest (1987 and 1991, NIH, Bethesda, Md.); and FIG. 1. In some embodiments, a heavy chain CDR1 corresponds to Kabat residues 31 to 35; a heavy chain CDR2 corresponds to Kabat residues 50 to 65; and a heavy chain CDR3 corresponds to Kabat residues 95 to 102. See id.

The term “heavy chain constant region” as used herein refers to a region comprising at least three heavy chain constant domains, CH1, CH2, and CH3. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a constant region is an IgM antibody, and an antibody comprising an a constant region is an IgE antibody. Certain isotypes can be further subdivided into subclasses. For example, IgG antibodies include, but are not limited to, IgG1 (comprising a γ1 constant region), IgG2 (comprising a γ2 constant region), IgG3 (comprising a γ3 constant region), and IgG4 (comprising a γ4 constant region) antibodies; IgA antibodies include, but are not limited to, IgA1 (comprising an al constant region) and IgA2 (comprising an α2 constant region) antibodies; and IgM antibodies include, but are not limited to, IgM1 and IgM2.

The term “heavy chain” (abbreviated HC) as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising light chain CDR1, framework (FR)2, CDR2, FR3, and CDR3. In some embodiments, a light chain variable region also comprises an FR1 and/or an FR4. In some embodiments, a light chain CDR1 corresponds to Kabat residues 24 to 34; a light chain CDR2 corresponds to Kabat residues 50 to 56; and a light chain CDR3 corresponds to Kabat residues 89 to 97. See, e.g., Kabat Sequences of Proteins of Immunological Interest (1987 and 1991, NIH, Bethesda, Md.).

The term “light chain constant region” as used herein refers to a region comprising a light chain constant domain, CL. Nonlimiting exemplary light chain constant regions include λ and κ.

The term “light chain” (abbreviate LC) as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

An “isolated antibody”, as used herein, is intended to refer to an antibody that is substantially free of other antibodies having different antigenic specificities (e.g., an isolated antibody that specifically binds INHBE is substantially free of antibodies that specifically bind antigens other than INHBE). An isolated antibody that specifically binds INHBE may, however, have cross-reactivity to other antigens, such as INHBE molecules from other species. Moreover, an isolated antibody may be substantially free of other cellular material and/or chemicals.

A “chimeric antibody” as used herein refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, a chimeric antibody comprises at least one rat variable region and at least one mouse constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is a sdAb, a Fab, an scFv, a (Fab′)2, etc. The humanized antibody can be selected from any class of immunoglobulins, including IgM, IgG, IgD, IgA and IgE, and any isotype, including without limitation IgG1, IgG2, IgG3 and IgG4. The humanized antibody may comprise sequences from more than one class or isotype, and particular constant domains may be selected to optimize desired effector functions using techniques well-known in the art.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

The terms “an antibody, or antigen binding fragment thereof, that specifically binds INHBE,” or “an anti-INHBE antibody or antigen binding fragment thereof,” used interchangeably herein, refer to an antibody, or antigen binding fragment thereof, that specifically binds to INHBE, e.g., human INHBE. An antibody “which binds” an antigen of interest, i.e., INHBE, is one capable of binding that antigen with sufficient affinity such that the antibody is useful in targeting a cell expressing the antigen. In certain embodiments, the antibody specifically binds to human INHBE. Unless otherwise indicated, the term “anti-INHBE antibody” is meant to refer to an antibody which binds to wild type INHBE, a variant, or an isoform of INHBE.

In one embodiment, the phrase “specifically binds to INHBE” or “specific binding to INHBE”, as used herein, refers to the ability of an anti-INHBE antibody to interact with INHBE with a dissociation constant (K_{D}) of about 2,000 nM or less, about 1,000 nM or less, about 500 nM or less, about 200 nM or less, about 100 nM or less, about 75 nM or less, about 25 nM or less, about 21 nM or less, about 12 nM or less, about 11 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.3 nM or less, about 0.1 nM or less, about 0.01 nM or less, or about 0.001 nM or less. In another embodiment, the phrase “specifically binds to INHBE” or “specific binding to INHBE”, as used herein, refers to the ability of an anti-INHBE antibody to interact with INHBE with a dissociation constant (K_{D}) of between about 1 pM (0.001 nM) to 2,000 nM, between about 500 pM (0.5 nM) to 1,000 nM, between about 500 pM (0.5 nM) to 500 nM, between about 1 nM) to 200 nM, between about 1 nM to 100 nM, between about 1 nM to 50 nM, between about 1 nM to 20 nM, or between about 1 nM to 5 nM. In one embodiment, K_{D} is determined by surface plasmon resonance or by any other method known in the art.

The terms “Kabat numbering,” “Kabat definitions,” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the term “CDR” refers to the complementarity determining region within antibody variable sequences. There are three CDRs in each of the variable regions of the heavy chain (HC) and the light chain (LC), which are designated CDR1, CDR2 and CDR3 (or specifically HC CDR1, HC CDR2, HC CDR3, LC CDR1, LC CDR2, and LC CDR3), for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

The term “epitope” refers to a region of an antigen that is bound by an antibody, or an antibody fragment. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In certain embodiments, an antibody is said to specifically bind an antigen when it preferentially recognizes its target antigen in a complex mixture of proteins and/or macromolecules.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, NJ). For further descriptions, see Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “kₒₙ” or “kₐ”, as used herein, is intended to refer to the on rate constant for association of an antibody to the antigen to form the antibody/antigen complex.

The term “k_{off}” or “k_{d}”, as used herein, is intended to refer to the off rate constant for dissociation of an antibody from the antibody/antigen complex.

The term “K_{D}”, as used herein, is intended to refer to the equilibrium dissociation constant of a particular antibody-antigen interaction. K_{D} is calculated by kₐ/k_{d}. In one embodiment, the antibodies of the invention have a K_{D} of about 2,000 nM or less, about 1,000 nM or less, about 500 nM or less, about 200 nM or less, about 100 nM or less, about 75 nM or less, about 25 nM or less, about 21 nM or less, about 12 nM or less, about 11 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.3 nM or less, about 0.1 nM or less, about 0.01 nM or less, or about 0.001 nM or less.

The phrase “contacting a cell with a modulator,” such as an antisense polynucleotide agent, as used herein, includes contacting a cell by any possible means. Contacting a cell with a moulator includes contacting a cell in vitro with the modulator or contacting a cell in vivo with the modulator. The contacting may be done directly or indirectly. Thus, for example, the modulator may be put into physical contact with the cell by the individual performing the method, or alternatively, the modulator may be put into a situation that will permit or cause it to subsequently come into contact with the cell.

Contacting a cell in vitro may be done, for example, by incubating the cell with the modulator. Contacting a cell in vivo may be done, for example, by injecting the modulator into or near the tissue where the cell is located, or by injecting the modulator into another area, e.g., the bloodstream or the subcutaneous space, such that the modulator will subsequently reach the tissue where the cell to be contacted is located. For example, the modulator, e.g., iRNA, may contain or be coupled to a ligand, e.g., GalNAc, that directs the iRNA to a site of interest, e.g., the liver. Combinations of in vitro and in vivo methods of contacting are also possible. For example, a cell may also be contacted in vitro with a modulator and subsequently transplanted into a subject.

In certain embodiments, contacting a cell with a modulator includes “introducing” or “delivering the modulator into the cell” by facilitating or effecting uptake or absorption into the cell. Absorption or uptake of an iRNA can occur through unaided diffusion or active cellular processes, or by auxiliary agents or devices. Introducing a modulator into a cell may be in vitro or in vivo. For example, for in vivo introduction, a modulator can be injected into a tissue site or administered systemically. In vitro introduction into a cell includes methods known in the art such as electroporation and lipofection. Further approaches are described herein below or are known in the art.

The term “lipid nanoparticle” or “LNP” is a vesicle comprising a lipid layer encapsulating a pharmaceutically active molecule, such as a nucleic acid molecule, e.g., an iRNA or a plasmid from which an iRNA is transcribed. LNPs are described in, for example, U.S. Pat. Nos. 6,858,225, 6,815,432, 8,158,601, and 8,058,069, the entire contents of which are hereby incorporated herein by reference.

As used herein, a “subject” is an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird that expresses the target gene, either endogenously or heterologously. In an embodiment, the subject is a human, such as a human being treated or assessed for a disease or disorder that would benefit from reduction in INHBE expression and/or activity; a human at risk for a disease or disorder that would benefit from reduction in INHBE expression and/or activity; a human having a disease or disorder that would benefit from reduction in INHBE expression and/or activity; or human being treated for a disease or disorder that would benefit from reduction in INHBE expression and/or activity as described herein. In some embodiments, the subject is a female human. In other embodiments, the subject is a male human. In one embodiment, the subject is an adult subject. In another embodiment, the subject is a pediatric subject.

As used herein, the terms “treating” or “treatment” refer to a beneficial or desired result, such as reducing at least one sign or symptom of an INHBE-associated disorder in a subject. Treatment also includes a reduction of one or more sign or symptoms associated with unwanted INHBE expression and/or activity; diminishing the extent of unwanted INHBE activation or stabilization; amelioration or palliation of unwanted INHBE activation or stabilization. “Treatment” can also mean prolonging survival as compared to expected survival in the absence of treatment.

The term “lower” in the context of the level of INHBE in a subject or a disease marker or symptom refers to a statistically significant decrease in such level. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., protein or gene expression level. “Lower” in the context of the level of INHBE in a subject is a decrease to a level accepted as within the range of normal for an individual without such disorder. In certain embodiments, “lower” is the decrease in the difference between the level of a marker or symptom for a subject suffering from a disease and a level accepted within the range of normal for an individual. The term “lower” can also be used in association with normalizing a symptom of a disease or condition, i.e. decreasing the difference between a level in a subject suffering from an INHBE-associated disorder towards or to a level in a normal subject not suffering from an INHBE-associated disorder. As used herein, if a disease is associated with an elevated value for a symptom, “normal” is considered to be the upper limit of normal. If a disease is associated with a decreased value for a symptom, “normal” is considered to be the lower limit of normal.

As used herein, “prevention” or “preventing,” when used in reference to a disease, disorder or condition thereof, may be treated or ameliorated by a reduction in expression and/or activity of INHBE, refers to a reduction in the likelihood that a subject will develop a symptom associated with such a disease, disorder, or condition, e.g., a symptom of an INHBE-associated disorder, e.g., metabolic disorder, e.g., diabetes. The failure to develop a disease, disorder or condition, or the reduction in the development of a symptom associated with such a disease, disorder or condition (e.g., by at least about 10% on a clinically accepted scale for that disease or disorder), or the exhibition of delayed symptoms delayed (e.g., by days, weeks, months or years) is considered effective prevention.

As used herein, the term “inhibin subunit beta E-associated disorder” or “INHBE-associated disorder,” is a disease or disorder that is caused by, or associated with, INHBE gene expression or INHBE protein production and/or activity. The term “INHBE-associated disorder” includes a disease, disorder or condition that would benefit from a decrease in INHBE gene expression, replication, or protein activity. In some embodiments, the INHBE-associated disorder is a metabolic disorder, e.g., metabolic syndrome.

As used herein, a “metabolic disorder” refers to any disease or disorder that disrupts normal metabolism, the process of converting food to energy on a cellular level. Metabolic diseases affect the ability of the cell to perform critical biochemical reactions that involve the processing or transport of proteins (amino acids), carbohydrates (sugars and starches), or lipids (fatty acids). Non-limiting examples of metabolic diseases include disorders of carbohydrates, e.g., diabetes, type I diabetes, type II diabetes, galactosemia, hereditary fructose intolerance, fructose 1,6-diphosphatase deficiency, glycogen storage disorders, congenital disorders of glycosylation, insulin resistance, insulin insufficiency, hyperinsulinemia, impaired glucose tolerance (IGT), abnormal glycogen metabolism; disorders of amino acid metabolism, e.g., maple syrup urine disease (MSUD), or homocystinuria; disorder of organic acid metabolism, e.g., methylmalonic aciduria, 3-methylglutaconic aciduria-Barth syndrome, glutaric aciduria or 2-hydroxyglutaric aciduria—D and L forms; disorders of fatty acid beta-oxidation, e.g., medium-chain acyl-CoA dehydrogenase deficiency (MCAD), long-chain 3-hydroxyacyl-CoA dehydrogenase deficiency (LCHAD), very-long-chain acyl-CoA dehydrogenase deficiency (VLCAD); disorders of lipid metabolism, e.g., GM1 Gangliosidosis, Tay-Sachs Disease, Sandhoff Disease, Fabry Disease, Gaucher Disease, Niemann-Pick Disease, Krabbe Disease, Mucolipidoses, or Mucopolysaccharidoses; mitochondrial disorders, e.g., mitochondrial cardiomyopathies; Leigh disease; mitochondrial encephalopathy, lactic acidosis, and stroke-like episodes (MELAS); myoclonic epilepsy with ragged-red fibers (MERRF); neuropathy, ataxia, and retinitis pigmentosa (NARP); Barth syndrome; peroxisomal disorders, e.g., Zellweger Syndrome (cerebrohepatorenal syndrome), X-Linked Adrenoleukodystrophy or Refsum Disease.

In one embodiment, a metabolic disorder is metabolic syndrome. The term “metabolic syndrome, as used herein, is disorder that includes a clustering of components that reflect overnutrition, sedentary lifestyles, genetic factors, increasing age, and resultant excess adiposity. Metabolic syndrome includes the clustering of abdominal obesity, insulin resistance, dyslipidemia, and elevated blood pressure and is associated with other comorbidities including the prothrombotic state, proinflammatory state, nonalcoholic fatty liver disease, and reproductive disorders. The prevalence of the metabolic syndrome has increased to epidemic proportions not only in the United States and the remainder of the urbanized world but also in developing nations. Metabolic syndrome is associated with an approximate doubling of cardiovascular disease risk and a 5-fold increased risk for incident type 2 diabetes mellitus.

Abdominal adiposity (e.g., a large waist circumference (high waist-to-hip ratio)), high blood pressure, insulin resistance and dislipidemia are central to metabolic syndrome and its individual components (e.g., central obesity, fasting blood glucose (FBG)/pre-diabetes/diabetes, hypercholesterolemia, hypertriglyceridemia, and hypertension).

In one embodiment, a metabolic disorder is a disorder of carbohydrates. In one embodiment, the disorder of carbohydrates is diabetes.

As used herein, the term “diabetes” refers to a group of metabolic disorders characterized by high blood sugar (glucose) levels which result from defects in insulin secretion or action, or both. There are two most common types of diabetes, namely type 1 diabetes and type 2 diabetes, which both result from the body's inability to regulate insulin. Insulin is a hormone released by the pancreas in response to increased levels of blood sugar (glucose) in the blood.

The term “type I diabetes,” as used herein, refers to a chronic disease that occurs when the pancreas produces too little insulin to regulate blood sugar levels appropriately. Type I diabetes is also referred to as insulin-dependent diabetes mellitus, IDDM, and juvenile onset diabetes. People with type I diabetes (insulin-dependent diabetes) produce little or no insulin at all. Although about 6 percent of the United States population has some form of diabetes, only about 10 percent of all diabetics have type I disorder. Most people who have type I diabetes developed the disorder before age 30. Type 1 diabetes represents the result of a progressive autoimmune destruction of the pancreatic β-cells with subsequent insulin deficiency. More than 90 percent of the insulin-producing cells (beta cells) of the pancreas are permanently destroyed. The resulting insulin deficiency is severe, and to survive, a person with type I diabetes must regularly inject insulin.

In type II diabetes (also referred to as noninsulin-dependent diabetes mellitus, NDDM), the pancreas continues to manufacture insulin, sometimes even at higher than normal levels. However, the body develops resistance to its effects, resulting in a relative insulin deficiency. Type II diabetes may occur in children and adolescents but usually begins after age 30 and becomes progressively more common with age: about 15 percent of people over age 70 have type II diabetes. Obesity is a risk factor for type II diabetes, and 80 to 90 percent of the people with this disorder are obese.

In some embodiments, diabetes includes pre-diabetes. “Pre-diabetes” refers to one or more early diabetic conditions including impaired glucose utilization, abnormal or impaired fasting glucose levels, impaired glucose tolerance, impaired insulin sensitivity and insulin resistance. Prediabetes is a major risk factor for the development of type 2 diabetes mellitus, cardiovascular disease and mortality. Much focus has been given to developing therapeutic interventions that prevent the development of type 2 diabetes by effectively treating prediabetes.

Diabetes can be diagnosed by the administration of a glucose tolerance test. Clinically, diabetes is often divided into several basic categories. Primary examples of these categories include, autoimmune diabetes mellitus, non-insulin-dependent diabetes mellitus (type 1 NDDM), insulin-dependent diabetes mellitus (type 2 IDDM), non-autoimmune diabetes mellitus, non-insulin-dependent diabetes mellitus (type 2 NIDDM), and maturity-onset diabetes of the young (MODY). A further category, often referred to as secondary, refers to diabetes brought about by some identifiable condition which causes or allows a diabetic syndrome to develop. Examples of secondary categories include, diabetes caused by pancreatic disease, hormonal abnormalities, drug- or chemical-induced diabetes, diabetes caused by insulin receptor abnormalities, diabetes associated with genetic syndromes, and diabetes of other causes. (see e.g., Harrison's (1996) 14th ed., New York, McGraw-Hill).

In one embodiment, a metabolic disorder is a lipid metabolism disorder. As used herein, a “lipid metabolism disorder” or “disorder of lipid metabolism” refers to any disorder associated with or caused by a disturbance in lipid metabolism. This term also includes any disorder, disease or condition that can lead to hyperlipidemia, or condition characterized by abnormal elevation of levels of any or all lipids and/or lipoproteins in the blood. This term refers to an inherited disorder, such as familial hypertriglyceridemia, familial partial lipodystrophy type 1 (FPLD1), or an induced or acquired disorder, such as a disorder induced or acquired as a result of a disease, disorder or condition (e.g., renal failure), a diet, or intake of certain drugs (e.g., as a result of highly active antiretroviral therapy (HAART) used for treating, e.g., AIDS or HIV).

Additional examples of disorders of lipid metabolism include, but are not limited to, atherosclerosis, dyslipidemia, hypertriglyceridemia (including drug-induced hypertriglyceridemia, diuretic-induced hypertriglyceridemia, alcohol-induced hypertriglyceridemia, β-adrenergic blocking agent-induced hypertriglyceridemia, estrogen-induced hypertriglyceridemia, glucocorticoid-induced hypertriglyceridemia, retinoid-induced hypertriglyceridemia, cimetidine-induced hypertriglyceridemia, and familial hypertriglyceridemia), acute pancreatitis associated with hypertriglyceridemia, chylomicron syndrom, familial chylomicronemia, Apo-E deficiency or resistance, LPL deficiency or hypoactivity, hyperlipidemia (including familial combined hyperlipidemia), hypercholesterolemia, gout associated with hypercholesterolemia, xanthomatosis (subcutaneous cholesterol deposits), hyperlipidemia with heterogeneous LPL deficiency, hyperlipidemia with high LDL and heterogeneous LPL deficiency, fatty liver disease, or non-alcoholic stetohepatitis (NASH).

Cardiovascular diseases are also considered “metabolic disorders”, as defined herein. These diseases may include coronary artery disease (also called ischemic heart disease), hypertension, inflammation associated with coronary artery disease, restenosis, peripheral vascular diseases, and stroke.

Disorders related to body weight are also considered “metabolic disorders”, as defined herein. Such disorders may include obesity, hypo-metabolic states, hypothyroidism, uremia, and other conditions associated with weight gain (including rapid weight gain), weight loss, maintenance of weight loss, or risk of weight regain following weight loss.

Blood sugar disorders are further considered “metabolic disorders”, as defined herein. Such disorders may include diabetes, hypertension, and polycystic ovarian syndrome related to insulin resistance. Other exemplary disorders of metabolic disorders may also include renal transplantation, nephrotic syndrome, Cushing's syndrome, acromegaly, systemic lupus erythematosus, dysglobulinemia, lipodystrophy, glycogenosis type I, and Addison's disease.

In one embodiment, an INHBE-associated disorder is primary hypertension. “Primary hypertension” is a result of environmental or genetic causes (e.g., a result of no obvious underlying medical cause).

In one embodiment, an INHBE-associated disorder is secondary hypertension. “Secondary hypertension” has an identifiable underlying disorder which can be of multiple etiologies, including renal, vascular, and endocrine causes, e.g., renal parenchymal disease (e.g., polycystic kidneys, glomerular or interstitial disease), renal vascular disease (e.g., renal artery stenosis, fibromuscular dysplasia), endocrine disorders (e.g., adrenocorticosteroid or mineralocorticoid excess, pheochromocytoma, hyperthyroidism or hypothyroidism, growth hormone excess, hyperparathyroidism), coarctation of the aorta, or oral contraceptive use.

In one embodiment, an INHBE-associated disorder is resistant hypertension. “Resistant hypertension” is blood pressure that remains above goal (e.g., above 130 mm Hg systolic or above 90 diastolic) in spite of concurrent use of three antihypertensive agents of different classes, one of which is a thiazide diuretic. Subjects whose blood pressure is controlled with four or more medications are also considered to have resistant hypertension.

Additional diseases or conditions related to metabolic disorders that would be apparent to the skilled artisan and are within the scope of this disclosure.

“Therapeutically effective amount,” as used herein, is intended to include the amount of a modulator that, when administered to a subject having an INHBE-associated disorder, is sufficient to effect treatment of the disease (e.g., by diminishing, ameliorating, or maintaining the existing disease or one or more symptoms of disease). The “therapeutically effective amount” may vary depending on the modulator, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of a modulator that, when administered to a subject having an INHBE-associated disorder, is sufficient to prevent or ameliorate the disease or one or more symptoms of the disease. Ameliorating the disease includes slowing the course of the disease or reducing the severity of later-developing disease. The “prophylactically effective amount” may vary depending on the modulator, how the modulator is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of a modulator that produces some desired effect at a reasonable benefit/risk ratio applicable to any treatment. The modulator employed in the methods of the present invention may be administered in a sufficient amount to produce a reasonable benefit/risk ratio applicable to such treatment.

The phrase “pharmaceutically acceptable” is employed herein to refer to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Such carriers are known in the art. Pharmaceutically acceptable carriers include carriers for administration by injection.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs, or localized regions. For example, samples may be derived from particular organs, parts of organs, or fluids or cells within those organs. In certain embodiments, samples may be derived from the liver (e.g., whole liver or certain segments of liver or certain types of cells in the liver, such as, e.g., hepatocytes). In some embodiments, a “sample derived from a subject” refers to urine obtained from the subject. A “sample derived from a subject” can refer to blood or blood derived serum or plasma from the subject.

### II. Modulators of the Invention

The present invention provides modulators, i.e., inhibitors, of INHBE and compostions comprising such modulators for use in modulating the expression and/or activity of INHBE. In some embodiments, the modulators and compositions of the invention are for use in treating a subject, e.g., a mammal, such as a human susceptible to developing an INHBE-associated disorder, e.g., metabolic disorder, e.g., metabolic syndrome, a disorder of carbohydrates, e.g., type II diabetes, pre-diabetes, a lipid metabolism disorder, e.g., a hyperlipidemia, hypertension, a cardiovascular disease, a disorders of body weight.

In one aspect, the present invention provides a modulator of inhibin subunit beta E (INHBE). The modulator may be an oligonucleotide that targets INHBE, such as a double stranded ribonucleic acid (dsRNA) or an antisense polynucleotide agent; an antibody, or antigen-binding fragment thereof, that specifically binds INHBE, such as a monoclonal anti-INHBE antibody, or antigen-binding fragment thereof; a small molecule; a guideRNA that effects ADAR editing, such as a guideRNA that includes a stem loop structure that binds the ADAR enzyme; or a guideRNA that effects CRISPR editing.

In one embodiment, the modulator of the invention is an RNAi, e.g., double stranded ribonucleic acid (dsRNA) agent, targeting an INHBE gene.

In one embodiment, the modulator of the invention is an antisense polynucleotide agent targeting an INHBE gene.

In one embodiment, the modulator of the invention is an antibody, or antigen-binding fragment thereof, that specifically binds INHBE, e.g., a human, humanized or chimeric anti-INHBE antibody, or antigen-binding fragment thereof.

In some embodiments, the modulator of INHBE is a small molecule.

In some embodiments, the modulator of INHBE is an aptamer. In some embodiments, the aptamer is an oligonucleotide aptamer. In some embodiments, the aptamer is a peptide aptamer.

In some embodiments, the modulator of INHBE is a guideRNA that effects double-stranded RNA-specific adenosine deaminase (ADAR) editing, such as a guideRNA that includes a stem loop structure that binds the ADAR enzyme.

In some embodiment, the modulator of INHBE is a guideRNA that effects CRIPR editing.

### A. Oligonucleotides of the Invention that Target INHBE

### i. iRNAs of the Invention

In one embodiment, the oligonucleotide modulator of the invention that targets INHBE is an RNAi.

Accordingly, the present invention provides iRNA compositions which effect the RNA-induced silencing complex (RISC)-mediated cleavage of RNA transcripts of a inhibin subunit beta E (INHBE) gene. The gene may be within a cell, e.g., a cell within a subject, such as a human. The use of these iRNAs enables the targeted degradation of mRNAs of the corresponding gene (INHBE) in mammals.

The iRNAs of the invention have been designed to target the human inhibin subunit beta E (INHBE) gene, including portions of the gene that are conserved in the INHBE orthologs of other mammalian species. Without intending to be limited by theory, it is believed that a combination or sub-combination of the foregoing properties and the specific target sites or the specific modifications in these iRNAs confer to the iRNAs of the invention improved efficacy, stability, potency, durability, and safety.

Accordingly, the present invention provides methods for treating and preventing an inhibin subunit beta E (INHBE)-associated disorder, e.g., a metabolic disorder, e.g. metabolic syndrome, a disorder of carbohydrates, e.g., type II diabetes, pre-diabetes, a lipid metabolism disorder, e.g., a hyperlipidemia, hypertension, a cardiovascular disease, a disorders of body weight, using iRNA compositions which effect the RNA-induced silencing complex (RISC)-mediated cleavage of RNA transcripts of an INHBE gene.

The iRNAs of the invention include an RNA strand (the antisense strand) having a region which is up to about 30 nucleotides or less in length, e.g., 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of an INHBE gene.

In certain embodiments, one or both of the strands of the double stranded RNAi agents of the invention is up to 66 nucleotides in length, e.g., 36-66, 26-36, 25-36, 31-60, 22-43, 27-53 nucleotides in length, with a region of at least 19 contiguous nucleotides that is substantially complementary to at least a part of an mRNA transcript of an INHBE gene. In some embodiments, such iRNA agents having longer length antisense strands may, for example, include a second RNA strand (the sense strand) of 20-60 nucleotides in length wherein the sense and antisense strands form a duplex of 18-30 contiguous nucleotides.

The use of iRNAs of the invention enables the targeted degradation of mRNAs of the corresponding gene (INHBE gene) in mammals. Using in vitro assays, the present inventors have demonstrated that iRNAs targeting an INHBE gene can potently mediate RNAi, resulting in significant inhibition of expression of an INHBE gene. Thus, methods and compositions including these iRNAs are useful for treating a subject having an INHBE-associated disorder, e.g., a metabolic disorder, e.g. metabolic syndrome, a disorder of carbohydrates, e.g., type II diabetes, pre-diabetes, a lipid metabolism disorder, e.g., a hyperlipidemia, hypertension, a cardiovascular disease, a disorders of body weight.

Accordingly, the present invention provides methods and combination therapies for treating a subject having a disorder that would benefit from inhibiting or reducing the expression of an INHBE gene, e.g., a inhibin subunit beta E (INHBE)-associated disease, such as metabolic disorder, e.g., metabolic syndrome, a disorder of carbohydrates, e.g., type II diabetes, pre-diabetes, a lipid metabolism disorder, e.g., a hyperlipidemia, hypertension, a cardiovascular disease, a disorders of body weight, using iRNA compositions which effect the RNA-induced silencing complex (RISC)-mediated cleavage of RNA transcripts of an INHBE gene.

The present invention also provides methods for preventing at least one symptom in a subject having a disorder that would benefit from inhibiting or reducing the expression of an INHBE gene, e.g., a metabolic disorder, e.g. metabolic syndrome, a disorder of carbohydrates, e.g., type II diabetes, pre-diabetes, a lipid metabolism disorder, e.g., a hyperlipidemia, hypertension, a cardiovascular disease, a disorder of body weight.

In one aspect, the present invention provides iRNAs which inhibit the expression of an INHBE gene. In certain embodiments, the iRNA includes double stranded ribonucleic acid (dsRNA) molecules for inhibiting the expression of an INHBE gene in a cell, such as a cell within a subject, e.g., a mammal, such as a human susceptible to developing an INHBE-associated disorder, e.g., metabolic disorder, e.g., metabolic syndrome, a disorder of carbohydrates, e.g., type II diabetes, pre-diabetes, a lipid metabolism disorder, e.g., a hyperlipidemia, hypertension, a cardiovascular disease, a disorders of body weight. The dsRNAi agent includes an antisense strand having a region of complementarity which is complementary to at least a part of an mRNA formed in the expression of an INHBE gene. The region of complementarity is about 19-30 nucleotides in length (e.g., about 30, 29, 28, 27, 26, 25, 24, 23, 22, 21, 20, or 19 nucleotides in length).

Upon contact with a cell expressing the INHBE gene, the iRNA inhibits the expression of the INHBE gene (e.g., a human, a primate, a non-primate, or a rat INHBE gene) by at least about 50% as assayed by, for example, a PCR or branched DNA (bDNA)-based method, or by a protein-based method, such as by immunofluorescence analysis, using, for example, western blotting or flow cytometric techniques. In certain embodiments, inhibition of expression is determined by the qPCR method provided in the examples herein with the siRNA at, e.g., a 10 nM concentration, in an appropriate organism cell line provided therein. In certain embodiments, inhibition of expression in vivo is determined by knockdown of the human gene in a rodent expressing the human gene, e.g., a mouse or an AAV-infected mouse expressing the human target gene, e.g., when administered as single dose, e.g., at 3 mg/kg at the nadir of RNA expression.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of an INHBE gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. In certain embodiments, the duplex structure is 18 to 25 base pairs in length, e.g., 18-25, 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 base pairs in length, for example, 19-21 basepairs in length. Ranges and lengths intermediate to the above recited ranges and lengths are also contemplated to be part of the disclosure.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above recited ranges and lengths are also contemplated to be part of the disclosure.

In some embodiments, the duplex structure is 19 to 30 base pairs in length. Similarly, the region of complementarity to the target sequence is 19 to 30 nucleotides in length.

In some embodiments, the dsRNA is about 19 to about 23 nucleotides in length, or about 25 to about 30 nucleotides in length. In general, the dsRNA is long enough to serve as a substrate for the Dicer enzyme. For example, it is well-known in the art that dsRNAs longer than about 21-23 nucleotides in length may serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 19 to about 30 base pairs, e.g., about 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an iRNA agent useful to target INHBE gene expression is not generated in the target cell by cleavage of a larger dsRNA.

A dsRNA as described herein can further include one or more single-stranded nucleotide overhangs, e.g., 1-4, 2-4, 1-3, 2-3, 1, 2, 3, or 4 nucleotides. dsRNAs having at least one nucleotide overhang can have superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of an antisense or sense strand of a dsRNA.

A dsRNA can be synthesized by standard methods known in the art. Double stranded RNAi compounds of the invention may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Similarly, single-stranded oligonucleotides of the invention can be prepared using solution-phase or solid-phase organic synthesis or both.

In an aspect, a dsRNA of the invention includes at least two nucleotide sequences, a sense sequence and an anti-sense sequence. The sense strand is selected from the group of sequences provided in any one of Tables 2-3, and the corresponding antisense strand of the sense strand is selected from the group of sequences of any one of Tables 2-3. In this aspect, one of the two sequences is complementary to the other of the two sequences, with one of the sequences being substantially complementary to a sequence of an mRNA generated in the expression of an INHBE gene. As such, in this aspect, a dsRNA will include two oligonucleotides, where one oligonucleotide is described as the sense strand in any one of Tables 2-3, and the second oligonucleotide is described as the corresponding antisense strand of the sense strand in any one of Tables 2-3.

In certain embodiments, the substantially complementary sequences of the dsRNA are contained on separate oligonucleotides. In other embodiments, the substantially complementary sequences of the dsRNA are contained on a single oligonucleotide.

It will be understood that, although the sequences in, for example, Table 3, are not described as modified or conjugated sequences, the RNA of the iRNA of the invention e.g., a dsRNA of the invention, may comprise any one of the sequences set forth in any one of Tables 2-3 that is un-modified, un-conjugated, or modified or conjugated differently than described therein. In other words, the invention encompasses dsRNA of Tables 2-3 which are un-modified, un-conjugated, modified, or conjugated, as described herein.

The skilled person is well aware that dsRNAs having a duplex structure of about 20 to 23 base pairs, e.g., 21, base pairs have been hailed as particularly effective in inducing RNA interference (Elbashir et al., EMBO 2001, 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided in any one of Tables 2-3. dsRNAs described herein can include at least one strand of a length of minimally 21 nucleotides. It can be reasonably expected that shorter duplexes having any one of the sequences in any one of Tables 2-3 minus only a few nucleotides on one or both ends can be similarly effective as compared to the dsRNAs described above. Hence, dsRNAs having a sequence of at least 19, 20, or more contiguous nucleotides derived from any one of the sequences of any one of Tables 2-3, and differing in their ability to inhibit the expression of an INHBE gene by not more than about 5, 10, 15, 20, 25, or 30% inhibition from a dsRNA comprising the full sequence, are contemplated to be within the scope of the present invention.

In addition, the RNAs provided in Tables 2-3 identify a site(s) in an INHBE transcript that is susceptible to RISC-mediated cleavage. As such, the present invention further features iRNAs that target within one of these sites. As used herein, an iRNA is said to target within a particular site of an RNA transcript if the iRNA promotes cleavage of the transcript anywhere within that particular site. Such an iRNA will generally include at least about 19 contiguous nucleotides from any one of the sequences provided in any one of Tables 2-3 coupled to additional nucleotide sequences taken from the region contiguous to the selected sequence in an INHBE gene.

### ii. Antisense Polynucleotide Agents of the Invention

In one embodiment, the modulator of the invention is an antisense polynucleotide agent.

Accordingly, the present invention provides polynucleotide agents, e.g., antisense polynucleotide agents, and compositions comprising such agents, which target an INHBE gene and inhibit the expression of the INHBE gene. In one embodiment, the polynucleotide agents, e.g., antisense polynucleotide agents, inhibit the expression of an INHBE gene in a cell, such as a cell within a subject, e.g., a mammal, such as a human having an INHBE-associated disease, e.g., acromegaly, gigantism, or cancer.

The polynucleotide agents of the invention, e.g., antisense polynucleotide agents, include a region of complementarity which is complementary to at least a part of an mRNA formed in the expression of an INHBE gene. The region of complementarity may be about 50 nucleotides or less in length (e.g., 22-12, 20-14, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 nucleotides or less in length). Upon contact with a cell expressing the INHBE gene, the antisense polynucleotide agent inhibits the expression of the INHBE gene (e.g., a human, a primate, a non-primate, or a bird INHBE gene) by at least 20% as assayed by, for example, a PCR or branched DNA (bDNA)-based method, or by a protein-based method, such as by immunofluorescence analysis, using, for example, western blotting, or flow cytometric techniques. In preferred embodiments, the inhibition of expression is determined at a 10 nM concetration using the cell line, delivery method.

The region of complementarity between an antisense polynucleotide agent and a target sequence may be substantially complementary (e.g., there is a sufficient degree of complementarity between the antisense polynucleotide agent and a target nucleic acid to so that they specifically hybridize and induce a desired effect), but is generally fully complementary to the target sequence. The target sequence can be derived from the sequence of an mRNA formed during the expression of an INHBE gene.

Accordingly, in one aspect, an antisense polynucleotide agent of the invention specifically hybridizes to a target nucleic acid molecule, such as the mRNA encoding INHBE, and comprises a contiguous nucleotide sequence which corresponds to the reverse complement of a nucleotide sequence of any one of SEQ ID NOs:1, 3, 5, 7, or 9, or a fragment of any one of SEQ ID NOs:1, 3, 5, 7, or 9.

In some embodiments, the antisense polynucleotide agents of the invention may be substantially complementary to the target sequence. For example, an antisense polynucleotide agent that is substantially complementary to the target sequence may include a contiguous nucleotide sequence comprising no more than 5 mismatches (e.g., no more than 1, no more than 2, no more than 3, no more than 4, or no more than 5 mismatches) when hybridizing to a target sequence, such as to the corresponding region of a nucleic acid which encodes a mammalian INHBE mRNA. In some embodiments, the contiguous nucleotide sequence comprises no more than a single mismatch when hybridizing to the target sequence, such as the corresponding region of a nucleic acid which encodes a mammalian INHBE mRNA.

In some embodiments, the antisense polynucleotide agents of the invention that are substantially complementary to the target sequence comprise a contiguous nucleotide sequence which is at least 80% complementary over its entire length to the equivalent region of the nucleotide sequence of any one of SEQ ID NOs:1, 3, 5, 7, or 9, or a fragment of any one of SEQ ID NOs:1, 3, 5, 7, or 9, such as at least 85%, 90%, 95%, or 100% complementary.

In some embodiments, an antisense polynucleotide agent comprises a contiguous nucleotide sequence which is fully complementary over its entire length to the equivalent region of the nucleotide sequence of any one of SEQ ID NOs: 1, 3, 5, 7, or 9 (or a fragment of any one of SEQ ID NOs:1-5). For example, the nucleotide sequence of an antisense polynucleotide agent is fully complementary over its entire length to the equivalent region of nucleotides 1-20 of GenBank Accession No. NM_031479.5 (SEQ ID NO:1) (see, e.g., Table 4 or 5).

An antisense polynucleotide agent may comprise a contiguous nucleotide sequence of about 4 to 50 nucleotides in length, or any subrange falling within that range, e.g., about 8-49, 8-48, 8-47, 8-46, 8-45, 8-44, 8-43, 8-42, 8-41, 8-40, 8-39, 8-38, 8-37, 8-36, 8-35, 8-34, 8-33, 8-32, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 8-12, 8-11, 8-10, 8-9, 10-49, 10-48, 10-47, 10-46, 10-45, 10-44, 10-43, 10-42, 10-41, 10-40, 10-39, 10-38, 10-37, 10-36, 10-35, 10-34, 10-33, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 10-14, 10-13, 10-12, 10-11, 11-49, 11-48, 11-47, 11-46, 11-45, 11-44, 11-43, 11-42, 11-41, 11-40, 11-39, 11-38, 11-37, 11-36, 11-35, 11-34, 11-33, 11-32, 11-31, 11-30, 11-29, 11-28, 11-27, 11-26, 11-25, 11-24, 11-23, 11-22, 11-21, 11-20, 11-19, 11-18, 11-17, 11-16, 11-15, 11-14, 11-13, 11-12, 12-49, 12-48, 12-47, 12-46, 12-45, 12-44, 12-43, 12-42, 12-41, 12-40, 12-39, 12-38, 12-37, 12-36, 12-35, 12-34, 12-33, 12-32, 12-31, 12-30, 12-29, 12-28, 12-27, 12-26, 12-25, 12-24, 12-23, 12-22, 12-21, 12-20, 12-19, 12-18, 12-17, 12-16, 12-15, 12-14, 12-13, 13-49, 13-48, 13-47, 13-46, 13-45, 13-44, 13-43, 13-42, 13-41, 13-40, 13-39, 13-38, 13-37, 13-36, 13-35, 13-34, 13-33, 13-32, 13-31, 13-30, 13-29, 13-28, 13-27, 13-26, 13-25, 13-24, 13-23, 13-22, 13-21, 13-20, 13-19, 13-18, 13-17, 13-16, 13-15, 13-14, 14-49, 14-48, 14-47, 14-46, 14-45, 14-44, 14-43, 14-42, 14-41, 14-40, 14-39, 14-38, 14-37, 14-36, 14-35, 14-34, 14-33, 14-32, 14-31, 14-30, 14-29, 14-28, 14-27, 14-26, 14-25, 14-24, 14-23, 14-22, 14-21, 14-20, 14-19, 14-18, 14-17, 14-16, 14-15, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 15-16, 16-49, 16-48, 16-47, 16-46, 16-45, 16-44, 16-43, 16-42, 16-41, 16-40, 16-39, 16-38, 16-37, 16-36, 16-35, 16-34, 16-33, 16-32, 16-31, 16-30, 16-29, 16-28, 16-27, 16-26, 16-25, 16-24, 16-23, 16-22, 16-21, 16-20, 16-19, 16-18, 16-17, 17-49, 17-48, 17-47, 17-46, 17-45, 17-44, 17-43, 17-42, 17-41, 17-40, 17-39, 17-38, 17-37, 17-36, 17-35, 17-34, 17-33, 17-32, 17-31, 17-30, 17-29, 17-28, 17-27, 17-26, 17-25, 17-24, 17-23, 17-22, 17-21, 17-20, 17-19, 17-18, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-30, 18-29, 18-28, 18-27, 18-26, 18-25, 18-24, 18-23, 18-22, 18-21, 18-20, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-30, 19-29, 19-28, 19-27, 19-26, 19-25, 19-24, 19-23, 19-22, 19-21, 19-20, 20-49, 20-48, 20-47, 20-46, 20-45, 20-44, 20-43, 20-42, 20-41, 20-40, 20-39, 20-38, 20-37, 20-36, 20-35, 20-34, 20-33, 20-32, 20-31, 20-30, 20-29, 20-28, 20-27, 20-26, 20-25, 20-24, 20-23, 20-22, 20-21, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-30, 21-29, 21-28, 21-27, 21-26, 21-25, 21-24, 21-23, 21-22, 22-49, 22-48, 22-47, 22-46, 22-45, 22-44, 22-43, 22-42, 22-41, 22-40, 22-39, 22-38, 22-37, 22-36, 22-35, 22-34, 22-33, 22-32, 22-31, 22-30, 22-29, 22-28, 22-27, 22-26, 22-25, 22-24, 22-23, 23-49, 23-48, 23-47, 23-46, 23-45, 23-44, 23-43, 23-42, 23-41, 23-40, 23-39, 23-38, 23-37, 23-36, 23-35, 23-34, 23-33, 23-32, 23-31, 23-30, 23-29, 23-28, 23-27, 23-26, 23-25, 23-24, 24-49, 24-48, 24-47, 24-46, 24-45, 24-44, 24-43, 24-42, 24-41, 24-40, 24-39, 24-38, 24-37, 24-36, 24-35, 24-34, 24-33, 24-32, 24-31, 24-30, 24-29, 24-28, 24-27, 24-26, 24-25, 25-49, 25-48, 25-47, 25-46, 25-45, 25-44, 25-43, 25-42, 25-41, 25-40, 25-39, 25-38, 25-37, 25-36, 25-35, 25-34, 25-33, 25-32, 25-31, 25-30, 25-29, 25-28, 25-27, 25-26, 26-49, 26-48, 26-47, 26-46, 26-45, 26-44, 26-43, 26-42, 26-41, 26-40, 26-39, 26-38, 26-37, 26-36, 26-35, 26-34, 26-33, 26-32, 26-31, 26-30, 26-29, 26-28, 26-27, 27-49, 27-48, 27-47, 27-46, 27-45, 27-44, 27-43, 27-42, 27-41, 27-40, 27-39, 27-38, 27-37, 27-36, 27-35, 27-34, 27-33, 27-32, 27-31, 27-30, 27-29, 27-28, 28-49, 28-48, 28-47, 28-46, 28-45, 28-44, 28-43, 28-42, 28-41, 28-40, 28-39, 28-38, 28-37, 28-36, 28-35, 28-34, 28-33, 28-32, 28-31, 28-30, 28-29, 29-49, 29-48, 29-47, 29-46, 29-45, 29-44, 29-43, 29-42, 29-41, 29-40, 29-39, 29-38, 29-37, 29-36, 29-35, 29-34, 29-33, 29-32, 29-31, 29-30, 30-49, 30-48, 30-47, 30-46, 30-45, 30-44, 30-43, 30-42, 30-41, 30-40, 30-39, 30-38, 30-37, 30-36, 30-35, 30-34, 30-33, 30-32, or 30-31 nucleotides in length, e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, an antisense polynucleotide agent may comprise a contiguous nucleotide sequence of no more than 22 nucleotides, e.g., no more than any of 21 nucleotides, 20 nucleotides, 19 nucleotides, no more than 18 nucleotides, 17 nucleotides, 16 nucleotides, than 15 nucleotides, or 14 nucleotides. In other embodiments, the antisense polynucleotide agents of the invention are 20 nucleotides in length. In other embodiments, the antisense polynucleotide agents of the invention are 14 nucleotides in length. In certain embodiments, the polynucleotide is at least 12 nucleotides in length.

In one aspect, an antisense polynucleotide agent of the invention includes a sequence selected from sequences provided in Table 4 or Table 5. It will be understood that, although the sequences in Table 5 are described as modified or conjugated sequences, an antisense polynucleotide agent of the invention, may also comprise any one of the sequences set forth in Table 5 that is un-modified, un-conjugated, or modified or conjugated differently than described therein.

By virtue of the nature of the nucleotide sequences provided in Table 4 or 5, antisense polynucleotide agents of the invention may include one of the sequences of Table 3 or 5 minus only a few nucleotides on one or both ends and yet remain similarly effective as compared to the antisense polynucleotide agents described above. Hence, antisense polynucleotide agents having a sequence of at least 4, 5, 6, 7, 8, 9, 10, 11, 12, 13, 14, 15, 16, 17, 18, 19, or 20 contiguous nucleotides derived from one of the sequences of Table 4 or 5 and differing in their ability to inhibit the expression of an INHBE gene by not more than 5, 10, 15, 20, 25, or 30% inhibition from an antisense polynucleotide agent comprising the full sequence, are contemplated to be within the scope of the present invention. In addition, the antisense polynucleotide agents provided in Table 4 and 5 identify a region(s) in an INHBE transcript that is susceptible to antisense inhibition (e.g., the regions encompassed by the start and end positions relative to the in nucleotide sequences in Table 4). As such, the present invention further features antisense polynucleotide agents that target within one of these sites.

As used herein, an antisense polynucleotide agent is said to target within a particular site of an RNA transcript if the antisense polynucleotide agent promotes antisense inhibition of the target at that site. Such an antisense polynucleotide agent will generally include at least 14 contiguous nucleotides from one of the sequences provided in Table 4 or 5 coupled to additional nucleotide sequences taken from the region contiguous to the selected sequence in an INHBE gene.

While a target sequence is generally 4-50 nucleotides in length, there is wide variation in the suitability of particular sequences in this range for directing antisense inhibition of any given target RNA. Various software packages and the guidelines set out herein provide guidance for the identification of optimal target sequences for any given gene target, but an empirical approach can also be taken in which a “window” or “mask” of a given size (as a non-limiting example, 20 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an antisense polynucleotide agent, mediate the best inhibition of target gene expression. Thus, while the sequences identified, for example, in Table 4 or 5 represent effective target sequences, it is contemplated that further optimization of antisense inhibition efficiency can be achieved by progressively “walking the window” one nucleotide upstream or downstream of the given sequences to identify sequences with equal or better inhibition characteristics.

Further, it is contemplated that for any sequence identified, e.g., in Table 4 or 5, further optimization could be achieved by systematically either adding or removing nucleotides to generate longer or shorter sequences and testing those sequences generated by walking a window of the longer or shorter size up or down the target RNA from that point. Again, coupling this approach to generating new candidate targets with testing for effectiveness of antisense polynucleotide agents based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in length, or other modifications as known in the art or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes) as an expression inhibitor.

### iii. Modified Oligonucleotides of the Invention

In certain embodiments, the oligonucleotides of the invention e.g., dsRNA agents or antisense polynucleotide agents, are un-modified, and do not comprise, e.g., chemical modifications or conjugations known in the art and described herein. In other embodiments, the oligonucleotides, of the invention, e.g., a dsRNA or antisense polynucleotide agent, is chemically modified to enhance stability or other beneficial characteristics. In certain embodiments of the invention, substantially all of the nucleotides of an oligonucleotide, e.g., dsRNA agent or antisense polynucleotide agent of the invention are modified. In other embodiments of the invention, all of the nucleotides of an oligonucleotide, e.g., dsRNA agent or antisense polynucleotide agent, or substantially all of the nucleotides of an oligonucleotide, e.g., dsRNA agent or antisense polynucleotide agent, are modified, i.e., not more than 5, 4, 3, 2, or 1 unmodified nucleotides are present in a strand of the oligonucleotide, e.g., dsRNA agent or antisense polynucleotide agent.

The nucleic acids featured in the invention can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of oligonucleotide compounds useful in the embodiments described herein include, but are not limited to oligonucleotides, e.g., RNAs, containing modified backbones or no natural internucleoside linkages. Oligonucleotides, e.g., RNAs having modified backbones include, among others, those that do not have a phosphorus atom in the backbone. For the purposes of this specification, and as sometimes referenced in the art, modified oligonucleotides, e.g., RNAs, that do not have a phosphorus atom in their internucleoside backbone can also be considered to be oligonucleosides. In some embodiments, a modified oligonucleotide will have a phosphorus atom in its internucleoside backbone.

Modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments of the invention, the oligonucleotides, e.g., dsRNA agents or antisense polynucleotide agents, of the invention are in a free acid form. In other embodiments of the invention, the oligonucleotides, e.g., dsRNA agents or antisense polynucleotide agents, are in a salt form. In one embodiment, the oligonucleotides, e.g., dsRNA agents or antisense polynucleotide agents, of the invention are in a sodium salt form. In certain embodiments, when the oligonucleotides, e.g., dsRNA agents or antisense polynucleotide agents, of the invention are in the sodium salt form, sodium ions are present in the agent as counterions for substantially all of the phosphodiester and/or phosphorothiotate groups present in the agent. Oligonucleotides in which substantially all of the phosphodiester and/or phosphorothioate linkages have a sodium counterion include not more than 5, 4, 3, 2, or 1 phosphodiester and/or phosphorothioate linkages without a sodium counterion. In some embodiments, when the oligonucleotides, e.g., dsRNA agents or antisense polynucleotide agents, of the invention are in the sodium salt form, sodium ions are present in the oligonucleotide as counterions for all of the phosphodiester and/or phosphorothiotate groups present in the agent.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Modified oligonucleotide, e.g., RNA, backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH2 component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Suitable RNA mimetics are contemplated for use in oligonucleotides, e.g., dsRNA agents or antisense polynucleotide agents, provided herein, in which both the sugar and the internucleoside linkage, i.e., the backbone, of the nucleotide units are replaced with novel groups. The base units are maintained for hybridization with an appropriate nucleic acid target compound. One such oligomeric compound in which an RNA mimetic that has been shown to have excellent hybridization properties is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative US patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the oligonucleotides, e.g., iRNAs of the invention are described in, for example, in Nielsen et al., Science, 1991, 254, 1497-1500.

Some embodiments featured in the invention include oligonucleotides with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506. The native phosphodiester backbone can be represented as O—P(O)(OH)—OCH2-.

Modified oligonucleotides can also contain one or more substituted sugar moieties. The oligonucleotides, e.g., dsRNA agents or antisense polynucleotide agents, featured herein can include one of the following at the 2′-position: OH; F; O—, S—, or N-alkyl; O—, S—, or N-alkenyl; O—, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)·ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₃)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. Oligonucleotides, e.g., dsRNA agents or antisense polynucleotide agents, can also have sugar mimetics such as cyclobutyl moieties in place of the pentofuranosyl sugar. Representative US patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An oligonucleotide, e.g., dsRNA agent or antisense polynucleotide agent, can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases such as deoxythimidine (dT), 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In some embodiments, an oligonucleotide, e.g., dsRNA agent or antisense polynucleotide agent, of the disclosure can also be modified to include one or more bicyclic sugar moieties. A “bicyclic sugar” is a furanosyl ring modified by a ring formed by the bridging of two carbons, whether adjacent or non-adjacent. A “bicyclic nucleoside” (“BNA”) is a nucleoside having a sugar moiety comprising a ring formed by bridging two carbons, whether adjacent or non-adjacent, of the sugar ring, thereby forming a bicyclic ring system. In certain embodiments, the bridge connects the 4′-carbon and the 2′-carbon of the sugar ring, optionally, via the 2′-acyclic oxygen atom. Thus, in some embodiments an agent of the invention may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH₂—O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193). Examples of bicyclic nucleosides for use in the polynucleotides of the invention include without limitation nucleosides comprising a bridge between the 4′ and the 2′ ribosyl ring atoms. In certain embodiments, the antisense polynucleotide agents of the invention include one or more bicyclic nucleosides comprising a 4′ to 2′ bridge.

A locked nucleoside can be represented by the structure (omitting stereochemistry),

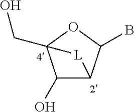

wherein B is a nucleobase or modified nucleobase and L is the linking group that joins the 2′-carbon to the 4′-carbon of the ribose ring. Examples of such 4′ to 2′ bridged bicyclic nucleosides, include but are not limited to 4′-(CH₂)—O-2′ (LNA); 4′-(CH₂)—S-2′; 4′-(CH₂)₂-O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)-2′ (see, e.g., U.S. Patent Publication No. 2004/0171570); 4′-CH₂—N(R)—O-2′, wherein R is H, C1-C12 alkyl, or a nitrogen protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. patents and U.S. patenttent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

A nucleotide of an oligonucleotide, e.g., dsRNA agent or antisense polynucleotide agent, can also be modified to include one or more constrained ethyl nucleotides. As used herein, a “constrained ethyl nucleotide” or “cEt” is a locked nucleic acid comprising a bicyclic sugar moiety comprising a 4′-CH(CH₃)—O-2′ bridge (i.e., L in the preceding structure). In one embodiment, a constrained ethyl nucleotide is in the S conformation referred to herein as “S-cEt.”

An oligonucleotide, e.g., dsRNA agent or antisense polynucleotide agent, of the invention may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, U.S. Patent Publication No. 2013/0190383; and PCT publication WO 2013/036868, the entire contents of each of which are hereby incorporated herein by reference.

In some embodiments, an oligonucleotide, e.g., dsRNA agent or antisense polynucleotide agent, of the invention comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and U.S. Patent Publication Nos. 2013/0096289; 2013/0011922; and 2011/0313020, the entire contents of each of which are hereby incorporated herein by reference.

Potentially stabilizing modifications to the ends of oligonucleotides, e.g., RNA, molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other modifications of the nucleotides of e.g., a dsRNA agent or an antisense polynucleotide agent, of the invention include a 5′ phosphate or 5′ phosphate mimic, e.g., a 5′-terminal phosphate or phosphate mimic on the antisense strand of an iRNA. Suitable phosphate mimics are disclosed in, for example U.S. Patent Publication No. 2012/0157511, the entire contents of which are incorporated herein by reference.

### iv. Modified iRNAs Comprising Motifs of the Invention

In certain aspects of the invention, the double stranded RNA agents of the invention include agents with chemical modifications as disclosed, for example, in WO2013/075035, the entire contents of each of which are incorporated herein by reference. As shown herein and in WO2013/075035, one or more motifs of three identical modifications on three consecutive nucleotides may be introduced into a sense strand or antisense strand of a dsRNAi agent, particularly at or near the cleavage site. In some embodiments, the sense strand and antisense strand of the dsRNAi agent may otherwise be completely modified. The introduction of these motifs interrupts the modification pattern, if present, of the sense or antisense strand. The dsRNAi agent may be optionally conjugated with a GalNAc derivative ligand, for instance on the sense strand.

More specifically, when the sense strand and antisense strand of the double stranded RNA agent are completely modified to have one or more motifs of three identical modifications on three consecutive nucleotides at or near the cleavage site of at least one strand of a dsRNAi agent, the gene silencing activity of the dsRNAi agent was observed.

Accordingly, the invention provides double stranded RNA agents capable of inhibiting the expression of a target gene (i.e., INHBE gene) in vivo. The RNAi agent comprises a sense strand and an antisense strand. Each strand of the RNAi agent may be, for example, 17-30 nucleotides in length, 25-30 nucleotides in length, 27-30 nucleotides in length, 19-25 nucleotides in length, 19-23 nucleotides in length, 19-21 nucleotides in length, 21-25 nucleotides in length, or 21-23 nucleotides in length.

The sense strand and antisense strand typically form a duplex double stranded RNA (“dsRNA”), also referred to herein as “dsRNAi agent.” The duplex region of a dsRNAi agent may be, for example, the duplex region can be 27-30 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 19, 20, 21, 22, 23, 24, 25, 26, and 27 nucleotides in length.

In certain embodiments, the dsRNAi agent may contain one or more overhang regions or capping groups at the 3′-end, 5′-end, or both ends of one or both strands. The overhang can be, independently, 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. In certain embodiments, the overhang regions can include extended overhang regions as provided above. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In certain embodiments, the nucleotides in the overhang region of the dsRNAi agent can each independently be a modified or unmodified nucleotide including, but no limited to 2′-sugar modified, such as, 2′-F, 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof.

For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand, or both strands of the dsRNAi agent may be phosphorylated. In some embodiments, the overhang region(s) contains two nucleotides having a phosphorothioate between the two nucleotides, where the two nucleotides can be the same or different. In some embodiments, the overhang is present at the 3′-end of the sense strand, antisense strand, or both strands. In some embodiments, this 3′-overhang is present in the antisense strand. In some embodiments, this 3′-overhang is present in the sense strand.

The dsRNAi agent may contain only a single overhang, which can strengthen the interference activity of the RNAi, without affecting its overall stability. For example, the single-stranded overhang may be located at the 3′-end of the sense strand or, alternatively, at the 3-end of the antisense strand. The RNAi may also have a blunt end, located at the 5′-end of the antisense strand (i.e., the 3′-end of the sense strand) or vice versa. Generally, the antisense strand of the dsRNAi agent has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In certain embodiments, the dsRNAi agent is a double blunt-ended of 19 nucleotides in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, and 13 from the 5′end.

In other embodiments, the dsRNAi agent is a double blunt-ended of 20 nucleotides in length, wherein the sense strand contains at least one motif of three 2′-F modifications on three consecutive nucleotides at positions 8, 9, and 10 from the 5′end. The antisense strand contains at least one motif of three 2′-O-methyl modifications on three consecutive nucleotides at positions 11, 12, and 13 from the 5′end.

In yet other embodiments, the dsRNAi agent is a double blunt-ended of 21 nucleotides in length, wherein the sense strand contains at least one motif of three 2′-F modifications on three consecutive nucleotides at positions 9, 10, and 11 from the 5′end. The antisense strand contains at least one motif of three 2′-O-methyl modifications on three consecutive nucleotides at positions 11, 12, and 13 from the 5′end.

In certain embodiments, the dsRNAi agent comprises a 21 nucleotide sense strand and a 23 nucleotide antisense strand, wherein the sense strand contains at least one motif of three 2′-F modifications on three consecutive nucleotides at positions 9, 10, and 11 from the 5′end; the antisense strand contains at least one motif of three 2′-O-methyl modifications on three consecutive nucleotides at positions 11, 12, and 13 from the 5′end, wherein one end of the RNAi agent is blunt, while the other end comprises a 2 nucleotide overhang. In one embodiment, the 2 nucleotide overhang is at the 3′-end of the antisense strand.

When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In certain embodiments, every nucleotide in the sense strand and the antisense strand of the dsRNAi agent, including the nucleotides that are part of the motifs are modified nucleotides. In certain embodiments each residue is independently modified with a 2′-O-methyl or 3′-fluoro, e.g., in an alternating motif. Optionally, the dsRNAi agent further comprises a ligand (such as, GalNAc₃).

In certain embodiments, the dsRNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3′terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In certain embodiments, the dsRNAi agent comprises sense and antisense strands, wherein the dsRNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein the 3′ end of the first strand and the 5′ end of the second strand form a blunt end and the second strand is 1-4 nucleotides longer at its 3′ end than the first strand, wherein the duplex region which is at least 25 nucleotides in length, and the second strand is sufficiently complementary to a target mRNA along at least 19 nucleotide of the second strand length to reduce target gene expression when the RNAi agent is introduced into a mammalian cell, and wherein Dicer cleavage of the dsRNAi agent results in an siRNA comprising the 3′-end of the second strand, thereby reducing expression of the target gene in the mammal. Optionally, the dsRNAi agent further comprises a ligand.

In certain embodiments, the sense strand of the dsRNAi agent contains at least one motif of three identical modifications on three consecutive nucleotides, where one of the motifs occurs at the cleavage site in the sense strand.

In certain embodiments, the antisense strand of the dsRNAi agent can also contain at least one motif of three identical modifications on three consecutive nucleotides, where one of the motifs occurs at or near the cleavage site in the antisense strand.

For a dsRNAi agent having a duplex region of 19-23 nucleotides in length, the cleavage site of the antisense strand is typically around the 10, 11, and 12 positions from the 5′-end. Thus the motifs of three identical modifications may occur at the 9, 10, 11 positions; the 10, 11, 12 positions; the 11, 12, 13 positions; the 12, 13, 14 positions; or the 13, 14, 15 positions of the antisense strand, the count starting from the first nucleotide from the 5′-end of the antisense strand, or, the count starting from the first paired nucleotide within the duplex region from the 5′-end of the antisense strand. The cleavage site in the antisense strand may also change according to the length of the duplex region of the dsRNAi agent from the 5′-end.

The sense strand of the dsRNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand.

Alternatively, at least two nucleotides may overlap, or all three nucleotides may overlap.

In some embodiments, the sense strand of the dsRNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adjacent to the first motif or is separated by at least one or more nucleotides. When the motifs are immediately adjacent to each other then the chemistries of the motifs are distinct from each other, and when the motifs are separated by one or more nucleotide than the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the dsRNAi agent may contain more than one motif of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In some embodiments, the wing modification on the sense strand or antisense strand of the dsRNAi agent typically does not include the first one or two terminal nucleotides at the 3′-end, 5′-end, or both ends of the strand.

In other embodiments, the wing modification on the sense strand or antisense strand of the dsRNAi agent typically does not include the first one or two paired nucleotides within the duplex region at the 3′-end, 5′-end, or both ends of the strand.

When the sense strand and the antisense strand of the dsRNAi agent each contain at least one wing modification, the wing modifications may fall on the same end of the duplex region, and have an overlap of one, two, or three nucleotides.

When the sense strand and the antisense strand of the dsRNAi agent each contain at least two wing modifications, the sense strand and the antisense strand can be so aligned that two modifications each from one strand fall on one end of the duplex region, having an overlap of one, two, or three nucleotides; two modifications each from one strand fall on the other end of the duplex region, having an overlap of one, two or three nucleotides; two modifications one strand fall on each side of the lead motif, having an overlap of one, two or three nucleotides in the duplex region.

In some embodiments, every nucleotide in the sense strand and antisense strand of the dsRNAi agent, including the nucleotides that are part of the motifs, may be modified. Each nucleotide may be modified with the same or different modification which can include one or more alteration of one or both of the non-linking phosphate oxygens or of one or more of the linking phosphate oxygens; alteration of a constituent of the ribose sugar, e.g., of the 2′-hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases the modification will occur at all of the subject positions in the nucleic acid but in many cases it will not. By way of example, a modification may only occur at a 3′- or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′-end or ends can be phosphorylated.

It may be possible, e.g., to enhance stability, to include particular bases in overhangs, or to include modified nucleotides or nucleotide surrogates, in single strand overhangs, e.g., in a 5′- or 3′-overhang, or in both. For example, it can be desirable to include purine nucleotides in overhangs. In some embodiments all or some of the bases in a 3′- or 5′-overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, CRN, cET, UNA, HNA, CeNA, 2′-methoxyethyl, 2′-O-methyl, 2′-O-allyl, 2′-C-allyl, 2′-deoxy, 2′-hydroxyl, or 2′-fluoro. The strands can contain more than one modification. In one embodiment, each residue of the sense strand and antisense strand is independently modified with 2′-O-methyl or 2′-fluoro.

At least two different modifications are typically present on the sense strand and antisense strand. Those two modifications may be the 2′-O-methyl or 2′-fluoro modifications, or others.

In certain embodiments, the Nₐ or N_{b} comprise modifications of an alternating pattern. The term “alternating motif” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNAi agent of the invention comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′ to 3′ of the strand and the alternating motif in the antisense strand may start with “BABABA” from 5′ to 3′ of the strand within the duplex region. As another example, the alternating motif in the sense strand may start with “AABBAABB” from 5′ to 3′ of the strand and the alternating motif in the antisense strand may start with “BBAABBAA” from 5′ to 3′ of the strand within the duplex region, so that there is a complete or partial shift of the modification patterns between the sense strand and the antisense strand.

In some embodiments, the dsRNAi agent comprises the pattern of the alternating motif of 2′-O-methyl modification and 2′-F modification on the sense strand initially has a shift relative to the pattern of the alternating motif of 2′-O-methyl modification and 2′-F modification on the antisense strand initially, i.e., the 2′-O-methyl modified nucleotide on the sense strand base pairs with a 2′-F modified nucleotide on the antisense strand and vice versa. The 1 position of the sense strand may start with the 2′-F modification, and the 1 position of the antisense strand may start with the 2′-O-methyl modification.

The introduction of one or more motifs of three identical modifications on three consecutive nucleotides to the sense strand or antisense strand interrupts the initial modification pattern present in the sense strand or antisense strand. This interruption of the modification pattern of the sense or antisense strand by introducing one or more motifs of three identical modifications on three consecutive nucleotides to the sense or antisense strand may enhance the gene silencing activity against the target gene.

In some embodiments, when the motif of three identical modifications on three consecutive nucleotides is introduced to any of the strands, the modification of the nucleotide next to the motif is a different modification than the modification of the motif. For example, the portion of the sequence containing the motif is “ . . . NₐYYYN_{b} . . . ,” where “Y” represents the modification of the motif of three identical modifications on three consecutive nucleotide, and “Nₐ” and “N_{b}” represent a modification to the nucleotide next to the motif “YYY” that is different than the modification of Y, and where Nₐ and N_{b} can be the same or different modifications. Alternatively, Nₐ or N_{b} may be present or absent when there is a wing modification present.

The iRNA may further comprise at least one phosphorothioate or methylphosphonate internucleotide linkage. The phosphorothioate or methylphosphonate internucleotide linkage modification may occur on any nucleotide of the sense strand, antisense strand, or both strands in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand. In one embodiment, a double-stranded RNAi agent comprises 6-8 phosphorothioate internucleotide linkages. In some embodiments, the antisense strand comprises two phosphorothioate internucleotide linkages at the 5′-end and two phosphorothioate internucleotide linkages at the 3′-end, and the sense strand comprises at least two phosphorothioate internucleotide linkages at either the 5′-end or the 3′-end.

In some embodiments, the dsRNAi agent comprises a phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within the duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. These terminal three nucleotides may be at the 3′-end of the antisense strand, the 3′-end of the sense strand, the 5′-end of the antisense strand, or the 5′end of the antisense strand.

In some embodiments, the 2-nucleotide overhang is at the 3′-end of the antisense strand, and there are two phosphorothioate internucleotide linkages between the terminal three nucleotides, wherein two of the three nucleotides are the overhang nucleotides, and the third nucleotide is a paired nucleotide next to the overhang nucleotide. Optionally, the dsRNAi agent may additionally have two phosphorothioate internucleotide linkages between the terminal three nucleotides at both the 5′-end of the sense strand and at the 5′-end of the antisense strand.

In one embodiment, the dsRNAi agent comprises mismatch(es) with the target, within the duplex, or combinations thereof. The mismatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In certain embodiments, the dsRNAi agent comprises at least one of the first 1, 2, 3, 4, or 5 base pairs within the duplex regions from the 5′-end of the antisense strand independently selected from the group of: A:U, G:U, I:C, and mismatched pairs, e.g., non-canonical or other than canonical pairings or pairings which include a universal base, to promote the dissociation of the antisense strand at the 5′-end of the duplex.

In certain embodiments, the nucleotide at the 1 position within the duplex region from the 5′-end in the antisense strand is selected from A, dA, dU, U, and dT. Alternatively, at least one of the first 1, 2, or 3 base pair within the duplex region from the 5′-end of the antisense strand is an AU base pair. For example, the first base pair within the duplex region from the 5′-end of the antisense strand is an AU base pair.

In other embodiments, the nucleotide at the 3′-end of the sense strand is deoxythimidine (dT) or the nucleotide at the 3′-end of the antisense strand is deoxythimidine (dT). For example, there is a short sequence of deoxythimidine nucleotides, for example, two dT nucleotides on the 3′-end of the sense, antisense strand, or both strands.

In certain embodiments, the sense strand sequence may be represented by formula (I):

(I)
5′ nₚ-Nₐ-(X X X )ᵢ-N_{b}-Y Y Y-N_{b}-(Z Z Z)ⱼ-Na-nq 3′

wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 0-10 modified nucleotides;
each nₚ and n_{q} independently represent an overhang nucleotide;
wherein Nb and Y do not have the same modification; and
XXX, YYY, and ZZZ each independently represent one motif of three identical modifications on three consecutive nucleotides. In one embodiment, YYY is all 2′-F modified nucleotides.

In some embodiments, the Nₐ or N_{b} comprises modifications of alternating pattern.

In some embodiments, the YYY motif occurs at or near the cleavage site of the sense strand.

For example, when the dsRNAi agent has a duplex region of 17-23 nucleotides in length, the YYY motif can occur at or the vicinity of the cleavage site (e.g.: can occur at positions 6, 7, 8; 7, 8, 9; 8, 9, 10; 9, 10, 11; 10, 11,12; or 11, 12, 13) of the sense strand, the count starting from the first nucleotide, from the 5′-end; or optionally, the count starting at the first paired nucleotide within the duplex region, from the 5′-end.

In one embodiment, i is 1 and j is 0, or i is 0 and j is 1, or both i and j are 1. The sense strand can therefore be represented by the following formulas:

(Ib)
5′ nₚ-Nₐ-YYY-N_{b}-ZZZ-Nₐ-n_{q} 3′;
(Ic)
5′ nₚ-Nₐ-XXX-N_{b}-YYY-Nₐ-n_{q} 3′;
or
(Id)
5′ nₚ-Nₐ-XXX-N_{b}-YYY-N_{b}-ZZZ-Nₐ-n_{q} 3′.

When the sense strand is represented by formula (Ib), N_{b} represents an oligonucleotide sequence comprising 0-10, 0-7, 0-5, 0-4, 0-2, or 0 modified nucleotides. Each Nₐ independently can represent an oligonucleotide sequence comprising 2-20, 2-15, or 2-10 modified nucleotides.

When the sense strand is represented as formula (Ic), N_{b} represents an oligonucleotide sequence comprising 0-10, 0-7, 0-10, 0-7, 0-5, 0-4, 0-2, or 0 modified nucleotides. Each Nₐ can independently represent an oligonucleotide sequence comprising 2-20, 2-15, or 2-10 modified nucleotides.

When the sense strand is represented as formula (Id), each N_{b} independently represents an oligonucleotide sequence comprising 0-10, 0-7, 0-5, 0-4, 0-2, or 0 modified nucleotides. In one embodiment, N_{b} is 0, 1, 2, 3, 4, 5, or 6 Each Nₐ can independently represent an oligonucleotide sequence comprising 2-20, 2-15, or 2-10 modified nucleotides.

Each of X, Y and Z may be the same or different from each other.

In other embodiments, i is 0 and j is 0, and the sense strand may be represented by the formula:

(Ia)
5′ nₚ-Nₐ-YYY-Nₐ-n_{q} 3′.

When the sense strand is represented by formula (Ia), each Nₐ independently can represent an oligonucleotide sequence comprising 2-20, 2-15, or 2-10 modified nucleotides.

In one embodiment, the antisense strand sequence of the RNAi may be represented by formula (II):

(II)
5′ n_{q′}-Nₐ′-(Z′Z′Z′)ₖ-N_{b}′-Y′Y′Y′-
N_{b}′-(X′X′X′)ₗ-N′ₐ-nₚ′ 3′

wherein:
k and 1 are each independently 0 or 1;
p′ and q′ are each independently 0-6;
each Nₐ′ independently represents an oligonucleotide sequence comprising 0-25 modified nucleotides, each sequence comprising at least two differently modified nucleotides;
each N_{b}′ independently represents an oligonucleotide sequence comprising 0-10 modified nucleotides;
each nₚ′ and n_{q}′ independently represent an overhang nucleotide;
wherein N_{b}‘ and Y’ do not have the same modification; and
X′X′X′, Y′Y′Y′, and Z′Z′Z′ each independently represent one motif of three identical modifications on three consecutive nucleotides.

In some embodiments, the Nₐ′ or N_{b}′ comprises modifications of alternating pattern.

The Y′Y′Y′ motif occurs at or near the cleavage site of the antisense strand. For example, when the dsRNAi agent has a duplex region of 17-23 nucleotides in length, the Y′Y′Y′ motif can occur at positions 9, 10, 11; 10, 11, 12; 11, 12, 13; 12, 13, 14; or 13, 14, 15 of the antisense strand, with the count starting from the first nucleotide, from the 5′-end; or optionally, the count starting at the first paired nucleotide within the duplex region, from the 5′-end. In one embodiment, the Y′Y′Y′ motif occurs at positions 11, 12, 13.

In certain embodiments, Y′Y′Y′ motif is all 2′-OMe modified nucleotides.

In certain embodiments, k is 1 and 1 is 0, or k is 0 and 1 is 1, or both k and 1 are 1.

The antisense strand can therefore be represented b the following formulas:

(IIb)
5′ n_{q′}-Nₐ′-Z′Z′Z′-N_{b}′-Y′Y′Y′-Nₐ′-n_{p′} 3′;
(IIc)
5′ n_{q′}-Nₐ′-Y′Y′Y′-N_{b}′-X′X′X′-n_{p′} 3′;
or
(IId)
5′ n_{q′}-Nₐ′-Z′Z′Z′-ND′-Y′Y′Y′-N_{b}′-
X′X′X′-Nₐ′-n_{p′} 3′.

When the antisense strand is represented by formula (IIb), N_{b}′ represents an oligonucleotide sequence comprising 0-10, 0-7, 0-10, 0-7, 0-5, 0-4, 0-2, or 0 modified nucleotides. Each Nₐ′ independently represents an oligonucleotide sequence comprising 2-20, 2-15, or 2-10 modified nucleotides.

When the antisense strand is represented as formula (IIc), N_{b}′ represents an oligonucleotide sequence comprising 0-10, 0-7, 0-10, 0-7, 0-5, 0-4, 0-2, or 0 modified nucleotides. Each Nₐ′ independently represents an oligonucleotide sequence comprising 2-20, 2-15, or 2-10 modified nucleotides.

When the antisense strand is represented as formula (IId), each N_{b}′ independently represents an oligonucleotide sequence comprising 0-10, 0-7, 0-10, 0-7, 0-5, 0-4, 0-2, or 0 modified nucleotides. Each Nₐ′ independently represents an oligonucleotide sequence comprising 2-20, 2-15, or 2-10 modified nucleotides. In one embodiment, N_{b} is 0, 1, 2, 3, 4, 5, or 6.

In other embodiments, k is 0 and 1 is 0 and the antisense strand may be represented by the formula:

(Ia)
5′ n_{p′}-Nₐ′-Y′Y′Y′-Nₐ′-n_{q′} 3′.

When the antisense strand is represented as formula (IIa), each Nₐ′ independently represents an oligonucleotide sequence comprising 2-20, 2-15, or 2-10 modified nucleotides. Each of X′, Y′ and Z′ may be the same or different from each other.

Each nucleotide of the sense strand and antisense strand may be independently modified with LNA, CRN, UNA, cEt, HNA, CeNA, 2′-methoxyethyl, 2′-O-methyl, 2′-O-allyl, 2′-C-allyl, 2′-hydroxyl, or 2′-fluoro. For example, each nucleotide of the sense strand and antisense strand is independently modified with 2′-O-methyl or 2′-fluoro. Each X, Y, Z, X′, Y′, and Z′, in particular, may represent a 2′-O-methyl modification or a 2′-fluoro modification.

In some embodiments, the sense strand of the dsRNAi agent may contain YYY motif occurring at 9, 10, and 11 positions of the strand when the duplex region is 21 nt, the count starting from the first nucleotide from the 5′-end, or optionally, the count starting at the first paired nucleotide within the duplex region, from the 5′-end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In some embodiments the antisense strand may contain Y′Y′Y′ motif occurring at positions 11, 12, 13 of the strand, the count starting from the first nucleotide from the 5′-end, or optionally, the count starting at the first paired nucleotide within the duplex region, from the 5′-end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with an antisense strand being represented by any one of formulas (IIa), (IIb), (IIc), and (IId), respectively.

Accordingly, the dsRNAi agents for use in the methods of the invention may comprise a sense strand and an antisense strand, each strand having 14 to 30 nucleotides, the iRNA duplex represented b formula (III):

(III)
sense:
5′ nₚ-Nₐ-(X X X)ᵢ-N_{b}-Y Y Y-N_{b}-
(ZZZ)ⱼ-Nₐ-n_{q} 3′
antisense:
3′ nₚ′-Nₐ′-(X′X′X′)ₖ-N_{b}′-Y′Y′Y′-N_{b}′-
(Z′Z′Z′)ₗ-Nₐ′-n_{q}′ 5′

wherein:
i, j, k, and 1 are each independently 0 or 1;
p, p′, q, and q′ are each independently 0-6;
each Nₐ and Nₐ′ independently represents an oligonucleotide sequence comprising 0-25 modified nucleotides, each sequence comprising at least two differently modified nucleotides;
each N_{b} and N_{b}′ independently represents an oligonucleotide sequence comprising 0-10 modified nucleotides;
wherein each nₚ′, nₚ, n_{q}′, and n_{q}, each of which may or may not be present, independently represents an overhang nucleotide; and
XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and 1 is 0; or k is 1 and 1 is 0; k is 0 and 1 is 1; or both k and 1 are 0; or both k and 1 are 1.

Exemplary combinations of the sense strand and antisense strand forming an iRNA duplex include the formulas below:

(IIIa)
5′ nₚ-Nₐ-Y Y Y-Nₐ-n_{q} 3′
3′ nₚ′-Nₐ′-Y′Y′Y′-Nₐ′n_{q}′ 5′
(IIIb)
5′ nₚ-Nₐ-Y Y Y-N_{b}-ZZZ-Nₐ-n_{q} 3′
3′ nₚ′-Nₐ′-Y′Y′Y′-N_{b}′-Z′Z′Z′-Nₐ′n_{q}′ 5′
(IIIc)
5′ nₚ-Nₐ-X X X-N_{b}-Y Y Y-Nₐ-n_{q} 3′
3′ nₚ′-Nₐ′-X′X′X′-N_{b}′-Y′Y′Y′-Nₐ′-n_{q}′ 5′
(IIId)
5′ nₚ-Nₐ-X X X-N_{b}-Y Y Y-N_{b}-ZZZ-Nₐ-n_{q} 3′
3′ nₚ′-Nₐ′-X′X′X′-ND′-Y′Y′Y′-N_{b}′-Z′Z′Z′-Nₐ-n_{q}′ 5′

When the dsRNAi agent is represented by formula (IIIa), each Nₐ independently represents an oligonucleotide sequence comprising 2-20, 2-15, or 2-10 modified nucleotides.

When the dsRNAi agent is represented by formula (IIIb), each N_{b} independently represents an oligonucleotide sequence comprising 1-10, 1-7, 1-5, or 1-4 modified nucleotides. Each Nₐ independently represents an oligonucleotide sequence comprising 2-20, 2-15, or 2-10 modified nucleotides.

When the dsRNAi agent is represented as formula (IIIc), each N_{b}, N_{b}′ independently represents an oligonucleotide sequence comprising 0-10, 0-7, 0-10, 0-7, 0-5, 0-4, 0-2, or 0 modified nucleotides. Each Nₐ independently represents an oligonucleotide sequence comprising 2-20, 2-15, or 2-10 modified nucleotides.

When the dsRNAi agent is represented as formula (IIId), each N_{b}, N_{b}′ independently represents an oligonucleotide sequence comprising 0-10, 0-7, 0-10, 0-7, 0-5, 0-4, 0-2, or 0 modified nucleotides. Each Nₐ, Nₐ′ independently represents an oligonucleotide sequence comprising 2-20, 2-15, or 2-10 modified nucleotides. Each of Nₐ, Nₐ′, N_{b}, and N_{b}′ independently comprises modifications of alternating pattern.

Each of X, Y, and Z in formulas (III), (IIIa), (IIIb), (IIIc), and (IIId) may be the same or different from each other.

When the dsRNAi agent is represented by formula (III), (IIIa), (IIIb), (IIIc), and (IIId), at least one of the Y nucleotides may form a base pair with one of the Y′ nucleotides. Alternatively, at least two of the Y nucleotides form base pairs with the corresponding Y′ nucleotides; or all three of the Y nucleotides all form base pairs with the corresponding Y′ nucleotides.

When the dsRNAi agent is represented by formula (IIIb) or (IIId), at least one of the Z nucleotides may form a base pair with one of the Z′ nucleotides. Alternatively, at least two of the Z nucleotides form base pairs with the corresponding Z′ nucleotides; or all three of the Z nucleotides all form base pairs with the corresponding Z′ nucleotides.

When the dsRNAi agent is represented as formula (IIIc) or (IIId), at least one of the X nucleotides may form a base pair with one of the X′ nucleotides. Alternatively, at least two of the X nucleotides form base pairs with the corresponding X′ nucleotides; or all three of the X nucleotides all form base pairs with the corresponding X′ nucleotides.

In certain embodiments, the modification on the Y nucleotide is different than the modification on the Y′ nucleotide, the modification on the Z nucleotide is different than the modification on the Z′ nucleotide, or the modification on the X nucleotide is different than the modification on the X′ nucleotide.

In certain embodiments, when the dsRNAi agent is represented by formula (IIId), the Nₐ modifications are 2′-O-methyl or 2′-fluoro modifications. In other embodiments, when the RNAi agent is represented by formula (IIId), the Nₐ modifications are 2′-O-methyl or 2′-fluoro modifications and nₚ′>0 and at least one nₚ′ is linked to a neighboring nucleotide a via phosphorothioate linkage. In yet other embodiments, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, and the sense strand is conjugated to one or more GalNAc derivatives attached through a bivalent or trivalent branched linker (described below). In other embodiments, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In some embodiments, when the dsRNAi agent is represented by formula (IIIa), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In some embodiments, the dsRNAi agent is a multimer containing at least two duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In some embodiments, the dsRNAi agent is a multimer containing three, four, five, six, or more duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, two dsRNAi agents represented by at least one of formulas (III), (IIIa), (IIIb), (IIIc), and (IIId) are linked to each other at the 5′ end, and one or both of the 3′ ends, and are optionally conjugated to a ligand. Each of the agents can target the same gene or two different genes; or each of the agents can target same gene at two different target sites.

In certain embodiments, an RNAi agent of the invention may contain a low number of nucleotides containing a 2′-fluoro modification, e.g., 10 or fewer nucleotides with 2′-fluoro modification. For example, the RNAi agent may contain 10, 9, 8, 7, 6, 5, 4, 3, 2, 1 or 0 nucleotides with a 2′-fluoro modification. In a specific embodiment, the RNAi agent of the invention contains 10 nucleotides with a 2′-fluoro modification, e.g., 4 nucleotides with a 2′-fluoro modification in the sense strand and 6 nucleotides with a 2′-fluoro modification in the antisense strand. In another specific embodiment, the RNAi agent of the invention contains 6 nucleotides with a 2′-fluoro modification, e.g., 4 nucleotides with a 2′-fluoro modification in the sense strand and 2 nucleotides with a 2′-fluoro modification in the antisense strand.

In other embodiments, an RNAi agent of the invention may contain an ultra low number of nucleotides containing a 2′-fluoro modification, e.g., 2 or fewer nucleotides containing a 2′-fluoro modification. For example, the RNAi agent may contain 2, 1 of 0 nucleotides with a 2′-fluoro modification. In a specific embodiment, the RNAi agent may contain 2 nucleotides with a 2′-fluoro modification, e.g., 0 nucleotides with a 2-fluoro modification in the sense strand and 2 nucleotides with a 2′-fluoro modification in the antisense strand.

Various publications describe multimeric iRNAs that can be used in the methods of the invention. Such publications include WO2007/091269, U.S. Pat. No. 7,858,769, WO2010/141511, WO2007/117686, WO2009/014887, and WO2011/031520 the entire contents of each of which are hereby incorporated herein by reference.

In certain embodiments, the compositions and methods of the disclosure include a vinyl phosphonate (VP) modification of an RNAi agent as described herein. In exemplary embodiments, a 5′ vinyl phosphonate modified nucleotide of the disclosure has the structure:

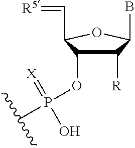

wherein X is O or S;
R is hydrogen, hydroxy, fluoro, or C₁₋₂₀alkoxy (e.g., methoxy or n-hexadecyloxy);
R^{5′} is ═C(H)—P(O)(OH)₂ and the double bond between the C5′ carbon and R^{5′} is in the E or Z orientation (e.g., E orientation); and
B is a nucleobase or a modified nucleobase, optionally where B is adenine, guanine, cytosine, thymine, or uracil.

A vinyl phosphonate of the instant disclosure may be attached to either the antisense or the sense strand of a dsRNA of the disclosure. In certain embodiments, a vinyl phosphonate of the instant disclosure is attached to the antisense strand of a dsRNA, optionally at the 5′ end of the antisense strand of the dsRNA.

Vinyl phosphonate modifications are also contemplated for the compositions and methods of the instant disclosure. An exemplary vinyl phosphonate structure includes the preceding structure, where R5′ is ═C(H)—OP(O)(OH)2 and the double bond between the C5′ carbon and R5′ is in the E or Z orientation (e.g., E orientation).

As described in more detail below, the iRNA that contains conjugations of one or more carbohydrate moieties to an iRNA can optimize one or more properties of the iRNA. In many cases, the carbohydrate moiety will be attached to a modified subunit of the iRNA. For example, the ribose sugar of one or more ribonucleotide subunits of a iRNA can be replaced with another moiety, e.g., a non-carbohydrate (such as, cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” such as, two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide, or polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The iRNA may be conjugated to a ligand via a carrier, wherein the carrier can be cyclic group or acyclic group. In one embodiment, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a serinol backbone or diethanolamine backbone.

### a. Thermally Destabilizing Modifications

In certain embodiments, a dsRNA molecule can be optimized for RNA interference by incorporating thermally destabilizing modifications in the seed region of the antisense strand. As used herein “seed region” means at positions 2-9 of the 5′-end of the referenced strand. For example, thermally destabilizing modifications can be incorporated in the seed region of the antisense strand to reduce or inhibit off-target gene silencing.

The term “thermally destabilizing modification(s)” includes modification(s) that would result with a dsRNA with a lower overall melting temperature (Tₘ) than the Tₘ of the dsRNA without having such modification(s). For example, the thermally destabilizing modification(s) can decrease the Tₘ of the dsRNA by 1-4° C., such as one, two, three or four degrees Celcius. And, the term “thermally destabilizing nucleotide” refers to a nucleotide containing one or more thermally destabilizing modifications.

It has been discovered that dsRNAs with an antisense strand comprising at least one thermally destabilizing modification of the duplex within the first 9 nucleotide positions, counting from the 5′ end, of the antisense strand have reduced off-target gene silencing activity. Accordingly, in some embodiments, the antisense strand comprises at least one (e.g., one, two, three, four, five or more) thermally destabilizing modification of the duplex within the first 9 nucleotide positions of the 5′ region of the antisense strand. In some embodiments, one or more thermally destabilizing modification(s) of the duplex is/are located in positions 2-9, such as, positions 4-8, from the 5′-end of the antisense strand. In some further embodiments, the thermally destabilizing modification(s) of the duplex is/are located at position 6, 7 or 8 from the 5′-end of the antisense strand. In still some further embodiments, the thermally destabilizing modification of the duplex is located at position 7 from the 5′-end of the antisense strand. In some embodiments, the thermally destabilizing modification of the duplex is located at position 2, 3, 4, 5 or 9 from the 5′-end of the antisense strand.

An iRNA agent comprises a sense strand and an antisense strand, each strand having 14 to 40 nucleotides. The RNAi agent may be represented by formula (L):

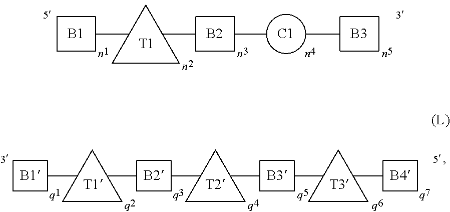

In formula (L), B1, B2, B3, B1′, B2′, B3′, and B4′ each are independently a nucleotide containing a modification selected from the group consisting of 2′-O-alkyl, 2′-substituted alkoxy, 2′-substituted alkyl, 2′-halo, ENA, and BNA/LNA. In one embodiment, B1, B2, B3, B1′, B2′, B3′, and B4′ each contain 2′-OMe modifications. In one embodiment, B1, B2, B3, B1′, B2′, B3′, and B4′ each contain 2′-OMe or 2′-F modifications. In one embodiment, at least one of B1, B2, B3, B1′, B2′, B3′, and B4′ contain 2′-O—N-methylacetamido (2′-O-NMA, 2′O—CH2C(O)N(Me)H) modification.

C1 is a thermally destabilizing nucleotide placed at a site opposite to the seed region of the antisense strand (i.e., at positions 2-8 of the 5′-end of the antisense strand). For example, C1 is at a position of the sense strand that pairs with a nucleotide at positions 2-8 of the 5′-end of the antisense strand. In one example, C1 is at position 15 from the 5′-end of the sense strand. C1 nucleotide bears the thermally destabilizing modification which can include abasic modification; mismatch with the opposing nucleotide in the duplex; and sugar modification such as 2′-deoxy modification or acyclic nucleotide e.g., unlocked nucleic acids (UNA) or glycerol nucleic acid (GNA). In one embodiment, C1 has thermally destabilizing modification selected from the group consisting of: i) mismatch with the opposing nucleotide in the antisense strand; ii) abasic modification selected from the group consisting of:

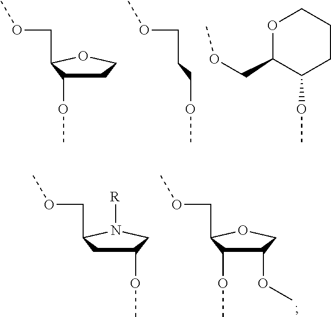

and iii) sugar modification selected from the group consisting of:

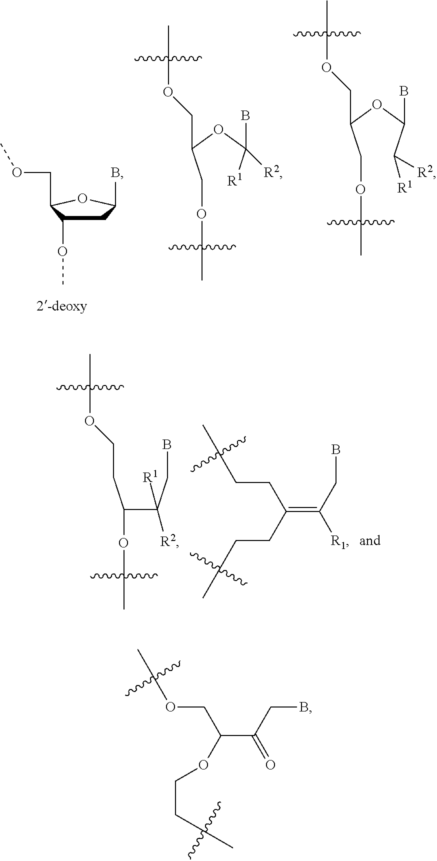

wherein B is a modified or unmodified nucleobase, R¹ and R² independently are H, halogen, OR₃, or alkyl; and R₃ is H, alkyl, cycloalkyl, aryl, aralkyl, heteroaryl or sugar. In one embodiment, the thermally destabilizing modification in C1 is a mismatch selected from the group consisting of G:G, G:A, G:U, G:T, A:A, A:C, C:C, C:U, C:T, U:U, T:T, and U:T; and optionally, at least one nucleobase in the mismatch pair is a 2′-deoxy nucleobase. In one example, the thermally destabilizing modification in C1 is GNA or

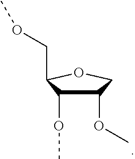

T1, T1′, T2′, and T3′ each independently represent a nucleotide comprising a modification providing the nucleotide a steric bulk that is less or equal to the steric bulk of a 2′-OMe modification. A steric bulk refers to the sum of steric effects of a modification. Methods for determining steric effects of a modification of a nucleotide are known to one skilled in the art. The modification can be at the 2′ position of a ribose sugar of the nucleotide, or a modification to a non-ribose nucleotide, acyclic nucleotide, or the backbone of the nucleotide that is similar or equivalent to the 2′ position of the ribose sugar, and provides the nucleotide a steric bulk that is less than or equal to the steric bulk of a 2′-OMe modification. For example, T1, T1′, T2′, and T3′ are each independently selected from DNA, RNA, LNA, 2′-F, and 2′-F-5′-methyl. In one embodiment, T1 is DNA. In one embodiment, T1′ is DNA, RNA or LNA. In one embodiment, T2′ is DNA or RNA. In one embodiment, T3′ is DNA or RNA.
n¹, n³, and q¹ are independently 4 to 15 nucleotides in length.
n⁵, q³, and q⁷ are independently 1-6 nucleotide(s) in length.
n⁴, q², and q⁶ are independently 1-3 nucleotide(s) in length; alternatively, n⁴ is 0.
q⁵ is independently 0-10 nucleotide(s) in length.
n² and q⁴ are independently 0-3 nucleotide(s) in length.

Alternatively, n⁴ is 0-3 nucleotide(s) in length.

In one embodiment, n⁴ can be 0. In one example, n⁴ is 0, and q² and q⁶ are 1. In another example, n⁴ is 0, and q² and q⁶ are 1, with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴, q², and q⁶ are each 1.

In one embodiment, n², n⁴, q², q⁴, and q⁶ are each 1.

In one embodiment, C1 is at position 14-17 of the 5′-end of the sense strand, when the sense strand is 19-22 nucleotides in length, and n⁴ is 1. In one embodiment, C1 is at position 15 of the 5′-end of the sense strand

In one embodiment, T3′ starts at position 2 from the 5′ end of the antisense strand. In one example, T3′ is at position 2 from the 5′ end of the antisense strand and q⁶ is equal to 1.

In one embodiment, T1′ starts at position 14 from the 5′ end of the antisense strand. In one example, T1′ is at position 14 from the 5′ end of the antisense strand and q² is equal to 1.

In an exemplary embodiment, T3′ starts from position 2 from the 5′ end of the antisense strand and T1′ starts from position 14 from the 5′ end of the antisense strand. In one example, T3′ starts from position 2 from the 5′ end of the antisense strand and q⁶ is equal to 1 and T1′ starts from position 14 from the 5′ end of the antisense strand and q² is equal to 1.

In one embodiment, T1′ and T3′ are separated by 11 nucleotides in length (i.e. not counting the T1′ and T3′ nucleotides).

In one embodiment, T1′ is at position 14 from the 5′ end of the antisense strand. In one example, T1′ is at position 14 from the 5′ end of the antisense strand and q² is equal to 1, and the modification at the 2′ position or positions in a non-ribose, acyclic or backbone that provide less steric bulk than a 2′-OMe ribose.

In one embodiment, T3′ is at position 2 from the 5′ end of the antisense strand. In one example, T3′ is at position 2 from the 5′ end of the antisense strand and q⁶ is equal to 1, and the modification at the 2′ position or positions in a non-ribose, acyclic or backbone that provide less than or equal to steric bulk than a 2′-OMe ribose.

In one embodiment, T1 is at the cleavage site of the sense strand. In one example, T1 is at position 11 from the 5′ end of the sense strand, when the sense strand is 19-22 nucleotides in length, and n² is 1. In an exemplary embodiment, T1 is at the cleavage site of the sense strand at position 11 from the 5′ end of the sense strand, when the sense strand is 19-22 nucleotides in length, and n² is 1, In one embodiment, T2′ starts at position 6 from the 5′ end of the antisense strand. In one example, T2′ is at positions 6-10 from the 5′ end of the antisense strand, and q⁴ is 1.

In an exemplary embodiment, T1 is at the cleavage site of the sense strand, for instance, at position 11 from the 5′ end of the sense strand, when the sense strand is 19-22 nucleotides in length, and n² is 1; T1′ is at position 14 from the 5′ end of the antisense strand, and q² is equal to 1, and the modification to T1′ is at the 2′ position of a ribose sugar or at positions in a non-ribose, acyclic or backbone that provide less steric bulk than a 2′-OMe ribose; T2′ is at positions 6-10 from the 5′ end of the antisense strand, and q⁴ is 1; and T3′ is at position 2 from the 5′ end of the antisense strand, and q⁶ is equal to 1, and the modification to T3′ is at the 2′ position or at positions in a non-ribose, acyclic or backbone that provide less than or equal to steric bulk than a 2′-OMe ribose.

In one embodiment, T2′ starts at position 8 from the 5′ end of the antisense strand. In one example, T2′ starts at position 8 from the 5′ end of the antisense strand, and q⁴ is 2.

In one embodiment, T2′ starts at position 9 from the 5′ end of the antisense strand. In one example, T2′ is at position 9 from the 5′ end of the antisense strand, and q⁴ is 1.

In one embodiment, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 6, T1 is 2′F, n² is 3, B2 is 2′-OMe, n³ is 7, n⁴ is 0, B3 is 2′OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 6, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n² is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within positions 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

The RNAi agent can comprise a phosphorus-containing group at the 5′-end of the sense strand or antisense strand. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos), or 5′-deoxy-5′-C-malonyl

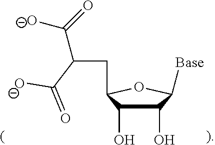

When the 5′-end phosphorus-containing group is 5′-end vinylphosphonate (5′-VP), the 5′-VP can be either 5′-E-VP isomer (i.e., trans-vinylphosphonate,

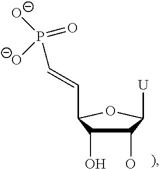

5′-Z—VP isomer (i.e., cis-vinylphosphonate,

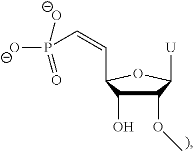

or mixtures thereof.

In one embodiment, the RNAi agent comprises a phosphorus-containing group at the 5′-end of the sense strand. In one embodiment, the RNAi agent comprises a phosphorus-containing group at the 5′-end of the antisense strand.

In one embodiment, the RNAi agent comprises a 5′-P. In one embodiment, the RNAi agent comprises a 5′-P in the antisense strand.

In one embodiment, the RNAi agent comprises a 5′-PS. In one embodiment, the RNAi agent comprises a 5′-PS in the antisense strand.

In one embodiment, the RNAi agent comprises a 5′-VP. In one embodiment, the RNAi agent comprises a 5′-VP in the antisense strand. In one embodiment, the RNAi agent comprises a 5′-E-VP in the antisense strand. In one embodiment, the RNAi agent comprises a 5′-Z—VP in the antisense strand.

In one embodiment, the RNAi agent comprises a 5′-PS₂. In one embodiment, the RNAi agent comprises a 5′-PS₂ in the antisense strand.

In one embodiment, the RNAi agent comprises a 5′-PS₂. In one embodiment, the RNAi agent comprises a 5′-deoxy-5′-C-malonyl in the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-VP. The 5′-VP may be 5′-E-VP, 5′-Z—VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z—VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-VP. The 5′-VP may be 5′-E-VP, 5′-Z—VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-VP. The 5′-VP may be 5′-E-VP, 5′-Z—VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-VP. The 5′-VP may be 5′-E-VP, 5′-Z—VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNAi RNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z—VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-VP. The 5′-VP may be 5′-E-VP, 5′-Z—VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z—VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z—VP, or combination thereof), and a targeting ligand.

In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-VP (e.g., a 5′-E-VP, 5′-Z—VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z—VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z—VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In a particular embodiment, an RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker; and
   (iii) 2′-F modifications at positions 1, 3, 5, 7, 9 to 11, 13, 17, 19, and 21, and 2′-OMe
   modifications at positions 2, 4, 6, 8, 12, 14 to 16, 18, and 20 (counting from the 5′ end); and
(b) an antisense strand having:
   (i) a length of 23 nucleotides;
   (ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
   (iii) phosphorothioate internucleotide linkages between nucleotide positions 21 and 22,
   and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, an RNAi agent of the present invention comprises:
(a) a sense strand having:
(i) a length of 21 nucleotides;
(ii) an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, 13, 15, 17, 19, and 21, and 2′-OMe modifications at positions 2, 4, 6, 8, 12, 14, 16, 18, and 20 (counting from the 5′ end); and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, 10, and 12 to 21, 2′-F modifications at positions 7, and 9, and a deoxy-nucleotide (e.g. dT) at position 11 (counting from the 5′ end); and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, 10, 12, 14, and 16 to 21, and 2′-F modifications at positions 7, 9, 11, 13, and 15; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 5, 7, 9, 11, 13, 15, 17, 19, and 21 to 23, and 2′-F modifications at positions 2 to 4, 6, 8, 10, 12, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 9, and 12 to 21, and 2′-F modifications at positions 10, and 11; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, and 13, and 2′-OMe modifications at positions 2, 4, 6, 8, 12, and 14 to 21; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3, 5 to 7, 9, 11 to 13, 15, 17 to 19, and 21 to 23, and 2′-F modifications at positions 2, 4, 8, 10, 14, 16, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1, 2, 4, 6, 8, 12, 14, 15, 17, and 19 to 21, and 2′-F modifications at positions 3, 5, 7, 9 to 11, 13, 16, and 18; and
   (iv) phosphorothioate internucleotide linkages between nucleotide positions 1 and 2, and between nucleotide positions 2 and 3 (counting from the 5′ end);
and
(b) an antisense strand having:
   (i) a length of 25 nucleotides;
   (ii) 2′-OMe modifications at positions 1, 4, 6, 7, 9, 11 to 13, 15, 17, and 19 to 23, 2′-F modifications at positions 2, 3, 5, 8, 10, 14, 16, and 18, and deoxy-nucleotides (e.g. dT) at positions 24 and 25 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a four nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 19 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 4, 6, and 10 to 19, and 2′-F modifications at positions 5, and 7 to 9; and
   (iv) phosphorothioate internucleotide linkages between nucleotide positions 1 and 2, and between nucleotide positions 2 and 3 (counting from the 5′ end);
and
(b) an antisense strand having:
   (i) a length of 21 nucleotides;
   (ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 19 and 20, and between nucleotide positions 20 and 21 (counting from the 5′ end);
      wherein the RNAi agents have a two nucleotide overhang at the 3′-end of the antisense strand, and a blunt end at the 5′-end of the antisense strand.

In certain embodiments, the iRNA for use in the methods of the invention is an agent selected from agents listed in any one of Tables 4-5. These agents may further comprise a ligand.

### v. Antisense Polynucleotide Agents Comprising Motifs

In certain embodiments of the invention, at least one of the contiguous nucleotides of the antisense polynucleotide agents of the invention may be a modified nucleotide. In one embodiment, the modified nucleotide comprises one or more modified sugars. In other embodiments, the modified nucleotide comprises one or more modified nucleobases. In yet other embodiments, the modified nucleotide comprises one or more modified internucleoside linkages. In some embodiments, the modifications (sugar modifications, nucleobase modifications, or linkage modifications) define a pattern or motif. In one embodiment, the patterns of modifications of sugar moieties, internucleoside linkages, and nucleobases are each independent of one another.

Antisense polynucleotide agents having modified oligonucleotides arranged in patterns, or motifs may, for example, confer to the agents properties such as enhanced inhibitory activity, increased binding affinity for a target nucleic acid, or resistance to degradation by in vivo nucleases. For example, such agents may contain at least one region modified so as to confer increased resistance to nuclease degradation, increased cellular uptake, increased binding affinity for the target nucleic acid, or increased inhibitory activity. A second region of such agents may optionally serve as a substrate for the cellular endonuclease RNase H, which cleaves the RNA strand of an RNA:DNA duplex.

An exemplary antisense polynucleotide agent having modified oligonucleotides arranged in patterns, or motifs is a gapmer. In a “gapmer”, an internal region or “gap” having a plurality of linked nucleotides that supports RNaseH cleavage is positioned between two external flanking regions or “wings” having a plurality of linked nucleotides that are chemically distinct from the linked nucleotides of the internal region. The gap segment generally serves as the substrate for endonuclease cleavage, while the wing segments comprise modified nucleotides.

The three regions of a gapmer motif (the 5′-wing, the gap, and the 3′-wing) form a contiguous sequence of nucleotides and may be described as “X-Y-Z”, wherein “X” represents the length of the 5-wing, “Y” represents the length of the gap, and “Z” represents the length of the 3′-wing. In one embodiment, a gapmer described as “X-Y-Z” has a configuration such that the gap segment is positioned immediately adjacent to each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different.

In certain embodiments, the regions of a gapmer are differentiated by the types of modified nucleotides in the region. The types of modified nucleotides that may be used to differentiate the regions of a gapmer, in some embodiments, include β-D-ribonucleotides, β-D-deoxyribonucleotides, 2′-modified nucleotides, e.g., 2′-modified nucleotides (e.g., 2′-MOE, and 2′-O—CH3), and bicyclic sugar modified nucleotides (e.g., those having a 4′-(CH2)n-O-2′ bridge, where n=1 or n=2).

In one embodiment, at least some of the modified nucleotides of each of the wings may differ from at least some of the modified nucleotides of the gap. For example, at least some of the modified nucleotides of each wing that are closest to the gap (the 3′-most nucleotide of the 5′-wing and the 5′-most nucleotide of the 3-wing) differ from the modified nucleotides of the neighboring gap nucleotides, thus defining the boundary between the wings and the gap. In certain embodiments, the modified nucleotides within the gap are the same as one another. In certain embodiments, the gap includes one or more modified nucleotides that differ from the modified nucleotides of one or more other nucleotides of the gap.

The length of the 5′-wing (X) of a gapmer may be 1 to 6 nucleotides in length, e.g., 2 to 6, 2 to 5, 3 to 6, 3 to 5, 1 to 5, 1 to 4, 1 to 3, 2 to 4 nucleotides in length, e.g., 1, 2, 3, 4, 5, or 6 nucleotides in length.

The length of the 3′-wing (Z) of a gapmer may be 1 to 6 nucleotides in length, e.g., 2 to 6, 2-5, 3 to 6, 3 to 5, 1 to 5, 1 to 4, 1 to 3, 2 to 4 nucleotides in length, e.g., 1, 2, 3, 4, 5, or 6 nucleotides in length.

The length of the gap (Y) of a gapmer may be 5 to 14 nucleotides in length, e.g., 5 to 13, 5 to 12, 5 to 11, 5 to 10, 5 to 9, 5 to 8, 5 to 7, 5 to 6, 6 to 14, 6 to 13, 6 to 12, 6 to 11, 6 to 10, 6 to 9, 6 to 8, 6 to 7, 7 to 14, 7 to 13, 7 to 12, 7 to 11, 7 to 10, 7 to 9, 7 to 8, 8 to 14, 8 to 13, 8 to 12, 8 to 11, 8 to 10, 8 to 9, 9 to 14, 9 to 13, 9 to 12, 9 to 11, 9 to 10, 10 to 14, 10 to 13, 10 to 12, 10 to 11, 11 to 14, 11 to 13, 11 to 12, 12 to 14, 12 to 13, or 13 to 14 nucleotides in length, e.g., 5, 6, 7, 8, 9, 10, 11, 12, 13, or 14 nucleotides in length.

In some embodiments of the invention X consists of 2, 3, 4, 5 or 6 nucleotides, Y consists of 7, 8, 9, 10, 11, or 12 nucleotides, and Z consists of 2, 3, 4, 5 or 6 nucleotides. Such gapmers include (X-Y-Z) 2-7-2, 2-7-3, 2-7-4, 2-7-5, 2-7-6, 3-7-2, 3-7-3, 3-7-4, 3-7-5, 3-7-6, 4-7-3, 4-7-4, 4-7-5, 4-7-6, 5-7-3, 5-7-4, 5-7-5, 5-7-6, 6-7-3, 6-7-4, 6-7-5, 6-7-6, 3-7-3, 3-7-4, 3-7-5, 3-7-6, 4-7-3, 4-7-4, 4-7-5, 4-7-6, 5-7-3, 5-7-4, 5-7-5, 5-7-6, 6-7-3, 6-7-4, 6-7-5, 6-7-6, 2-8-2, 2-8-3, 2-8-4, 2-8-5, 2-8-6, 3-8-2, 3-8-3, 3-8-4, 3-8-5, 3-8-6, 4-8-3, 4-8-4, 4-8-5, 4-8-6, 5-8-3, 5-8-4, 5-8-5, 5-8-6, 6-8-3, 6-8-4, 6-8-5, 6-8-6, 2-9-2, 2-9-3, 2-9-4, 2-9-5, 2-9-6, 3-9-2, 3-9-3, 3-9-4, 3-9-5, 3-9-6, 4-9-3, 4-9-4, 4-9-5, 4-9-6, 5-9-3, 5-9-4, 5-9-5, 5-9-6, 6-9-3, 6-9-4, 6-9-5, 6-9-6, 2-10-2, 2-10-3, 2-10-4, 2-10-5, 2-10-6, 3-10-2, 3-10-3, 3-10-4, 3-10-5, 3-10-6, 4-10-3, 4-10-4, 4-10-5, 4-10-6, 5-10-3, 5-10-4, 5-10-5, 5-10-6, 6-10-3, 6-10-4, 6-10-5, 6-10-6, 2-11-2, 2-11-3, 2-11-4, 2-11-5, 2-11-6, 3-11-2, 3-11-3, 3-11-4, 3-11-5, 3-11-6, 4-11-3, 4-11-4, 4-11-5, 4-11-6, 5-11-3, 5-11-4, 5-11-5, 5-11-6, 6-11-3, 6-11-4, 6-11-5, 6-11-6, 2-12-2, 2-12-3, 2-12-4, 2-12-5, 2-12-6, 3-12-2, 3-12-3, 3-12-4, 3-12-5, 3-12-6, 4-12-3, 4-12-4, 4-12-5, 4-12-6, 5-12-3, 5-12-4, 5-12-5, 5-12-6, 6-12-3, 6-12-4, 6-12-5, or 6-12-6.

In some embodiments of the invention, antisense polynucleotide agents targeting INHBE include a 5-10-5 gapmer motif. In other embodiments of the invention, antisense polynucleotide agents targeting INHBE include a 4-10-4 gapmer motif. In another embodiment of the invention, antisense polynucleotide agents targeting INHBE include a 3-10-3 gapmer motif. In yet other embodiments of the invention, antisense polynucleotide agents targeting INHBE include a 2-10-2 gapmer motif.

The 5′-wing or 3′-wing of a gapmer may independently include 1-6 modified nucleotides, e.g., 1, 2, 3, 4, 5, or 6 modified nucleotides.

In some embodiment, the 5′-wing of a gapmer includes at least one modified nucleotide. In one embodiment, the 5′-wing of a gapmer comprises at least two modified nucleotides. In another embodiment, the 5′-wing of a gapmer comprises at least three modified nucleotides. In yet another embodiment, the 5′-wing of a gapmer comprises at least four modified nucleotides. In another embodiment, the 5′-wing of a gapmer comprises at least five modified nucleotides. In certain embodiments, each nucleotide of the 5′-wing of a gapmer is a modified nucleotide.

In some embodiments, the 3′-wing of a gapmer includes at least one modified nucleotide. In one embodiment, the 3′-wing of a gapmer comprises at least two modified nucleotides. In another embodiment, the 3′-wing of a gapmer comprises at least three modified nucleotides. In yet another embodiment, the 3′-wing of a gapmer comprises at least four modified nucleotides. In another embodiment, the 3′-wing of a gapmer comprises at least five modified nucleotides. In certain embodiments, each nucleotide of the 3′-wing of a gapmer is a modified nucleotide.

In certain embodiments, the regions of a gapmer are differentiated by the types of sugar moieties of the nucleotides. In one embodiment, the nucleotides of each distinct region comprise uniform sugar moieties. In other embodiments, the nucleotides of each distinct region comprise different sugar moieties. In certain embodiments, the sugar nucleotide modification motifs of the two wings are the same as one another. In certain embodiments, the sugar nucleotide modification motifs of the 5′-wing differs from the sugar nucleotide modification motif of the 3′-wing.

The 5′-wing of a gapmer may include 1-6 modified nucleotides, e.g., 1, 2, 3, 4, 5, or 6 modified nucleotides.

In one embodiment, at least one modified nucleotide of the 5′-wing of a gapmer is a bicyclic nucleotide, such as a constrained ethyl nucleotide, or an LNA. In another embodiment, the 5′-wing of a gapmer includes 2, 3, 4, or 5 bicyclic nucleotides. In some embodiments, each nucleotide of the 5′-wing of a gapmer is a bicyclic nucleotide.

In one embodiment, the 5′-wing of a gapmer includes at least 1, 2, 3, 4, or 5 constrained ethyl nucleotides. In some embodiments, each nucleotide of the 5′-wing of a gapmer is a constrained ethyl nucleotide.

In one embodiment, the 5′-wing of a gapmer comprises at least one LNA nucleotide. In another embodiment, the 5′-wing of a gapmer includes 2, 3, 4, or 5 LNA nucleotides. In other embodiments, each nucleotide of the 5′-wing of a gapmer is an LNA nucleotide.

In certain embodiments, at least one modified nucleotide of the 5′-wing of a gapmer is a non-bicyclic modified nucleotide, e.g., a 2′-substituted nucleotide. A “2′-substituted nucleotide” is a nucleotide comprising a modification at the 2′-position which is other than H or OH, such as a 2′-OMe nucleotide, or a 2′-MOE nucleotide. In one embodiment, the 5′-wing of a gapmer comprises 2, 3, 4, or 5 2′-substituted nucleotides. In one embodiment, each nucleotide of the 5′-wing of a gapmer is a 2′-substituted nucleotide.

In one embodiment, the 5′-wing of a gapmer comprises at least one 2′-OMe nucleotide. In one embodiment, the 5′-wing of a gapmer comprises at least 2, 3, 4, or 5 2′-OMe nucleotides. In one embodiment, each of the nucleotides of the 5′-wing of a gapmer comprises a 2′-OMe nucleotide.

In one embodiment, the 5′-wing of a gapmer comprises at least one 2′-MOE nucleotide. In one embodiment, the 5′-wing of a gapmer comprises at least 2, 3, 4, or 5 2′-MOE nucleotides. In one embodiment, each of the nucleotides of the 5′-wing of a gapmer comprises a 2′-MOE nucleotide.

In certain embodiments, the 5′-wing of a gapmer comprises at least one 2′-deoxynucleotide. In certain embodiments, each nucleotide of the 5′-wing of a gapmer is a 2′-deoxynucleotide. In a certain embodiments, the 5′-wing of a gapmer comprises at least one ribonucleotide. In certain embodiments, each nucleotide of the 5′-wing of a gapmer is a ribonucleotide.

The 3′-wing of a gapmer may include 1-6 modified nucleotides, e.g., 1, 2, 3, 4, 5, or 6 modified nucleotides.

In one embodiment, at least one modified nucleotide of the 3′-wing of a gapmer is a bicyclic nucleotide, such as a constrained ethyl nucleotide, or an LNA. In another embodiment, the 3′-wing of a gapmer includes 2, 3, 4, or 5 bicyclic nucleotides. In some embodiments, each nucleotide of the 3′-wing of a gapmer is a bicyclic nucleotide.

In one embodiment, the 3′-wing of a gapmer includes at least one constrained ethyl nucleotide. In another embodiment, the 3′-wing of a gapmer includes 2, 3, 4, or 5 constrained ethyl nucleotides. In some embodiments, each nucleotide of the 3′-wing of a gapmer is a constrained ethyl nucleotide.

In one embodiment, the 3′-wing of a gapmer comprises at least one LNA nucleotide. In another embodiment, the 3′-wing of a gapmer includes 2, 3, 4, or 5 LNA nucleotides. In other embodiments, each nucleotide of the 3′-wing of a gapmer is an LNA nucleotide.

In certain embodiments, at least one modified nucleotide of the 3′-wing of a gapmer is a non-bicyclic modified nucleotide, e.g., a 2′-substituted nucleotide. In one embodiment, the 3′-wing of a gapmer comprises 2, 3, 4, or 5 2′-substituted nucleotides. In one embodiment, each nucleotide of the 3′-wing of a gapmer is a 2′-substituted nucleotide.

In one embodiment, the 3′-wing of a gapmer comprises at least one 2′-OMe nucleotide. In one embodiment, the 3′-wing of a gapmer comprises at least 2, 3, 4, or 5 2′-OMe nucleotides. In one embodiment, each of the nucleotides of the 3′-wing of a gapmer comprises a 2′-OMe nucleotide.

In one embodiment, the 3′-wing of a gapmer comprises at least one 2′-MOE nucleotide. In one embodiment, the 3′-wing of a gapmer comprises at least 2, 3, 4, or 5 2′-MOE nucleotides. In one embodiment, each of the nucleotides of the 3′-wing of a gapmer comprises a 2′-MOE nucleotide.

In certain embodiments, the 3′-wing of a gapmer comprises at least one 2′-deoxynucleotide. In certain embodiments, each nucleotide of the 3′-wing of a gapmer is a 2′-deoxynucleotide. In a certain embodiments, the 3′-wing of a gapmer comprises at least one ribonucleotide. In certain embodiments, each nucleotide of the 3′-wing of a gapmer is a ribonucleotide.

The gap of a gapmer may include 5-14 modified nucleotides, e.g., 5, 6, 7, 8, 9, 10, 11, 12, 13, or 14 modified nucleotides.

In one embodiment, the gap of a gapmer comprises at least one 5-methylcytosine. In one embodiment, the gap of a gapmer comprises at least 2, 3, 4, 5, 6, 7, 8, 9, 10, 11, 12, or 13 5-methylcytosines. In one embodiment, all of the nucleotides of the gap of a gapmer are 5-methylcytosines.

In one embodiment, the gap of a gapmer comprises at least one 2′-deoxynucleotide. In one embodiment, the gap of a gapmer comprises at least 2, 3, 4, 5, 6, 7, 8, 9, 10, 11, 12, or 13 2′-deoxynucleotides. In one embodiment, all of the nucleotides of the gap of a gapmer are 2′-deoxynucleotides.

A gapmer may include one or more modified internucleotide linkages. In some embodiments, a gapmer includes one or more phosphodiester internucleotide linkages. In other embodiments, a gapmer includes one or more phosphorothioate internucleotide linkages.

In one embodiment, each nucleotide of a 5′-wing of a gapmer are linked via a phosphorothioate internucleotide linkage. In another embodiment, each nucleotide of a 3′-wing of a gapmer are linked via a phosphorothioate internucleotide linkage. In yet another embodiment, each nucleotide of a gap segment of a gapmer is linked via a phosphorothioate internucleotide linkage. In one embodiment, all of the nucleotides in a gapmer are linked via phosphorothioate internucleotide linkages.

In one embodiment, an antisense polynucleotide agent targeting an INHBE gene comprises a gap segment of ten 2′-deoxyribonucleotides positioned immediately adjacent to and between a 5′-wing segment comprising five nucleotides and a 3′-wing segment comprising 5 nucleotides.

In another embodiment, an antisense polynucleotide agent targeting an INHBE gene comprises a gap segment of ten 2′-deoxyribonucleotides positioned immediately adjacent to and between a 5′-wing segment comprising four nucleotides and a 3′-wing segment comprising four nucleotides.

In another embodiment, an antisense polynucleotide agent targeting an INHBE gene comprises a gap segment of ten 2′-deoxyribonucleotides positioned immediately adjacent to and between a 5′-wing segment comprising three nucleotides and a 3′-wing segment comprising three nucleotides.

In another embodiment, an antisense polynucleotide agent targeting an INHBE gene comprises a gap segment of ten 2′-deoxyribonucleotides positioned immediately adjacent to and between a 5′-wing segment comprising two nucleotides and a 3′-wing segment comprising two nucleotides.

In one embodiment, each nucleotide of a 5-wing flanking a gap segment of 10 2′-deoxyribonucleotides comprises a modified nucleotide. In another embodiment, each nucleotide of a 3-wing flanking a gap segment of 10 2′-deoxyribonucleotides comprises a modified nucleotide. In one embodiment, each of the modified 5′-wing nucleotides and each of the modified 3′-wing nucleotides comprise a 2′-sugar modification. In one embodiment, the 2′-sugar modification is a 2′-OMe modification. In another embodiment, the 2′-sugar modification is a 2′-MOE modification. In one embodiment, each of the modified 5′-wing nucleotides and each of the modified 3′-wing nucleotides comprise a bicyclic nucleotide. In one embodiment, the bicyclic nucleotide is a constrained ethyl nucleotide. In another embodiment, the bicyclic nucleotide is an LNA nucleotide. In one embodiment, each cytosine in an antisense polynucleotide agent targeting an INHBE gene is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising five nucleotides comprising a 2′OMe modification and a 3′-wing segment comprising five nucleotides comprising a 2′OMe modification, wherein each internucleotide linkage of the agent is a phosphorothioate linkage. In one embodiment, each cytosine of the agent is a 5-methylcytosine. In some embodiments, the agent further comprises a ligand.

In one embodiment, an antisense polynucleotide agent targeting an INHBE gene comprises a gap segment of ten 2′-deoxyribonucleotides positioned immediately adjacent to and between a 5′-wing segment comprising five nucleotides comprising a 2′MOE modification and a 3′-wing segment comprising five nucleotides comprising a 2′MOE modification, wherein each internucleotide linkage of the agent is a phosphorothioate linkage. In one embodiment, each cytosine of the agent is a 5-methylcytosine. In some embodiments, the agent further comprises a ligand.

In one embodiment, an antisense polynucleotide agent targeting an INHBE gene comprises a gap segment of ten 2′-deoxyribonucleotides positioned immediately adjacent to and between a 5′-wing segment comprising five constrained ethyl nucleotides and a 3′-wing segment comprising five constrained ethyl nucleotides, wherein each internucleotide linkage of the agent is a phosphorothioate linkage. In some embodiments, each cytosine of the agent is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising five LNA nucleotides and a 3′-wing segment comprising five LNA nucleotides, wherein each internucleotide linkage of the agent is a phosphorothioate linkage. In some embodiments, each cytosine of the agent is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising four nucleotides comprising a 2′OMe modification and a 3′-wing segment comprising four nucleotides comprising a 2′OMe modification, wherein each internucleotde linkage of the agent is a phosphorothioate linkage. In some embodiments, each cytosine of the agent is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising four nucleotides comprising a 2′MOE modification and a 3′-wing segment comprising four nucleotides comprising a 2′MOE modification, wherein each internucleotide linkage of the agent is a phosphorothioate linkage. In some embodiments, each cytosine of the agent is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising four constrained ethyl nucleotides and a 3′-wing segment comprising four constrained ethyl nucleotides, wherein each internucleoitde linkage of the agent is a phosphorothioate linkage. In some embodiments, each cytosine of the agent is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising four LNA nucleotides and a 3′-wing segment comprising four LNA nucleotides, wherein each internucleotide linkage of the agent is a phosphorothioate linkage. In some embodiments, each cytosine of the agent is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising three nucleotides comprising a 2′OMe modification and a 3′-wing segment comprising three nucleotides comprising a 2′OMe modification, wherein each internucleotide linkage of the agent is a phosphorothioate linkage. In some embodiments, each cytosine of the agent is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising three nucleotides comprising a 2′MOE modification and a 3′-wing segment comprising three nucleotides comprising a 2′MOE modification, wherein each internucleotide linkage of the agent is a phosphorothioate linkage. In some embodiments, each cytosine of the agent is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising three constrained ethyl nucleotides and a 3′-wing segment comprising three constrained ethyl nucleotides, wherein each internucleoitde linkage of the agent is a phosphorothioate linkage. In some embodiments, each cytosine of the agent is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising three LNA nucleotides and a 3′-wing segment comprising three LNA nucleotides, wherein each internucleotide linkage of the agent is a phosphorothioate linkage. In some embodiments, each cytosine of the agent is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising two nucleotides comprising a 2′OMe modification and a 3′-wing segment comprising two nucleotides comprising a 2′OMe modification, wherein each internucleotde linkage of the agent is a phosphorothioate linkage. In some embodiments, each cytosine of the agent is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising two nucleotides comprising a 2′MOE modification and a 3′-wing segment comprising two nucleotides comprising a 2′MOE modification, wherein each internucleotide linkage of the agent is a phosphorothioate linkage. In some embodiments, each cytosine of the agent is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising two constrained ethyl nucleotides and a 3′-wing segment comprising two constrained ethyl nucleotides, wherein each internucleoitde linkage of the agent is a phosphorothioate linkage. In some embodiments, each cytosine of the agent is a 5-methylcytosine.

In one embodiment, an antisense polynucleotide agent targeting an INHBE gene comprises a gap segment of ten 2′-deoxyribonucleotides positioned immediately adjacent to and between a 5′-wing segment comprising two LNA nucleotides and a 3′-wing segment comprising two LNA nucleotides, wherein each internucleotide linkage of the agent is a phosphorothioate linkage. In some embodiments, each cytosine of the agent is a 5-methylcytosine.

Further gapmer designs suitable for use in the agents, compositions, and methods of the invention are disclosed in, for example, U.S. Pat. Nos. 7,687,617 and 8,580,756; U.S. Patent Publication Nos. 20060128646, 20090209748, 20140128586, 20140128591, 20100210712, and 20080015162A1; and International Publication No. WO 2013/159108, the entire content of each of which are incorporated herein by reference.

### vi. Modulators Conjugated to Ligands

Another modification of the modulators, e.g., oligonucleotides, e.g., dsRNA agents, antisense polynucleotide agents, guideRNAs effecting ADAR editing or guideRNAs effecting CRISPR editing, of the invention involves chemically linking to the modulator to one or more ligands, moieties or conjugates that enhance the activity, cellular distribution, or cellular uptake of the oligonucleotide e.g., into a cell. Such moieties include but are not limited to lipid moieties such as a cholesterol moiety (Letsinger et al., Proc. Natl. Acid. Sci. USA, 1989, 86: 6553-6556). In other embodiments, the ligand is cholic acid (Manoharan et al., Biorg. Med. Chem. Let., 1994, 4:1053-1060), a thioether, e.g., beryl-S-tritylthiol (Manoharan et al., Ann. N. Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In certain embodiments, a ligand alters the distribution, targeting, or lifetime of an oligonucleotide into which it is incorporated. In some embodiments a ligand provides an enhanced affinity for a selected target, e.g., molecule, cell or cell type, compartment, e.g., a cellular or organ compartment, tissue, organ or region of the body, as, e.g., compared to a species absent such a ligand. In some embodiments, ligands do not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic. In certain embodiments, the ligand is a multivalent galactose, e.g., an N-acetyl-galactosamine.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the modulator, e.g., oligonucleotide, e.g., dsRNA agent or antisense polynucleotide agent, into the cell, for example, by disrupting the cell's cytoskeleton, e.g., by disrupting the cell's microtubules, microfilaments, or intermediate filaments. The drug can be, for example, taxol, vincristine, vinblastine, cytochalasin, nocodazole, japlakinolide, latrunculin A, phalloidin, swinholide A, indanocine, or myoservin.

In some embodiments, a ligand attached to an oligonucleotide as described herein acts as a pharmacokinetic modulator (PK modulator). PK modulators include lipophiles, bile acids, steroids, phospholipid analogues, peptides, protein binding agents, PEG, vitamins, etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin. Oligonucleotides that comprise a number of phosphorothioate linkages are also known to bind to serum protein, thus short oligonucleotides, e.g., oligonucleotides of about 5 bases, 10 bases, 15 bases, or 20 bases, comprising multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated oligonucleotides of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems® (Foster City, Calif.). Any other methods for such synthesis known in the art may additionally or alternatively be employed. It is also known to use similar techniques to prepare other oligonucleotides, such as the phosphorothioates and alkylated derivatives.

In the ligand-conjugated oligonucleotides and ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

### A. Lipid Conjugates

In certain embodiments, the ligand or conjugate is a lipid or lipid-based molecule. In one embodiment, such a lipid or lipid-based molecule binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, or (c) can be used to adjust binding to a serum protein, e.g., HSA.

A lipid based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In certain embodiments, the lipid based ligand binds HSA. In one embodiment, it binds HSA with a sufficient affinity such that the conjugate will be distributed to a non-kidney tissue. However, it is preferred that the affinity not be so strong that the HSA-ligand binding cannot be reversed.

In other embodiments, the lipid based ligand binds HSA weakly or not at all. In one embodiment, the conjugate will be distributed to the kidney. Other moieties that target to kidney cells can also be used in place of, or in addition to, the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by target cells such as liver cells. Also included are HSA and low density lipoprotein (LDL).

### B. Cell Permeation Agents

In another aspect, the ligand is a cell-permeation agent, such as, a helical cell-permeation agent. In one embodiment, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. In one embodiment, the helical agent is an alpha-helical agent, which has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 14). An RFGF analogue (e.g., amino acid sequence AALLPVLLAAP (SEQ ID NO:15) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO:16) and the Drosophila Antennapedia protein (RQIKIWFQNRRMKWKK (SEQ ID NO:17) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to a dsRNA agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidiomimemtics may include D-amino acids, as well as synthetic RGD mimics. In addition to RGD, one can use other moieties that target the integrin ligand, e.g., PECAM-1 or VEGF.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

### C. Carbohydrate Conjugates

In some embodiments of the compositions and methods of the invention, an oligonucleotide, e.g., dsRNA agent or antisense polynucleotide agent, further comprises a carbohydrate. The carbohydrate conjugated oligonucleotide is advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri-, and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In certain embodiments, a carbohydrate conjugate for use in the compositions and methods of the invention is a monosaccharide.

In certain embodiments, the monosaccharide is an N-acetylgalactosamine (GalNAc). GalNAc conjugates, which comprise one or more N-acetylgalactosamine (GalNAc) derivatives, are described, for example, in U.S. Pat. No. 8,106,022, the entire content of which is hereby incorporated herein by reference.

In some embodiments, the GalNAc conjugate serves as a ligand that targets the iRNA to particular cells. In some embodiments, the GalNAc conjugate targets the iRNA to liver cells, e.g., by serving as a ligand for the asialoglycoprotein receptor of liver cells (e.g., hepatocytes).

In some embodiments, the carbohydrate conjugate comprises one or more GalNAc derivatives. The GalNAc derivatives may be attached via a linker, e.g., a bivalent or trivalent branched linker. In some embodiments the GalNAc conjugate is conjugated to the 3′ end of the sense strand. In some embodiments, the GalNAc conjugate is conjugated to the oligonucleotide agent (e.g., to the 3′ end of the sense strand) via a linker, e.g., a linker as described herein. In some embodiments the GalNAc conjugate is conjugated to the 5′ end of the sense strand. In some embodiments, the GalNAc conjugate is conjugated to the iRNA agent (e.g., to the 5′ end of the sense strand) via a linker, e.g., a linker as described herein.

In certain embodiments of the invention, the GalNAc or GalNAc derivative is attached to an oligonucleotide of the invention via a monovalent linker. In some embodiments, the GalNAc or GalNAc derivative is attached to an oligonucleotide of the invention via a bivalent linker. In yet other embodiments of the invention, the GalNAc or GalNAc derivative is attached to an oligonucleotide of the invention via a trivalent linker. In other embodiments of the invention, the GalNAc or GalNAc derivative is attached to an oligonucleotide of the invention via a tetravalent linker.

In certain embodiments, the oligonucleotides of the invention comprise one GalNAc or GalNAc derivative attached to the oligonucleotide. In certain embodiments, the oligonucleotides of the invention comprise a plurality (e.g., 2, 3, 4, 5, or 6) GalNAc or GalNAc derivatives, each independently attached to a plurality of nucleotides of the oligonucleotide through a plurality of monovalent linkers.

In some embodiments, for example, when the two strands of an iRNA agent of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker. The hairpin loop may also be formed by an extended overhang in one strand of the duplex.

In some embodiments, for example, when the two strands of an iRNA agent of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker. The hairpin loop may also be formed by an extended overhang in one strand of the duplex.

In one embodiment, a carbohydrate conjugate for use in the compositions and methods of the invention is selected from the group consisting of:

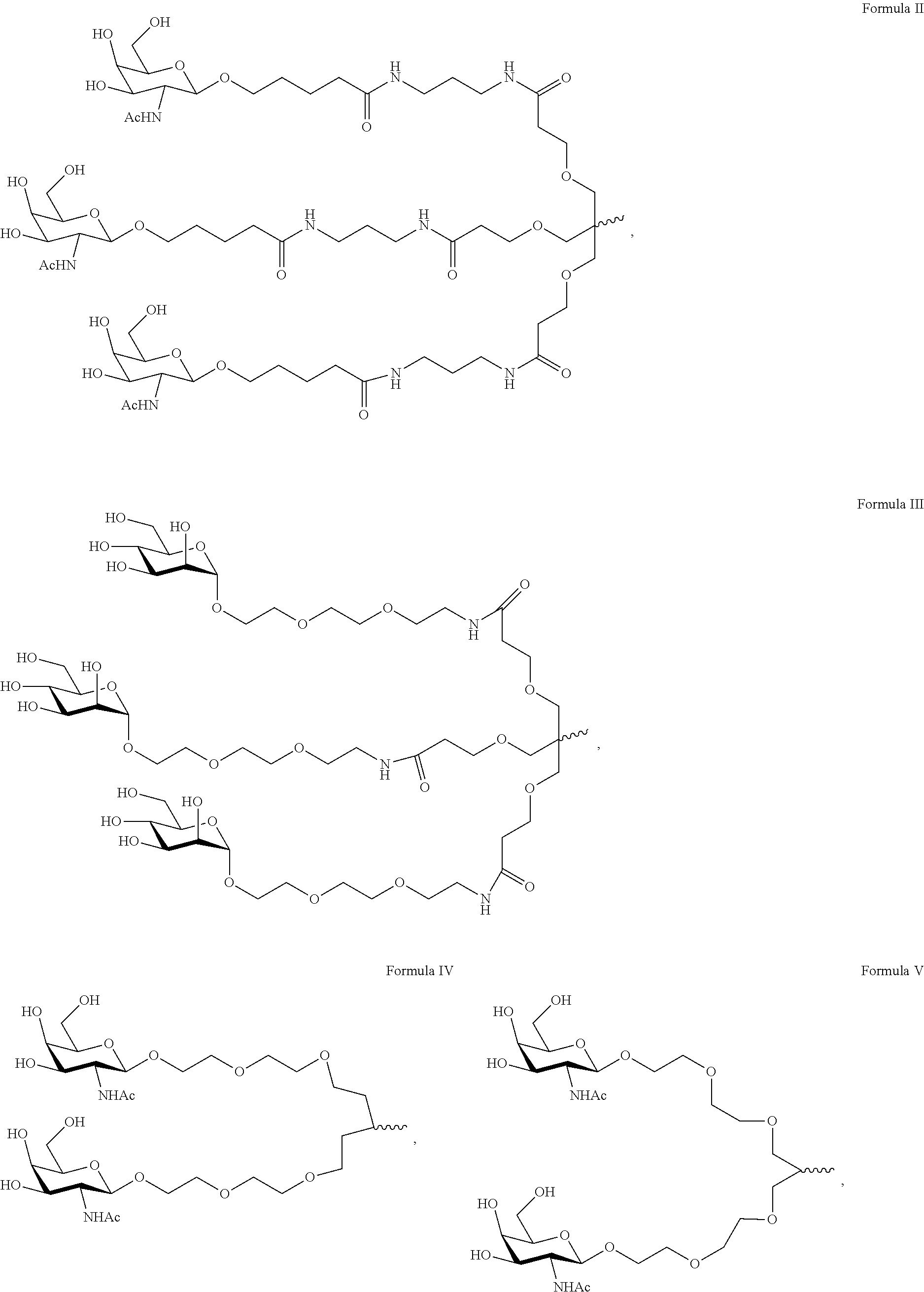

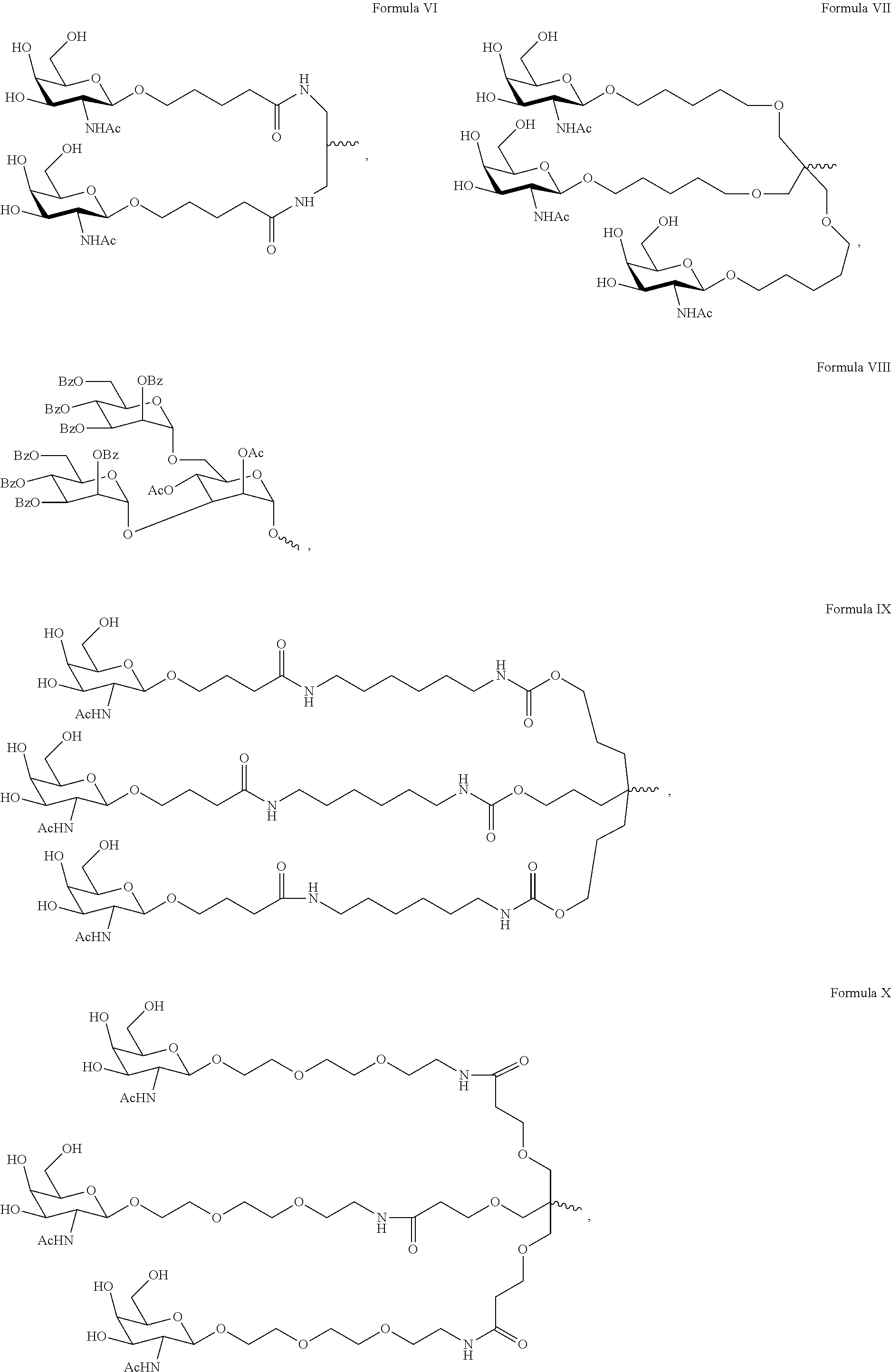

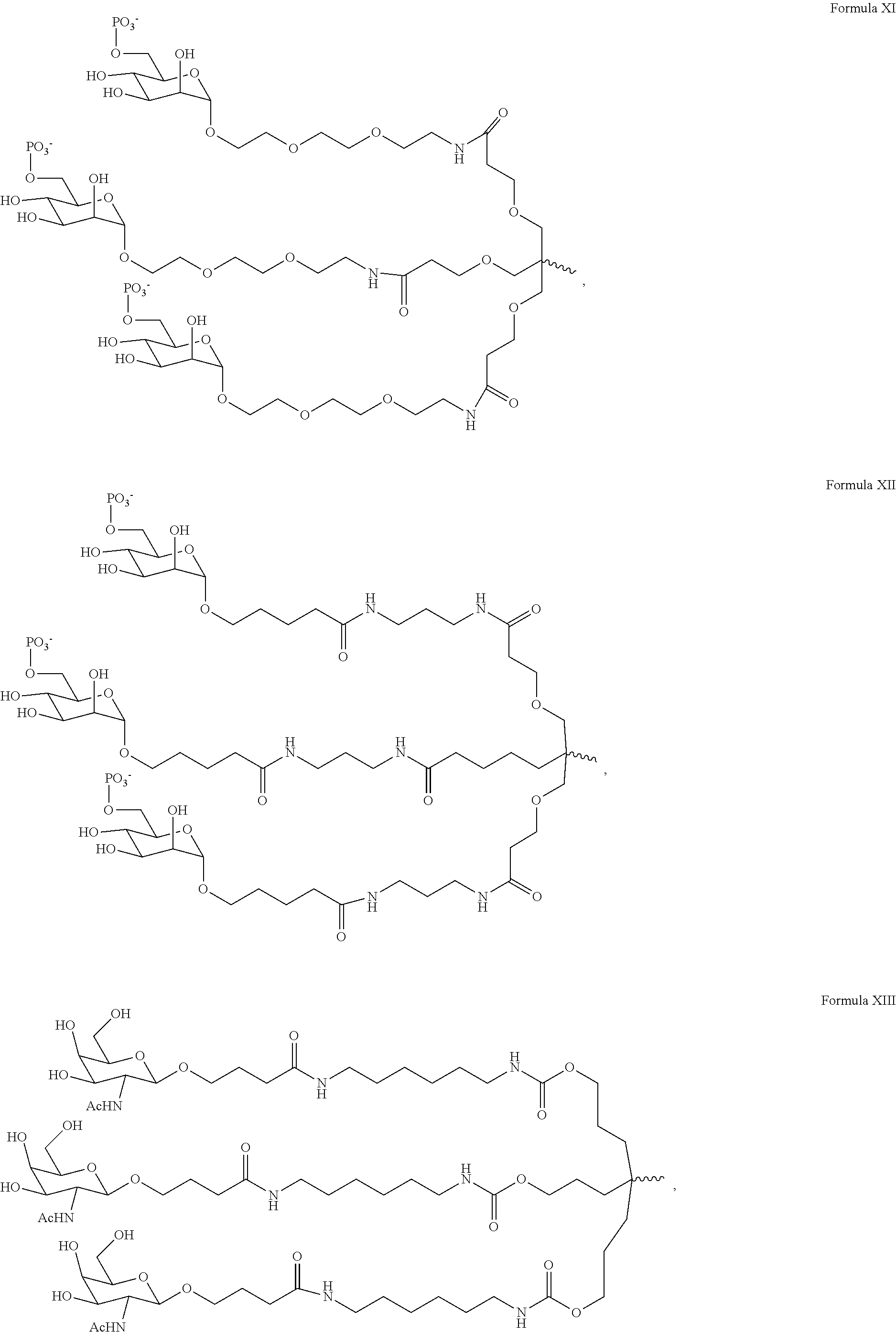

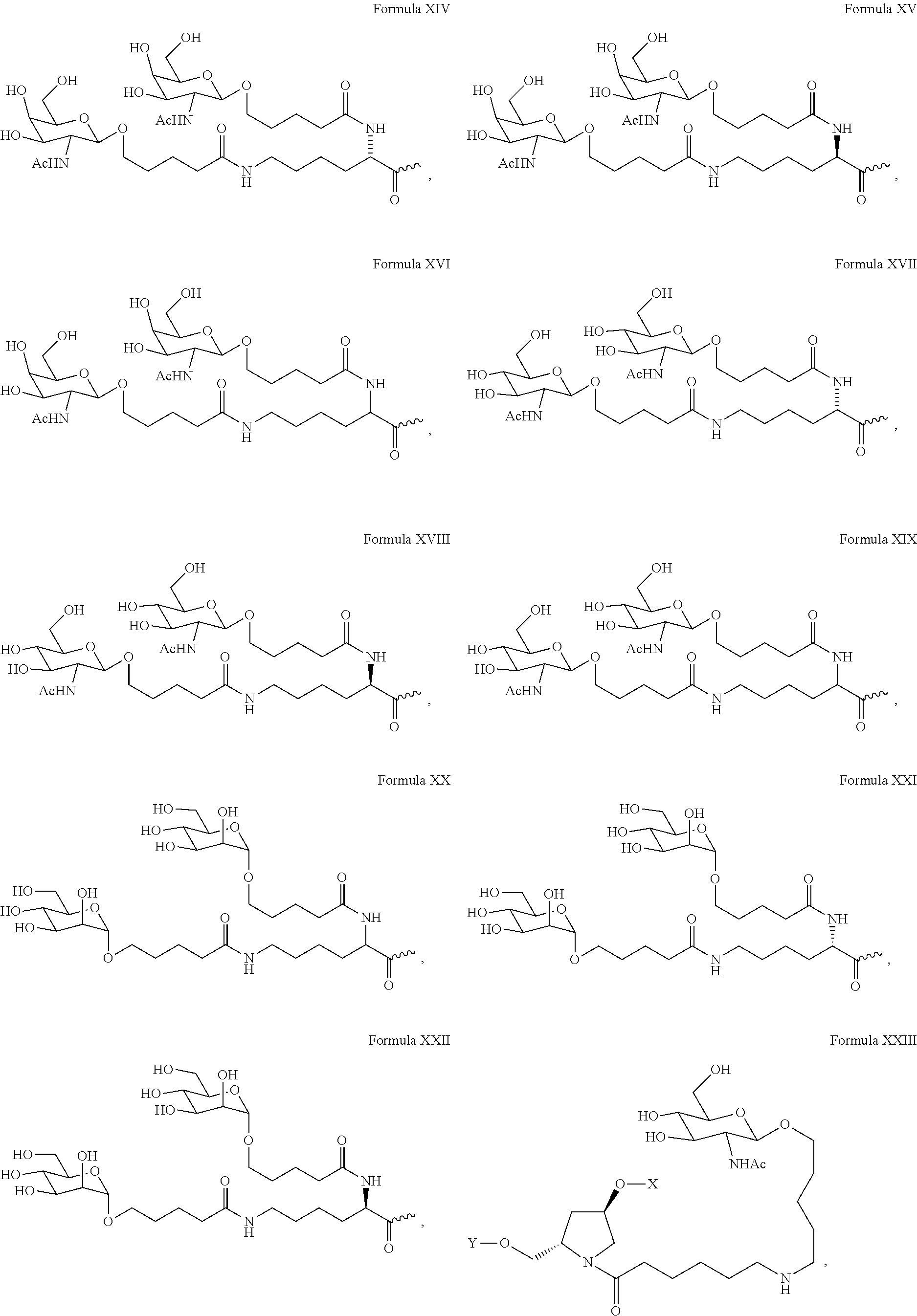

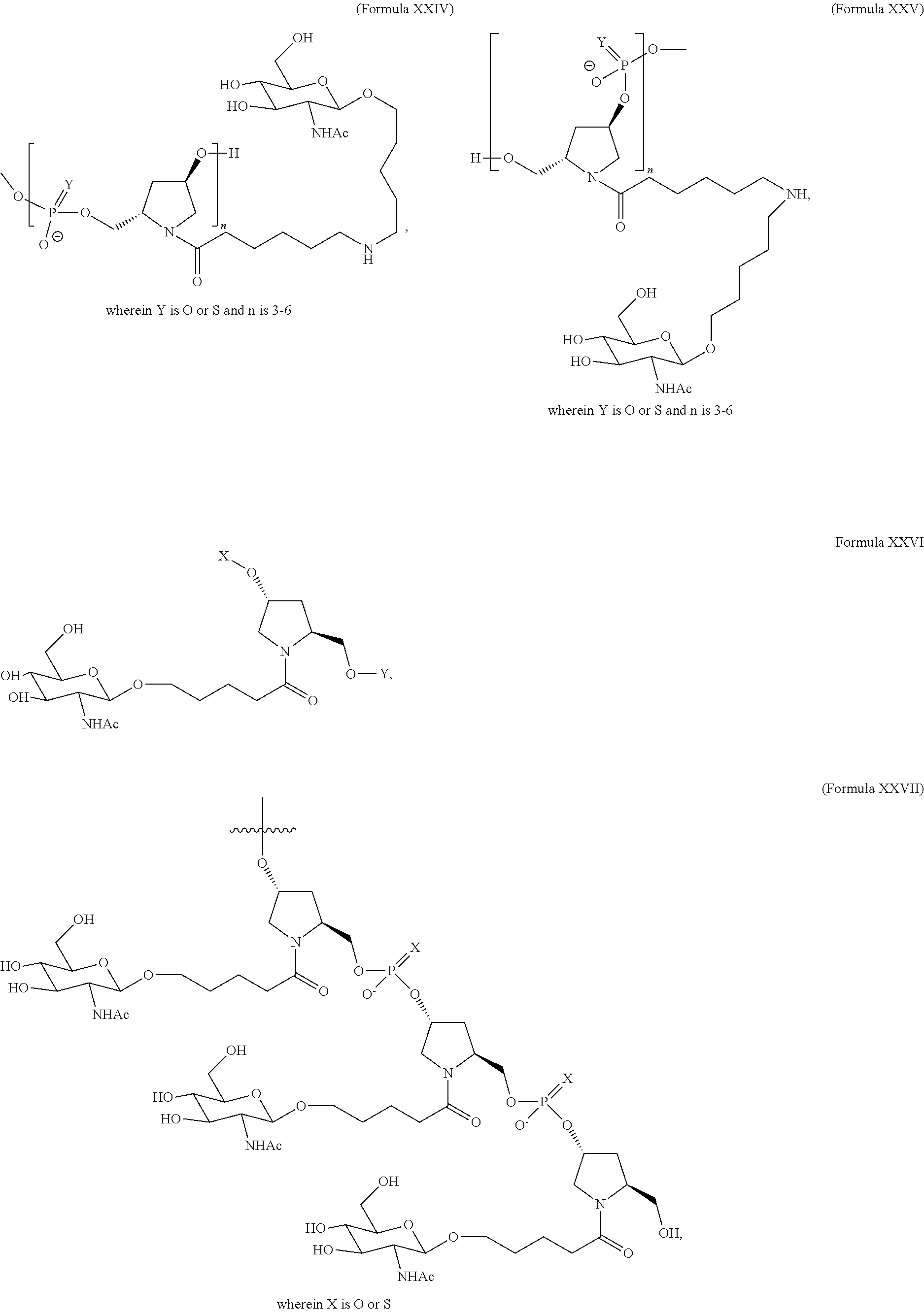

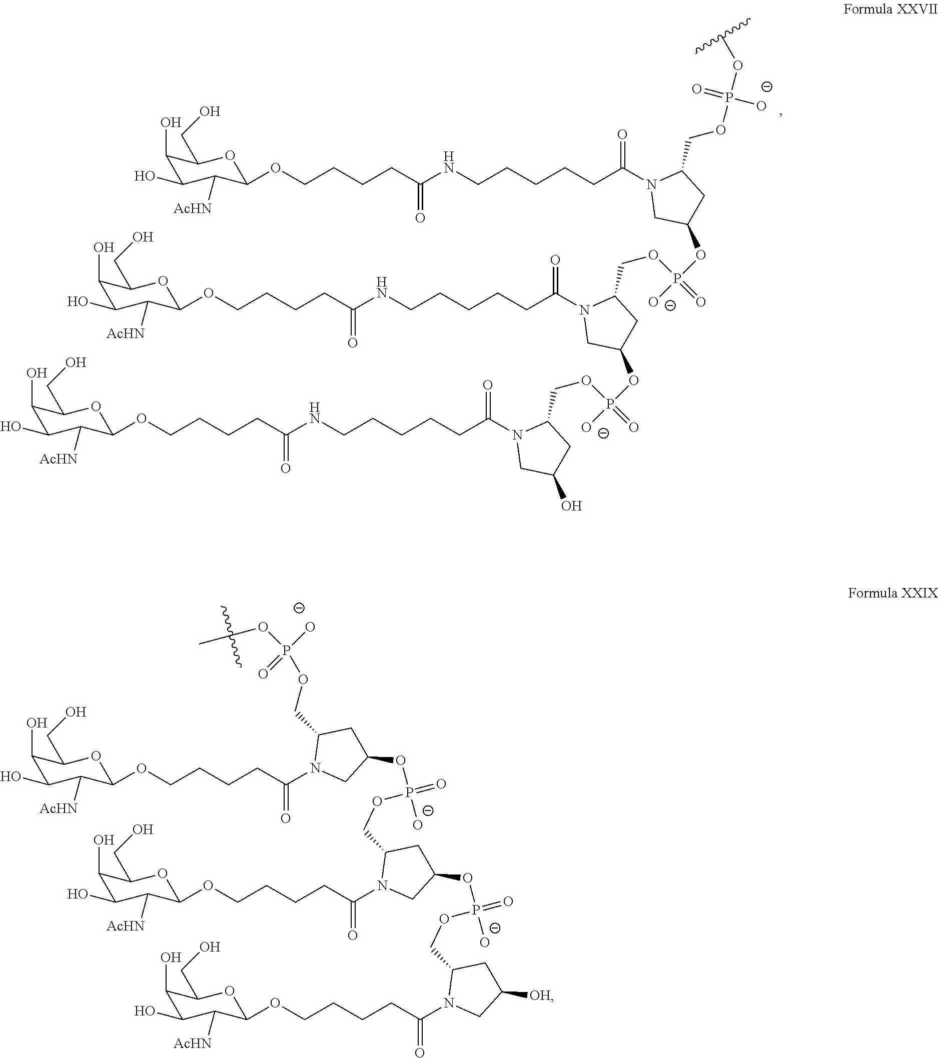 [SMILES: *OP(=O)([O-])OC[C@@H]1C[C@@H](OP(=O)([O-])OC[C@@H]2C[C@@H](OP(=O)([O-])OC[C@@H]3C[C@@H](O)CN3C(=O)CCCCCNC(=O)CCCCOC3OC(CO)C(O)C(O)C3NC(C)=O)CN2C(=O)CCCCCNC(=O)CCCCOC2OC(CO)C(O)C(O)C2NC(C)=O)CN1C(=O)CCCCCNC(=O)CCCCOC1OC(CO)C(O)C(O)C1NC(C)=O ; *OP(=O)([O-])OC[C@@H]1C[C@@H](OP(=O)([O-])OC[C@@H]2C[C@@H](OP(=O)([O-])OC[C@@H]3C[C@@H](O)CN3C(=O)CCCCOC3OC(CO)C(O)C(O)C3NC(C)=O)CN2C(=O)CCCCOC2OC(CO)C(O)C(O)C2NC(C)=O)CN1C(=O)CCCCOC1OC(CO)C(O)C(O)C1NC(C)=O]

In another embodiment, a carbohydrate conjugate for use in the compositions and methods of the invention is a monosaccharide. In one embodiment, the monosaccharide is an N-acetylgalactosamine, such as

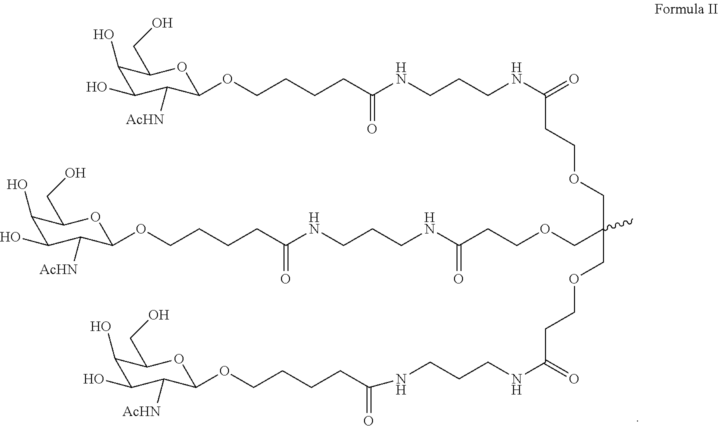 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the oligonucleotide is attached to the carbohydrate conjugate via a linker as shown in the following schematic, wherein X is O or S.

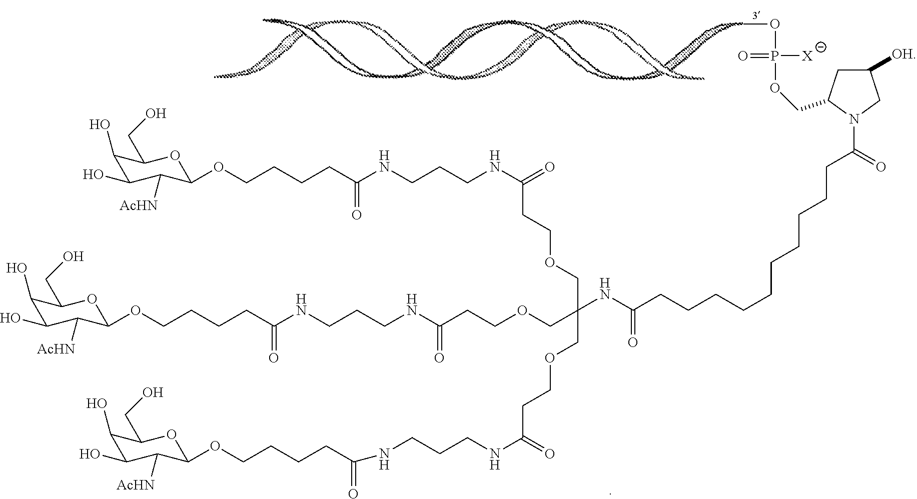

In some embodiments, the oligonucleotide is conjugated to L96 as defined in Table 1 and shown below:

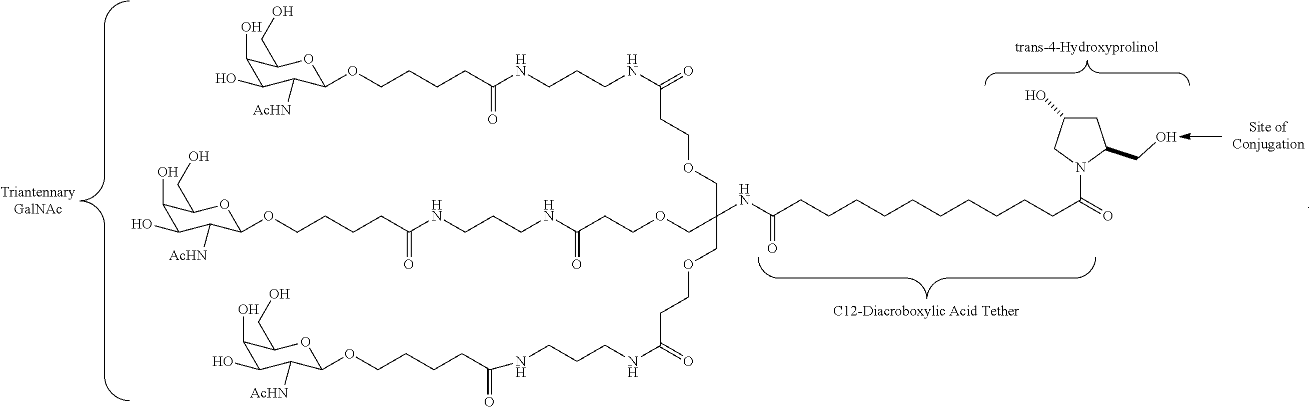

Another representative carbohydrate conjugate for use in the embodiments described herein includes, but is not limited to,

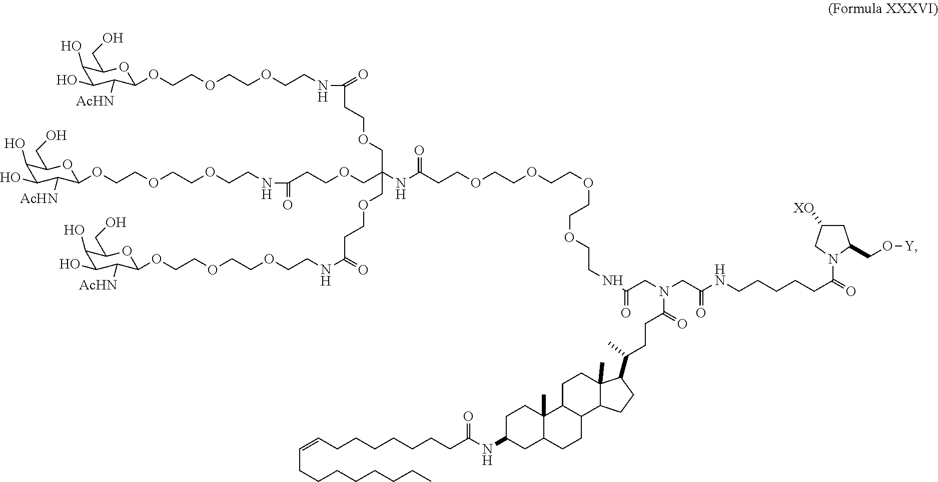

when one of X or Y is an oligonucleotide, the other is a hydrogen.

In some embodiments, a suitable ligand is a ligand disclosed in WO 2019/055633, the entire contents of which are incorporated herein by reference. In one embodiment the ligand comprises the structure below:

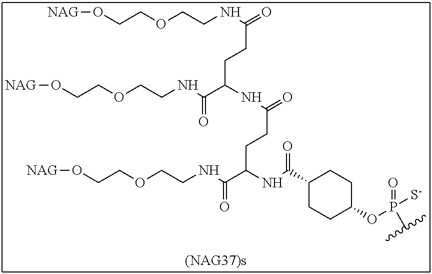

In certain embodiments of the invention, the GalNAc or GalNAc derivative is attached to an oligonucleotide of the invention via a monovalent linker. In some embodiments, the GalNAc or GalNAc derivative is attached to an oligonucleotide of the invention via a bivalent linker. In yet other embodiments of the invention, the GalNAc or GalNAc derivative is attached to an oligonucleotide of the invention via a trivalent linker.

In one embodiment, the oligonucleotides of the invention comprise one or more GalNAc or GalNAc derivative attached to the oligonucleotide. The GalNAc may be attached to any nucleotide via a linker on the sense strand or antsisense strand. The GalNac may be attached to the 5′-end of the sense strand, the 3′ end of the sense strand, the 5′-end of the antisense strand, or the 3′-end of the antisense strand. In one embodiment, the GalNAc is attached to the 3′ end of the sense strand, e.g., via a trivalent linker.

In other embodiments, the oligonucleotides of the invention comprise a plurality (e.g., 2, 3, 4, 5, or 6) GalNAc or GalNAc derivatives, each independently attached to a plurality of nucleotides of the oligonucleotide through a plurality of linkers, e.g., monovalent linkers.

In some embodiments, for example, when the two strands of an iRNA agent of the invention is part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker.

In some embodiments, the carbohydrate conjugate further comprises one or more additional ligands as described above, such as, but not limited to, a PK modulator or a cell permeation peptide.

Additional carbohydrate conjugates and linkers suitable for use in the present invention include those described in PCT Publication Nos. WO 2014/179620 and WO 2014/179627, the entire contents of each of which are incorporated herein by reference.

### D. Linkers

In some embodiments, the conjugate or ligand described herein can be attached to an oligonucleotide with various linkers that can be cleavable or non-cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, or substituted or unsubstituted heterocyclic; where R8 is hydrogen, acyl, aliphatic, or substituted aliphatic. In one embodiment, the linker is about 1-24 atoms, 2-24, 3-24, 4-24, 5-24, 6-24, 6-18, 7-18, 8-18, 7-17, 8-17, 6-16, 7-17, or 8-16 atoms.

A cleavable linking group is one which is sufficiently stable outside the cell, but which upon entry into a target cell is cleaved to release the two parts the linker is holding together. In an exemplary embodiment, the cleavable linking group is cleaved at least about 10 times, 20, times, 30 times, 40 times, 50 times, 60 times, 70 times, 80 times, 90 times, or more, or at least 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a selected pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In certain embodiments, useful candidate compounds are cleaved at least about 2, 4, 10, 20, 30, 40, 50, 60, 70, 80, 90, or 100 times faster in the cell (or under in vitro conditions selected to mimic intracellular conditions) as compared to blood or serum (or under in vitro conditions selected to mimic extracellular conditions).

i. Redox Cleavable Linking Groups

In certain embodiments, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular oligonucleotide and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

ii. Phosphate-Based Cleavable Linking Groups

In other embodiments, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—, wherein Rk at each occurrence can be, independently, C1-C20 alkyl, C1-C20 haloalkyl, C6-C10 aryl, or C7-C12 aralkyl. Exemplary embodiments include —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O, —S—P(S)(H)—O—, —S—P(O)(H)—S—, and —O—P(S)(H)—S—. In certain embodiments a phosphate-based linking group is —O—P(O)(OH)—O—. These candidates can be evaluated using methods analogous to those described above.

iii. Acid Cleavable Linking Groups

In other embodiments, a cleavable linker comprises an acid cleavable linking group. An acid cleavable linking group is a linking group that is cleaved under acidic conditions. In certain embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). An exemplary embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

iv. Ester-Based Linking Groups

In other embodiments, a cleavable linker comprises an ester-based cleavable linking group. An ester-based cleavable linking group is cleaved by enzymes such as esterases and amidases in cells. Examples of ester-based cleavable linking groups include, but are not limited to, esters of alkylene, alkenylene and alkynylene groups. Ester cleavable linking groups have the general formula —C(O)O—, or —OC(O)—. These candidates can be evaluated using methods analogous to those described above.

v. Peptide-Based Cleaving Groups

In yet other embodiments, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

In some embodiments, an iRNA of the invention is conjugated to a carbohydrate through a linker. Non-limiting examples of iRNA carbohydrate conjugates with linkers of the compositions and methods of the invention include, but are not limited to,

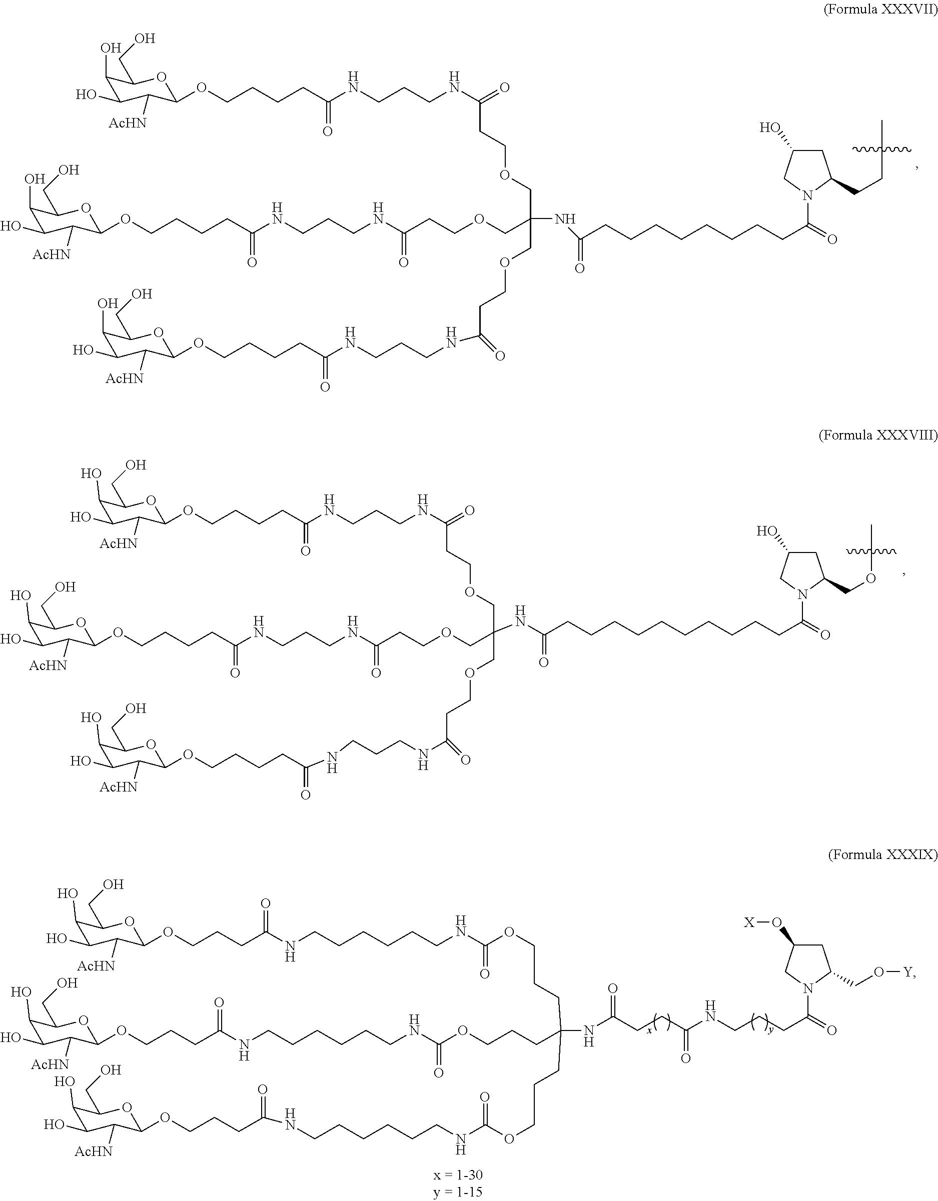

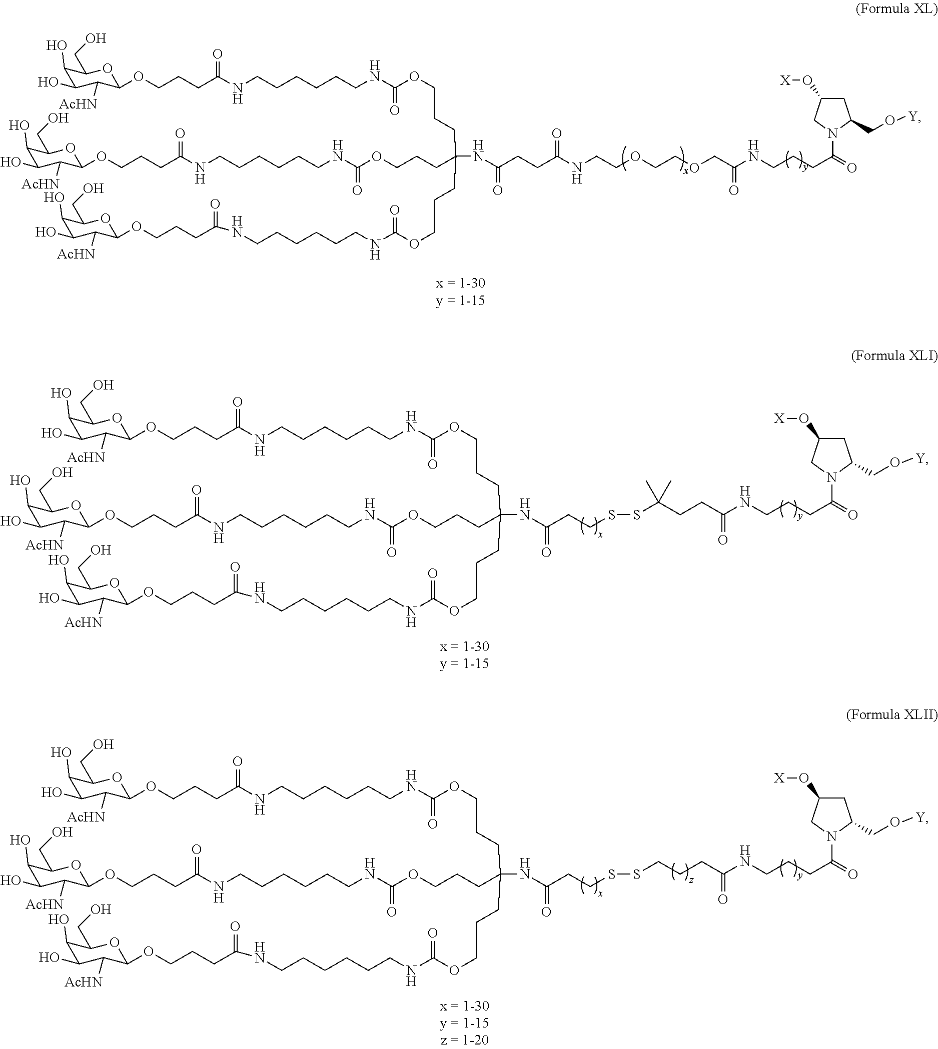

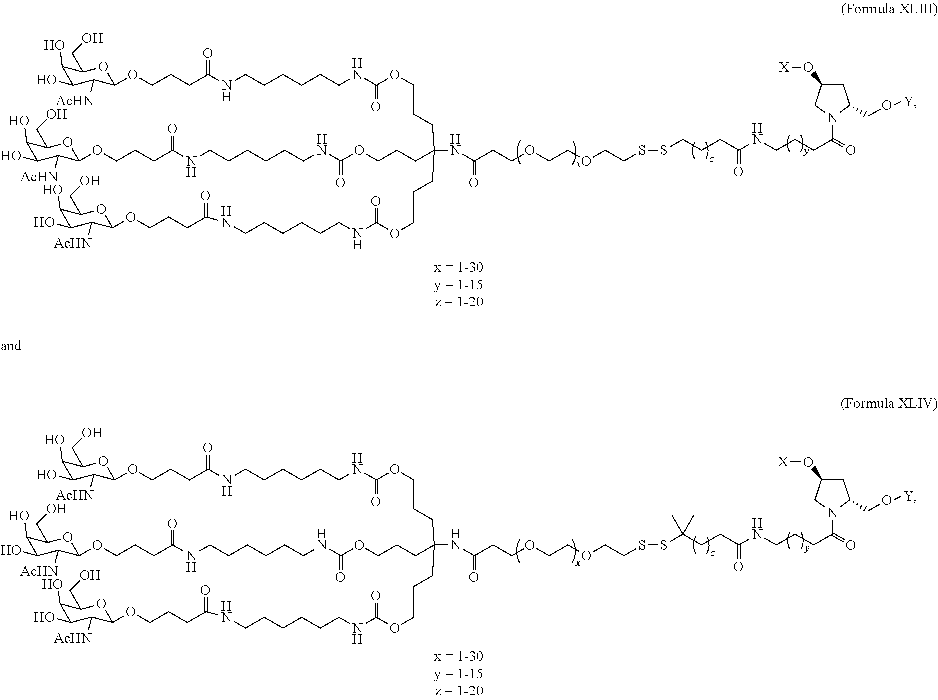

when one of X or Y is an oligonucleotide, the other is a hydrogen.

In certain embodiments of the compositions and methods of the invention, a ligand is one or more “GalNAC” (N-acetylgalactosamine) derivatives attached through a bivalent or trivalent branched linker.

In one embodiment, an oligonucleotide of the invention is conjugated to a bivalent or trivalent branched linker selected from the group of structures shown in any of formula (XLV)-(XLVI):

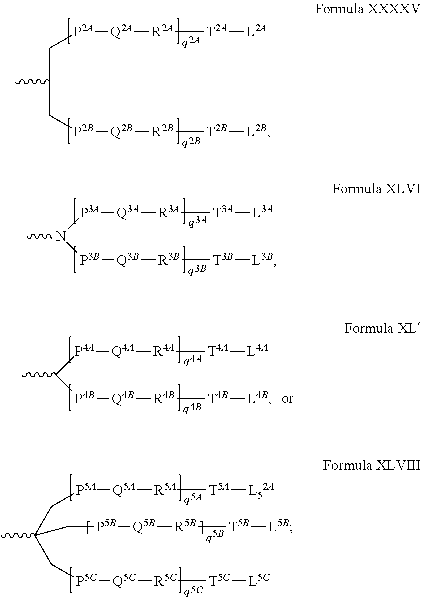

wherein:
q2A, q2B, q3A, q3B, q4A, q4B, q5A, q5B and q5C represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A} Q^{5B}, Q^{5C} are independently for each occurrence absent, alkylene, substituted alkylene wherein one or more methylenes can be interrupted or terminated by one or more of O, S, S(O), SO₂, N(R^{N}), C(R′)═C(R″), C≡C or C(O);
R^{2A}, R^{2B}, R^{3A}, R^{3A}, R^{4A}, R^{4B}, R^{5A}, R^{5B}, R^{5C} are each independently for each occurrence absent. NH, O, S, CH₂, C(O)O, C(O)NH, NHCH(R^{a})C(O), —C(O)—CH(R^{a})—NH—, CO, CH═N—O,

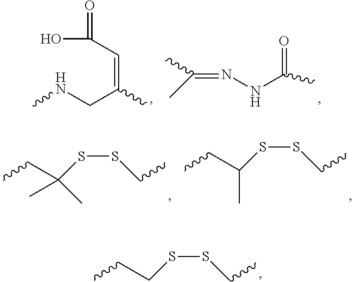

or heterocyclyl;
L^{2A}, L^{2B}, L^{3A}, L^{3B}, L^{4A}, L^{4B} L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with RNAi agents for inhibiting the expression of a target gene, such as those of formula (XLIX):

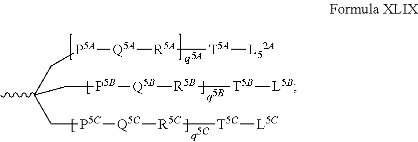

wherein L^{5A}, L^{5B} and L^{5C} represent a monosacharide, such as GalNAc derivative.

Examples of suitable bivalent and trivalent branched linker groups conjugating GalNAc derivatives include, but are not limited to, the structures recited above as formulas II, VII, XI, X, and XIII.

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,688,941; 6,294,664; 6,320,017; 6,576,752; 6,783,931; 6,900,297; 7,037,646; and 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an oligonucleotide. The present invention also includes oligonucleotide compounds that are chimeric compounds.

For example, “chimeric” iRNA compounds or “chimeras,” in the context of this invention, are iRNA compounds, such as, dsRNAi agents, that contain two or more chemically distinct regions, each made up of at least one monomer unit, i.e., a nucleotide in the case of a dsRNA compound. These iRNAs typically contain at least one region wherein the RNA is modified so as to confer upon the iRNA increased resistance to nuclease degradation, increased cellular uptake, or increased binding affinity for the target nucleic acid. An additional region of the iRNA can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the oligonucleotide can be modified by a non-ligand group. A number of non-ligand molecules have been conjugated to oligonucleotides in order to enhance the activity, cellular distribution or cellular uptake of the oligonucleotide, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such oligonucleotide conjugates have been listed above. Typical conjugation protocols involve the synthesis of oligonucleotides bearing an aminolinker at one or more positions of the sequence. The amino group is then reacted with the molecule being conjugated using appropriate coupling or activating reagents. The conjugation reaction can be performed either with the oligonucleotide still bound to the solid support or following cleavage of the oligonucleotide, in solution phase. Purification of the RNA conjugate by HPLC typically affords the pure conjugate.

### B. Antibodies, or Antigen-Binding Fragments Thereof, of the Invention

In some embodiments, the modulator of the invention is an antibody, or antigen-binding fragment thereof, that specifically binds INHBE, e.g., a monoclonal anti-INHBE antibody, or antigen-binding fragment thereof.

The antibody modulators can be identified, screened for (e.g., using phage display), or characterized for their physical/chemical properties and/or biological activities by various assays known in the art (see, for example, Antibodies: A Laboratory Manual, Second edition, Greenfield, ed., 2014). Binding specificity of an antibody for its antigen can be tested by known methods in the art such as ELISA, Western blot, or surface plasmon resonance.

In some embodiments, the anti-INHBE antibody or antigen-binding fragment thereof is a humanized antibody or antigen-binding fragment thereof. Humanized antibodies may be useful as therapeutic molecules because humanized antibodies may reduce or eliminate the human immune response to non-human antibodies (such as the human anti-mouse antibody response), which can result in an immune response to an antibody therapeutic, and decreased effectiveness of the therapeutic.

In some embodiments, the anti-INHBE antibody or antigen-binding fragment thereof is a chimeric antibody or antigen-binding fragment thereof. In some embodiments, an anti-INHBE antibody or antigen-binding fragment thereof comprises at least one non-human variable region and at least one human constant region. In some such embodiments, all of the variable regions of an anti-INHBE antibody are non-human variable regions, and all of the constant regions of an anti-INHBE antibody are human constant regions. In some embodiments, one or more variable regions of a chimeric antibody are mouse variable regions. The human constant region of a chimeric antibody need not be of the same isotype as the non-human constant region, if any, it replaces. Chimeric antibodies are discussed, e.g., in U.S. Pat. No. 4,816,567; and Morrison et al. Proc. Natl. Acad. Sci. USA 81: 6851-55 (1984).

In some embodiments, the anti-INHBE antibody or antigen-binding fragment thereof is a human antibody or antigen-binding fragment thereof.

In some embodiments, the antibody modulator, e.g., the anti-INHBE antibody or antigen-binding fragment thereof, is a monoclonal anti-INHBE antibody or antigen-binding fragment thereof.

In some embodiments, the antibody modulator, e.g., the anti-INHBE antibody or antigen-binding fragment thereof, is multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present invention using routine techniques available in the art.

The antibody modulators of the present invention can be produced using any methods known in the art. For example, the antibodies, and antigen-binding fragments thereof, can be produced using recombinant DNA methods. Expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

Host cells may be a prokaryotic or eukaryotic cell. The polynucleotide or vector which is present in the host cell may either be integrated into the genome of the host cell or it may be maintained extrachromosomally. The host cell can be any prokaryotic or eukaryotic cell, such as a bacterial, insect, fungal, plant, animal or human cell. In some embodiments, fungal cells are, for example, those of the genus Saccharomyces, in particular those of the species S. cerevisiae. The term “prokaryotic” includes all bacteria which can be transformed or transfected with a DNA or RNA molecules for the expression of an antibody or the corresponding immunoglobulin chains. Prokaryotic hosts may include gram negative as well as gram positive bacteria such as, for example, E. coli, S. typhimurium, Serratia marcescens and Bacillus subtilis. The term “eukaryotic” includes yeast, higher plants, insects and vertebrate cells, e.g., mammalian cells, such as NSO and CHO cells. Depending upon the host employed in a recombinant production procedure, the antibodies or immunoglobulin chains encoded by the polynucleotide may be glycosylated or may be non-glycosylated. Antibodies or the corresponding immunoglobulin chains may also include an initial methionine amino acid residue. Although it is possible to express antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

In some embodiments, once a vector has been incorporated into an appropriate host, the host may be maintained under conditions suitable for high level expression of the nucleotide sequences, and, as desired, the collection and purification of the immunoglobulin light chains, heavy chains, light/heavy chain dimers or intact antibodies, antigen binding fragments thereof or other immunoglobulin forms may follow; see, Beychok, Cells of Immunoglobulin Synthesis, Academic Press, N.Y., (1979). Thus, polynucleotides or vectors are introduced into the cells which in turn produce the antibody or antigen binding fragments thereof. Furthermore, transgenic animals, preferably mammals, comprising the aforementioned host cells may be used for the large scale production of the antibody or antibody fragments thereof.

The transformed host cells can be grown in fermenters and cultured using any suitable techniques to achieve optimal cell growth. Once expressed, the whole antibodies, their dimers, individual light and heavy chains, other immunoglobulin forms, or antigen binding fragments thereof, can be purified according to standard procedures of the art, including ammonium sulfate precipitation, affinity columns, column chromatography, gel electrophoresis and the like; see, Scopes, “Protein Purification”, Springer Verlag, N.Y. (1982). The antibody or antigen binding fragments thereof can then be isolated from the growth medium, cellular lysates, or cellular membrane fractions. The isolation and purification of the, e.g., microbially expressed antibodies or antigen binding fragments thereof may be by any conventional means such as, for example, preparative chromatographic separations and immunological separations such as those involving the use of monoclonal or polyclonal antibodies directed, e.g., against the constant region of the antibody.

Aspects of the present invention relate to a hybridoma, which provides an indefinitely prolonged source of monoclonal antibodies. As an alternative to obtaining immunoglobulins directly from the culture of hybridomas, immortalized hybridoma cells can be used as a source of rearranged heavy chain and light chain loci for subsequent expression and/or genetic manipulation. Rearranged antibody genes can be reverse transcribed from appropriate mRNAs to produce cDNA. In some embodiments, heavy chain constant region can be exchanged for that of a different isotype or eliminated altogether. The variable regions can be linked to encode single chain Fv regions. Multiple Fv regions can be linked to confer binding ability to more than one target or chimeric heavy and light chain combinations can be employed. Any appropriate method may be used for cloning of antibody variable regions and generation of recombinant antibodies, and antigen-binding portions thereof.

In some embodiments, an appropriate nucleic acid that encodes variable regions of a heavy and/or light chain is obtained and inserted into an expression vectors which can be transfected into standard recombinant host cells. A variety of such host cells may be used. In some embodiments, mammalian host cells may be advantageous for efficient processing and production. Typical mammalian cell lines useful for this purpose include CHO cells, 293 cells, or NSO cells. The production of the antibody or antigen binding fragment thereof may be undertaken by culturing a modified recombinant host under culture conditions appropriate for the growth of the host cells and the expression of the coding sequences. The antibodies or antigen binding fragments thereof may be recovered by isolating them from the culture. The expression systems may be designed to include signal peptides so that the resulting antibodies are secreted into the medium; however, intracellular production is also possible.

The present invention also includes a polynucleotide encoding at least a variable region of an immunoglobulin chain of the antibodies described herein. In some embodiments, the variable region encoded by the polynucleotide comprises at least one complementarity determining region (CDR) of the VH and/or VL of the variable region of the antibody produced by any one of the above described hybridomas.

Polynucleotides encoding antibody or antigen binding fragments thereof may be, e.g., DNA, cDNA, RNA or synthetically produced DNA or RNA or a recombinantly produced chimeric nucleic acid molecule comprising any of those polynucleotides either alone or in combination. In some embodiments, a polynucleotide is part of a vector. Such vectors may comprise further genes such as marker genes which allow for the selection of the vector in a suitable host cell and under suitable conditions.

In some embodiments, a polynucleotide is operatively linked to expression control sequences allowing expression in prokaryotic or eukaryotic cells. Expression of the polynucleotide comprises transcription of the polynucleotide into a translatable mRNA. Regulatory elements ensuring expression in eukaryotic cells, preferably mammalian cells, are well known to those skilled in the art. They may include regulatory sequences that facilitate initiation of transcription and optionally poly-A signals that facilitate termination of transcription and stabilization of the transcript. Additional regulatory elements may include transcriptional as well as translational enhancers, and/or naturally associated or heterologous promoter regions. Possible regulatory elements permitting expression in prokaryotic host cells include, e.g., the PL, Lac, Trp or Tac promoter in E. coli, and examples of regulatory elements permitting expression in eukaryotic host cells are the AOX1 or GAL1 promoter in yeast or the CMV-promoter, SV40-promoter, RSV-promoter (Rous sarcoma virus), CMV-enhancer, SV40-enhancer or a globin intron in mammalian and other animal cells.

Beside elements which are responsible for the initiation of transcription such regulatory elements may also include transcription termination signals, such as the SV40-poly-A site or the tk-poly-A site, downstream of the polynucleotide. Furthermore, depending on the expression system employed, leader sequences capable of directing the polypeptide to a cellular compartment or secreting it into the medium may be added to the coding sequence of the polynucleotide and have been described previously. The leader sequence(s) is (are) assembled in appropriate phase with translation, initiation and termination sequences, and preferably, a leader sequence capable of directing secretion of translated protein, or a portion thereof, into, for example, the extracellular medium. Optionally, a heterologous polynucleotide sequence can be used that encode a fusion protein including a C- or N-terminal identification peptide imparting desired characteristics, e.g., stabilization or simplified purification of expressed recombinant product.

In some embodiments, polynucleotides encoding at least the variable domain of the light and/or heavy chain may encode the variable domains of both immunoglobulin chains or only one. Likewise, a polynucleotide(s) may be under the control of the same promoter or may be separately controlled for expression. Furthermore, some aspects relate to vectors, particularly plasmids, cosmids, viruses and bacteriophages used conventionally in genetic engineering that comprise a polynucleotide encoding a variable domain of an immunoglobulin chain of an antibody or antigen binding fragment thereof; optionally in combination with a polynucleotide that encodes the variable domain of the other immunoglobulin chain of the antibody.

In some embodiments, expression control sequences are provided as eukaryotic promoter systems in vectors capable of transforming or transfecting eukaryotic host cells, but control sequences for prokaryotic hosts may also be used. Expression vectors derived from viruses such as retroviruses, vaccinia virus, adeno-associated virus, herpes viruses, or bovine papilloma virus, may be used for delivery of the polynucleotides or vector into targeted cell population (e.g., to engineer a cell to express an antibody or antigen binding fragment thereof). A variety of appropriate methods can be used to construct recombinant viral vectors. In some embodiments, polynucleotides and vectors can be reconstituted into liposomes for delivery to target cells. The vectors containing the polynucleotides (e.g., the heavy and/or light variable domain(s) of the immunoglobulin chains encoding sequences and expression control sequences) can be transferred into the host cell by suitable methods, which vary depending on the type of cellular host.

Monoclonal antibodies, and antigen-binding fragments thereof, may also be produced by generation of hybridomas (see e.g., Kohler and Milstein (1975) Nature, 256: 495-499) in accordance with known methods. Hybridomas formed in this manner are then screened using standard methods, such as enzyme-linked immunosorbent assay (ELISA) and surface plasmon resonance (e.g., OCTET or BIACORE) analysis, to identify one or more hybridomas that produce an antibody, or an antigen-binding portion thereof, that specifically binds to a specified antigen, e.g., INHBE, e.g., wild type INHBE, or mutant INHBE. Any form of the specified antigen may be used as the immunogen, e.g., recombinant antigen, naturally occurring forms, any variants or fragments thereof, as well as antigenic peptide thereof (e.g., any of the epitopes described herein as a linear epitope or within a scaffold as a conformational epitope). One exemplary method of making antibodies, and antigen-binding portions thereof, includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228:1315-1317; Clackson et al. (1991) Nature, 352: 624-628; Marks et al. (1991) J. Mol. Biol., 222: 581-597WO92/18619; WO 91/17271; WO 92/20791; WO 92/15679; WO 93/01288; WO 92/01047; WO 92/09690; and WO 90/02809.

In addition to the use of display libraries, the specified antigen (e.g., INHBE) can be used to immunize a non-human animal, e.g., a rodent, e.g., a mouse, hamster, or rat. In one embodiment, the non-human animal is a mouse.

In another embodiment, a monoclonal antibody is obtained from the non-human animal, and then modified, e.g., chimeric, using suitable recombinant DNA techniques. A variety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397.

For additional antibody production techniques, see Antibodies: A Laboratory Manual, eds. Harlow et al., Cold Spring Harbor Laboratory, 1988. The present invention is not necessarily limited to any particular source, method of production, or other special characteristics of an antibody.

Methods for generating human antibodies in transgenic mice are also known in the art. Any such known methods can be used in the context of the present invention to make human antibodies that specifically bind to human INHBE.

In some embodiment, high affinity chimeric antibodies are isolated having a human variable region and a mouse constant region. The antibodies are characterized and selected for desirable characteristics, including affinity, selectivity, epitope, etc. The mouse constant regions are replaced with a desired human constant region to generate the fully human antibody of the invention, for example wild-type or modified lgG1 or lgG4. While the constant region selected may vary according to specific use, high affinity antigen-binding and target specificity characteristics reside in the variable region.

### C. GuideRNAs Effecting ADAR Editing of the Invention

The present invention also provides guideRNAs that effect ADAR editing of the INHBE gene. Any of the nucleotides disclosed herein (such as the nucleotides in Tables 2-5) can be used to design guideRNAs that effect ADAR editing. Methods for designing and preparing such guideRNAs are described in, for example, WO2016097212A1, WO2017220751A1, US20210261955A1 and WO2018041973A1, the entire contents of which are incorporated herein by reference.

### D. GuideRNAs Effecting CRISPR Editing of the Invention

The present invention also provides guideRNAs that effect CRISPR editing of the INHBE gene. Any of the nucleotides disclosed herein (such as the nucleotides in Tables 2-5) can be used to design guideRNAs that effect CRISPR editing. Methods for designing and preparing such guideRNAs are described in, for example, US20200248180 and US20190316121, the entire contents of which are incorporated herein by reference.

### IV. Delivery of a Modulator of the Invention

The delivery of a modulator of the invention to a cell e.g., a cell within a subject, such as a human subject (e.g., a subject in need thereof, such as a subject susceptible to or diagnosed with an INHBE-associated disorder, e.g., metablic disorder, e.g., metabolic syndrome, a disorder of carbohydrates, e.g., type II diabetes, pre-diabetes, a lipid metabolism disorder, e.g., a hyperlipidemia, hypertension, a cardiovascular disease, a disorders of body weight) can be achieved in a number of different ways. For example, delivery may be performed by contacting a cell with a modulator of the invention either in vitro or in vivo. In vivo delivery may also be performed directly by administering a composition comprising a modulator to a subject. Alternatively, in vivo delivery may be performed indirectly by administering one or more vectors that encode and direct the expression of the modulator. These alternatives are discussed further below.

With respect to the oligonucleotide modulators of the invention, e.g., dsRNA agent or antisense polynucleotide agents, in general, any method of delivering a nucleic acid molecule (in vitro or in vivo) can be adapted for use with an iRNA of the invention (see e.g., Akhtar S. and Julian R L. (1992) Trends Cell. Biol. 2(5):139-144 and WO94/02595, which are incorporated herein by reference in their entireties). For in vivo delivery, factors to consider in order to deliver an iRNA molecule include, for example, biological stability of the delivered molecule, prevention of non-specific effects, and accumulation of the delivered molecule in the target tissue. RNA interference has also shown success with local delivery to the CNS by direct injection (Dorn, G., et al. (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya,Y., et al (2005) J. Neurophysiol. 93:594-602). Modification of the RNA or the pharmaceutical carrier can also permit targeting of the iRNA to the target tissue and avoid undesirable off-target effects. iRNA molecules can be modified by chemical conjugation to lipophilic groups such as cholesterol to enhance cellular uptake and prevent degradation. For example, an iRNA directed against ApoB conjugated to a lipophilic cholesterol moiety was injected systemically into mice and resulted in knockdown of apoB mRNA in both the liver and jejunum (Soutschek, J., et al (2004) Nature 432:173-178).

In an alternative embodiment, the modulator can be delivered using drug delivery systems such as a nanoparticle, a dendrimer, a polymer, liposomes, or a cationic delivery system. For example, positively charged cationic delivery systems facilitate binding of an iRNA molecule (negatively charged) and also enhance interactions at the negatively charged cell membrane to permit efficient uptake of an iRNA by the cell. Cationic lipids, dendrimers, or polymers can either be bound to an iRNA, or induced to form a vesicle or micelle (see e.g., Kim S H, et al (2008) Journal of Controlled Release 129(2):107-116) that encases an iRNA. The formation of vesicles or micelles further prevents degradation of the iRNA when administered systemically. Methods for making and administering cationic-iRNA complexes are well within the abilities of one skilled in the art (see e.g., Sorensen, D R, et al (2003) J. Mol. Biol 327:761-766; Verma, U N, et al (2003) Clin. Cancer Res. 9:1291-1300; Arnold, A S et al (2007) J. Hypertens. 25:197-205, which are incorporated herein by reference in their entirety). Some non-limiting examples of drug delivery systems useful for systemic delivery of iRNAs include DOTAP (Sorensen, D R., et al (2003), supra; Verma, U N, et al (2003), supra), “solid nucleic acid lipid particles” (Zimmermann, T S, et al (2006) Nature 441:111-114), cardiolipin (Chien, P Y, et al (2005) Cancer Gene Ther. 12:321-328; Pal, A, et al (2005) Int J. Oncol. 26:1087-1091), polyethyleneimine (Bonnet M E, et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A, et al (2007) Biochem. Soc. Trans. 35:61-67; Yoo, H., et al (1999) Pharm. Res. 16:1799-1804). In some embodiments, an iRNA forms a complex with cyclodextrin for systemic administration. Methods for administration and pharmaceutical compositions of iRNAs and cyclodextrins can be found in U.S. Pat. No. 7,427,605, which is herein incorporated by reference in its entirety. Certain aspects of the instant disclosure relate to a method of reducing the expression and/or acticity of INHBE in a cell, comprising contacting said cell with the modulator of the disclosure. In one embodiment, the cell is a hepatic cell, optionally a hepatocyte. In one embodiment, the cell is an extrahepatic cell.

### A. Vector Encoded Oligonucleotides of the Invention

Oligonucleotides targeting the INHBE gene can be expressed from transcription units inserted into DNA or RNA vectors (see, e.g., Couture, A, et al., TIG. (1996), 12:5-10; Skillern, A, et al., International PCT Publication No. WO 00/22113, Conrad, International PCT Publication No. WO 00/22114, and Conrad, U.S. Pat. No. 6,054,299). Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of an iRNA will generally require regulatory elements, e.g., promoters, enhancers, etc., to ensure the expression of the iRNA in target cells. Other aspects to consider for vectors and constructs are known in the art.

### V. Pharmaceutical Compositions of the Invention

The present invention also includes pharmaceutical compositions and formulations which include the modulators of the invention. In one embodiment, provided herein are pharmaceutical compositions containing a modulator, as described herein, and a pharmaceutically acceptable carrier. The pharmaceutical compositions containing the modulator are useful for preventing or treating an INHBE-associated disorder, e.g., metablic disorder, e.g., metabolic syndrome, a disorder of carbohydrates, e.g., type II diabetes, pre-diabetes, a lipid metabolism disorder, e.g., a hyperlipidemia, hypertension, a cardiovascular disease, a disorders of body weight.

Such pharmaceutical compositions are formulated based on the mode of delivery. One example is compositions that are formulated for systemic administration via parenteral delivery, e.g., by subcutaneous (SC), intramuscular (IM), or intravenous (IV) delivery. The pharmaceutical compositions of the invention may be administered in dosages sufficient to inhibit expression of an INHBE gene.

In some embodiments, the pharmaceutical compositions of the invention are sterile. In another embodiment, the pharmaceutical compositions of the invention are pyrogen free.

The pharmaceutical compositions of the invention may be administered in dosages sufficient to inhibit expression and/or actovoty of INHBE. In general, a suitable dose of a modulator of the invention will be in the range of about 0.001 to about 200.0 milligrams per kilogram body weight of the recipient per day, generally in the range of about 1 to 50 mg per kilogram body weight per day. Typically, a suitable dose of a modulator of the invention will be in the range of about 0.1 mg/kg to about 5.0 mg/kg, such as, about 0.3 mg/kg and about 3.0 mg/kg. A repeat-dose regimen may include administration of a therapeutic amount of a modulator on a regular basis, such as every month, once every 3-6 months, or once a year. In certain embodiments, the modulator is administered about once per month to about once per six months.

After an initial treatment regimen, the treatments can be administered on a less frequent basis. Duration of treatment can be determined based on the severity of disease.

In other embodiments, a single dose of the pharmaceutical compositions can be long lasting, such that doses are administered at not more than 1, 2, 3, or 4 month intervals. In some embodiments of the invention, a single dose of the pharmaceutical compositions of the invention is administered about once per month. In other embodiments of the invention, a single dose of the pharmaceutical compositions of the invention is administered quarterly (i.e., about every three months). In other embodiments of the invention, a single dose of the pharmaceutical compositions of the invention is administered twice per year (i.e., about once every six months).

The skilled artisan will appreciate that certain factors can influence the dosage and timing required to effectively treat a subject, including but not limited to mutations present in the subject, previous treatments, the general health or age of the subject, and other diseases present. Moreover, treatment of a subject with a prophylactically or therapeutically effective amount, as appropriate, of a composition can include a single treatment or a series of treatments.

The pharmaceutical compositions of the present disclosure can be administered in a number of ways depending upon whether local or systemic treatment is desired and upon the area to be treated. Administration may be topical (including ophthalmic, vaginal, rectal, intranasal, transdermal), oral, or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration.

The modulator can be delivered in a manner to target a particular tissue, such as the liver.

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the modulators featured in the disclosure are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). Modulators featured in the disclosure can be encapsulated within liposomes or can form complexes thereto, in particular to cationic liposomes. Alternatively, modulators can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-20 alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

In one embodiment, the modulators of the invention, are administered to a cell in a pharmaceutical composition by a topical route of administration. In one embodiment, the pharmaceutical composition may include a modulator mixed with a topical delivery agent. The topical delivery agent can be a plurality of microscopic vesicles. The microscopic vesicles can be liposomes. In some embodiments the liposomes are cationic liposomes.

In another embodiment, the modulator is admixed with a topical penetration enhancer. In one embodiment, the topical penetration enhancer is a fatty acid. The fatty acid can be arachidonic acid, oleic acid, lauric acid, caprylic acid, capric acid, myristic acid, palmitic acid, stearic acid, linoleic acid, linolenic acid, dicaprate, tricaprate, monolein, dilaurin, glyceryl 1-monocaprate, 1-dodecylazacycloheptan-2-one, an acylcarnitine, an acylcholine, or a C1-10 alkyl ester, monoglyceride, diglyceride or pharmaceutically acceptable salt thereof.

In another embodiment, the topical penetration enhancer is a bile salt. The bile salt can be cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24,25-dihydro-fusidate, sodium glycodihydrofusidate, polyoxyethylene-9-lauryl ether or a pharmaceutically acceptable salt thereof.

In another embodiment, the penetration enhancer is a chelating agent. The chelating agent can be EDTA, citric acid, a salicyclate, a N-acyl derivative of collagen, laureth-9, an N-amino acyl derivative of a beta-diketone or a mixture thereof.

In another embodiment, the penetration enhancer is a surfactant, e.g., an ionic or nonionic surfactant. The surfactant can be sodium lauryl sulfate, polyoxyethylene-9-lauryl ether, polyoxyethylene-20-cetyl ether, a perfluorchemical emulsion or mixture thereof.

In another embodiment, the penetration enhancer can be selected from a group consisting of unsaturated cyclic ureas, 1-alkyl-alkones, 1-alkenylazacyclo-alakanones, steroidal anti-inflammatory agents and mixtures thereof. In yet another embodiment the penetration enhancer can be a glycol, a pyrrol, an azone, or a terpenes.

In one aspect, the invention features a pharmaceutical composition including a modulator in an injectable dosage form. In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

The modulators of the invention can be incorporated into pharmaceutical compositions. Such compositions typically include one or more species of modulator and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration to a cell, e.g., a liver cell. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Pharmaceutical compositions of the present invention include, but are not limited to, solutions, emulsions, and liposome-containing formulations. These compositions can be generated from a variety of components that include, but are not limited to, preformed liquids, self-emulsifying solids, and self-emulsifying semisolids. Formulations include those that target the liver.

The pharmaceutical formulations of the present invention, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers.

### A. Additional Formulations

i. Emulsions

The compositions of the present invention can be prepared and formulated as emulsions.

Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, LV., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution either in the aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, LV., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, LV., Popovich N G., and Ansel H C., 2004, Lippincott Williams & Wilkins (8th ed.), New York, NY; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic, and amphoteric (see e.g., Ansel's Pharmaceutical Dosage Forms and Drug Delivery Systems, Allen, LV., Popovich N G., and Ansel H C., 2004, Lippincott Williams & Wilkins (8th ed.), New York, NY Rieger, in Pharmaceutical Dosage Forms, Lieberman, Rieger and Banker (Eds.), 1988, Marcel Dekker, Inc., New York, N.Y., volume 1, p. 285).

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives, and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

The application of emulsion formulations via dermatological, oral, and parenteral routes, and methods for their manufacture have been reviewed in the literature (see e.g., Ansel's Pharmaceutical Dosage Forms and Drug Delivery Systems, Allen, LV., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199).

ii. Microemulsions

In one embodiment of the present invention, the compositions of modulators are formulated as microemulsions. A microemulsion can be defined as a system of water, oil, and amphiphile which is a single optically isotropic and thermodynamically stable liquid solution (see e.g., Ansel's Pharmaceutical Dosage Forms and Drug Delivery Systems, Allen, LV., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215).

iii. Microparticles

An modulator of the invention may be incorporated into a particle, e.g., a microparticle. Microparticles can be produced by spray-drying, but may also be produced by other methods including lyophilization, evaporation, fluid bed drying, vacuum drying, or a combination of these techniques.

iv. Penetration Enhancers

In one embodiment, the present invention employs various penetration enhancers to effect the efficient delivery of modulators, e.g., nucleic acids, particularly iRNAs, to the skin of animals. Most drugs are present in solution in both ionized and nonionized forms. However, usually only lipid soluble or lipophilic drugs readily cross cell membranes. It has been discovered that even non-lipophilic drugs can cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers can be classified as belonging to one of five broad categories, i.e., surfactants, fatty acids, bile salts, chelating agents, and non-chelating non-surfactants (see e.g., Malmsten, M. Surfactants and polymers in drug delivery, Informa Health Care, New York, NY, 2002; Lee et al., Critical Reviews in Therapeutic Drug Carrier Systems, 1991, p. 92). Each of the above mentioned classes of penetration enhancers and their use in manufacture of pharmaceutical compositions and delivery of pharmaceutical agents are well known in the art.

v. Excipients

In contrast to a carrier compound, a “pharmaceutical carrier” or “excipient” is a pharmaceutically acceptable solvent, suspending agent, or any other pharmacologically inert vehicle for delivering one or more modulators to an animal. The excipient can be liquid or solid and is selected, with the planned manner of administration in mind, so as to provide for the desired bulk, consistency, etc., when combined with a nucleic acid and the other components of a given pharmaceutical composition. Such agent are well known in the art.

vi. Other Components

The compositions of the present invention can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings, or aromatic substances, and the like which do not deleteriously interact with the nucleic acid(s) of the formulation.

Aqueous suspensions can contain substances which increase the viscosity of the suspension including, for example, sodium carboxymethylcellulose, sorbitol, or dextran. The suspension can also contain stabilizers.

In some embodiments, pharmaceutical compositions featured in the invention include (a) one or more antisense polynucleotide agents and (b) one or more agents which function by a non-antisense inhibition mechanism and which are useful in treating an INHBE-associated disorder, e.g., a metabolic disorder.

Toxicity and prophylactic efficacy of such compounds can be determined by standard pharmaceutical procedures in cell cultures or experimental animals, e.g., for determining the LD50 (the dose lethal to 50% of the population) and the ED50 (the dose prophylactically effective in 50% of the population). The dose ratio between toxic and therapeutic effects is the therapeutic index and it can be expressed as the ratio LD50/ED50. Compounds that exhibit high therapeutic indices are preferred.

The data obtained from cell culture assays and animal studies can be used in formulating a range of dosage for use in humans. The dosage of compositions featured herein in the invention lies generally within a range of circulating concentrations that include the ED50, such as, an ED80 or ED90, with little or no toxicity. The dosage can vary within this range depending upon the dosage form employed and the route of administration utilized. For any compound used in the methods featured in the invention, the prophylactically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) or higher levels of inhibition as determined in cell culture. Such information can be used to more accurately determine useful doses in humans. Levels in plasma can be measured, for example, by high performance liquid chromatography.

In addition to their administration, as discussed above, the modulators featured in the invention can be administered in combination with other known agents used for the prevention or treatment of an INHBE-associated disorder, e.g., metabolic disorder. In any event, the administering physician can adjust the amount and timing of iRNA administration on the basis of results observed using standard measures of efficacy known in the art or described herein.

### VI. Methods For Inhibiting INHBE Expression and/or Activity

The present invention also provides methods of inhibiting expression and/or activity of INHBE in a cell. The methods include contacting a cell with a modulator, e.g., double stranded RNA agent, antisense polynucleotide agent, an antibody, a guideRNA effecting ADAR editing, or a guideRNA affecting CRISPR editing, in an amount effective to inhibit expression and/or activity of INHBE in the cell, thereby inhibiting expression and/or activity of INHBE in the cell. In some embodiments of the disclosure, expression of an INHBE gene is inhibited preferentially in the liver (e.g., hepatocytes).

Contacting of a cell with a modulator, may be done in vitro or in vivo. Contacting a cell in vivo with the modulator includes contacting a cell or group of cells within a subject, e.g., a human subject, with the modulator. Combinations of in vitro and in vivo methods of contacting a cell are also possible. Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc₃ ligand, or any other ligand that directs the modulator agent to a site of interest.

The term “inhibiting,” as used herein, is used interchangeably with “reducing,” “silencing,” “downregulating”, “suppressing”, and other similar terms, and includes any level of inhibition.

The phrase “inhibiting expression and/or activity of INHBE” is intended to refer to inhibition of expression of any INHBE (such as, e.g., a mouse INHBE gene, a rat INHBE gene, a monkey INHBE gene, or a human INHBE gene) as well as variants or mutants of an INHBE gene. Thus, the INHBE gene may be a wild-type INHBE gene, a mutant INHBE gene, or a transgenic INHBE gene in the context of a genetically manipulated cell, group of cells, or organism.

“Inhibiting expression and/or activity of INHBE” includes any level of inhibition of an INHBE gene, e.g., at least partial suppression of the expression and/or activity of INHBE. The expression and/or activity of INHBE may be assessed based on the level, or the change in the level, of any variable associated with INHBE gene expression, e.g., INHBE mRNA level or INHBE protein level. It is understood that INHBE is expressed predominantly in the liver.

The expression and/or activity of INHBE may also be assessed indirectly based on other variables associated with INHBE gene expression, e.g., level of inhibin subunit beta E expression in the cytoplasma, nuclear localization of inhibin subunit beta E, or expression of certain target genes or other genes under transcription control of inhibin subunit beta E.

Inhibition may be assessed by a decrease in an absolute or relative level of one or more variables that are associated with INHBE expression and/or activity compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In some embodiments of the methods of the invention, expression and/or activity of INHBE is inhibited by at least 50%, 55%, 60%, 65%, 70%, 75%, 80%, 85%, 90%, or 95%, or to below the level of detection of the assay. In some embodiments, expression and/or activity of INHBE is inhibited by at least 70%. It is further understood that inhibition of INHBE expression and/or activity in certain tissues, e.g., in liver, without a significant inhibition of expression in other tissues, e.g., brain, may be desirable.

In certain embodiments, inhibition of expression and/or activity in vivo is determined by knockdown of the human gene in a rodent expressing the human gene, e.g., an AAV-infected mouse expressing the human target gene (i.e., INHBE), e.g., when administered as a single dose, e.g., at 3 mg/kg at the nadir of RNA expression. Knockdown of expression of an endogenous gene in a model animal system can also be determined, e.g., after administration of a single dose at, e.g., 3 mg/kg at the nadir of RNA expression. Such systems are useful when the nucleic acid sequence of the human gene and the model animal gene are sufficiently close such that the human iRNA provides effective knockdown of the model animal gene. RNA expression in liver is determined using the PCR methods provided in Example 2.

Inhibition of the expression and/or activity of INHBE may be manifested by a reduction of the amount of mRNA expressed by a first cell or group of cells (such cells may be present, for example, in a sample derived from a subject) in which INHBE is transcribed and which has or have been treated (e.g., by contacting the cell or cells with a modulator of the invention, or by administering a modulator of the invention to a subject in which the cells are or were present) such that the expression of an INHBE gene is inhibited, as compared to a second cell or group of cells substantially identical to the first cell or group of cells but which has not or have not been so treated (control cell(s) not treated with a modulator or not treated with a modulator targeted to the gene of interest). In some embodiments, the inhibition is assessed by the method provided in Example 2 using a 10 nM siRNA concentration in the species matched cell line and expressing the level of mRNA in treated cells as a percentage of the level of mRNA in control cells, using the following formula:

$$\frac{( mRNA in control cells ) - ( mRNA in treated cells )}{( mRNA in control cells )} \cdot 100 \%$$

In other embodiments, inhibition of the expression and/or activity of INHBE may be assessed in terms of a reduction of a parameter that is functionally linked to INHBE gene expression, e.g., INHBE protein level in blood or serum from a subject. INHBE gene silencing may be determined in any cell expressing INHBE, either endogenous or heterologous from an expression construct, and by any assay known in the art.

Inhibition of the expression and/or activity of an INHBE protein may be manifested by a reduction in the level of the INHBE protein that is expressed by a cell or group of cells or in a subject sample (e.g., the level of protein in a blood sample derived from a subject). As explained above, for the assessment of mRNA suppression, the inhibition of protein expression levels in a treated cell or group of cells may similarly be expressed as a percentage of the level of protein in a control cell or group of cells, or the change in the level of protein in a subject sample, e.g., blood or serum derived therefrom.

A control cell, a group of cells, or subject sample that may be used to assess the inhibition of the expression and/or activity of INHBE includes a cell, group of cells, or subject sample that has not yet been contacted with a modulator agent of the invention. For example, the control cell, group of cells, or subject sample may be derived from an individual subject (e.g., a human or animal subject) prior to treatment of the subject with a modulator or an appropriately matched population control.

The level of INHBE mRNA that is expressed by a cell or group of cells may be determined using any method known in the art for assessing mRNA expression. In one embodiment, the level of expression of INHBE in a sample is determined by detecting a transcribed polynucleotide, or portion thereof, e.g., mRNA of the INHBE gene. RNA may be extracted from cells using RNA extraction techniques including, for example, using acid phenol/guanidine isothiocyanate extraction (RNAzol B; Biogenesis), RNeasy™ RNA preparation kits (Qiagen®) or PAXgene™ (PreAnalytix™, Switzerland). Typical assay formats utilizing ribonucleic acid hybridization include nuclear run-on assays, RT-PCR, RNase protection assays, northern blotting, in situ hybridization, and microarray analysis.

In some embodiments, the level of expression of INHBE is determined using a nucleic acid probe. The term “probe”, as used herein, refers to any molecule that is capable of selectively binding to a specific INHBE. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses and probe arrays. One method for the determination of mRNA levels involves contacting the isolated mRNA with a nucleic acid molecule (probe) that can hybridize to INHBE mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix® gene chip array. A skilled artisan can readily adapt known mRNA detection methods for use in determining the level of INHBE mRNA.

An alternative method for determining the level of expression of INHBE in a sample involves the process of nucleic acid amplification or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natd. Acad. Sci. USA 88:189-193), self sustained sequence replication (Guatelli et al. (1990) Proc. Natd. Acad. Sci. USA 87:1874-1878), transcriptional amplification system (Kwoh et al. (1989) Proc. Natd. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression of INHBE is determined by quantitative fluorogenic RT-PCR (i.e., the TaqMan™ System). In some embodiments, expression level is determined by the method provided in Example 2 using, e.g., a 10 nM siRNA concentration, in the species matched cell line.

The expression levels of INHBE mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of INHBE expression level may also comprise using nucleic acid probes in solution.

In some embodiments, the level of mRNA expression is assessed using branched DNA (bDNA) assays or real time PCR (qPCR). The use of these methods is described and exemplified in the Examples presented herein. In some embodiments, expression level is determined by the method provided in Example 2 using a 10 nM siRNA concentration in the species matched cell line.

The level of INHBE protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like.

In some embodiments, the efficacy of the methods of the invention are assessed by a decrease in INHBE mRNA or protein level (e.g., in a liver biopsy).

In some embodiments of the methods of the invention, the modulator is administered to a subject such that the modulator is delivered to a specific site within the subject. The inhibition of expression and/or activity of INHBE may be assessed using measurements of the level or change in the level of INHBE mRNA or INHBE protein in a sample derived from fluid or tissue from the specific site within the subject (e.g., liver or blood).

As used herein, the terms detecting or determining a level of an analyte are understood to mean performing the steps to determine if a material, e.g., protein, RNA, is present. As used herein, methods of detecting or determining include detection or determination of an analyte level that is below the level of detection for the method used.

### VII. Prophylactic and Treatment Methods of the Invention

The present invention also provides methods of using a modulator of the invention or a composition containing modulator of the invention to inhibit expression and/or activity of INHBE, thereby preventing or treating an INHBE-associated disorder, e.g., metabolic disorder, e.g., metabolic syndrome, a disorder of carbohydrates, e.g., type II diabetes, pre-diabetes, a lipid metabolism disorder, e.g., a hyperlipidemia, hypertension, a cardiovascular disease, a disorders of body weight. In the methods of the invention the cell may be contacted with the modulator in vitro or in vivo, i.e., the cell may be within a subject.

A cell suitable for treatment using the methods of the invention may be any cell that expresses an INHBE gene, e.g., a liver cell. A cell suitable for use in the methods of the invention may be a mammalian cell, e.g., a primate cell (such as a human cell, including human cell in a chimeric non-human animal, or a non-human primate cell, e.g., a monkey cell or a chimpanzee cell), or a non-primate cell. In certain embodiments, the cell is a human cell, e.g., a human liver cell. In the methods of the invention, INHBE expression is inhibited in the cell by at least 50, 55, 60, 65, 70, 75, 80, 85, 90, or 95, or to a level below the level of detection of the assay.

The in vivo methods of the invention may include administering to a subject a composition containing a modulator, such as an oligonucleotide, e.g., where the oligonucleotide includes a nucleotide sequence that is complementary to at least a part of an RNA transcript of the INHBE gene of the mammal to which the RNAi agent is to be administered. The composition can be administered by any means known in the art including, but not limited to oral, intraperitoneal, or parenteral routes, including intracranial (e.g., intraventricular, intraparenchymal, and intrathecal), intravenous, intramuscular, subcutaneous, transdermal, airway (aerosol), nasal, rectal, intraocular (e.g., periocular, conjunctival, subtenon, intracameral, intravitreal, intraocular, anterior or posterior juxtascleral, subretinal, subconjunctival, retrobulbar, or intracanalicular injection), intravenous, intramuscular, subcutaneous, transdermal, airway (aerosol), and topical (including buccal and sublingual) administration.

In certain embodiments, the compositions are administered by intravenous infusion or injection. In certain embodiments, the compositions are administered by subcutaneous injection. In certain embodiments, the compositions are administered by intramuscular injection.

The mode of administration may be chosen based upon whether local or systemic treatment is desired and based upon the area to be treated. The route and site of administration may be chosen to enhance targeting.

In one aspect, the present invention also provides methods for inhibiting the expression and/or activity of INHBE in a mammal. The methods include administering to the mammal a composition comprising a modulator, such as an oligonucleotide, e.g., an oligonucleotide that targets an INHBE gene, in a cell of the mammal and maintaining the mammal for a time sufficient to obtain degradation of the mRNA transcript of the INHBE gene, thereby inhibiting expression and/or activity of INHBE in the cell. Reduction in expression and/or activity can be assessed by any methods known in the art and by methods, e.g. qRT-PCR, described herein, e.g., in Example 2. Reduction in protein production can be assessed by any methods known it the art, e.g. ELISA. In certain embodiments, a puncture liver biopsy sample serves as the tissue material for monitoring the reduction in the INHBE gene or protein expression. In other embodiments, a blood sample serves as the subject sample for monitoring the reduction in the INHBE protein expression and/or actvity.

The present invention further provides methods of treatment in a subject in need thereof, e.g., a subject diagnosed with an INHBE-associated disorder, such as a metabolic disorder, e.g., metabolic syndrome, a disorder of carbohydrates, e.g., type II diabetes, pre-diabetes, a lipid metabolism disorder, e.g., a hyperlipidemia, hypertension, a cardiovascular disease, a disorders of body weight.

The present invention further provides methods of prophylaxis in a subject in need thereof. The treatment methods of the invention include administering a modulator of the invention to a subject, e.g., a subject that would benefit from a reduction of INHBE expression, in a prophylactically effective amount of a dsRNA targeting an INHBE gene or a pharmaceutical composition comprising a modulator of the invention.

In one aspect, the present invention provides methods of treating a subject having a disorder that would benefit from reduction in INHBE expression and/or activity, e.g., an INHBE-associated disorder, such as a metabolic disorder, e.g., diabetes.

Treatment of a subject that would benefit from a reduction and/or inhibition of INHBE expression and/or activity includes therapeutic treatment (e.g., a subject is having a metabolic disorder) and prophylactic treatment (e.g., the subject is not having a metablic disorder or a subject may be at risk of developing a metabolic disorder).

In some embodiments, the INHBE-associated disorder is a metabolic disorder. Examples of metablic disorder include but are not limited to, metabolic syndrome, a disorder of carbohydrates, e.g., type II diabetes, pre-diabetes, a lipid metabolism disorder, e.g., a hyperlipidemia, hypertension, a cardiovascular disease, a disorders of body weight.

In some embodiments, the INHBE-associated disorder is metabolic syndrome.

In some embodiments, the modulator is administered to a subject in an amount effective to inhibit INHBE expression and/or activity in a cell within the subject. The amount effective to inhibit INHBE expression and/or activity in a cell within a subject may be assessed using methods discussed above, including methods that involve assessment of the inhibition of INHBE mRNA, INHBE protein, or related variables, such as waist circunference.

With respect to oligonucleotide of the invention, an oligonucleotide of the invention may be administered as a “free oligonucleotide.” A free oligonucleotide is administered in the absence of a pharmaceutical composition. The naked oligonucleotide may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the oligonucleotide can be adjusted such that it is suitable for administering to a subject. Alternatively, an oligonucleotide of the invention may be administered as a pharmaceutical composition, such as an oligonucleotide liposomal formulation.

Subjects that would benefit from an inhibition of INHBE expression and/or activity are subjects susceptible to or diagnosed with an INHBE-associated disorder, such as a metablic disorder, e.g., metabolic syndrome, a disorder of carbohydrates, e.g., type II diabetes, pre-diabetes, a lipid metabolism disorder, e.g., a hyperlipidemia, hypertension, a cardiovascular disease, a disorders of body weight. In an embodiment, the method includes administering a composition featured herein such that expression and/or activity of INHBE is decreased, such as for about 1, 2, 3, 4, 5, 6, 1-6, 1-3, or 3-6 months per dose. In certain embodiments, the composition is administered once every 3-6 months.

In one embodiment, the modulators useful for the methods and compositions featured herein specifically target RNAs (primary or processed) of the target INHBE gene. Compositions and methods for inhibiting the expression of these genes using iRNAs can be prepared and performed as described herein.

Administration of the modulator according to the methods of the invention may result prevention or treatment of an INHBE-associated disorder, e.g., a metablic disorder, e.g., metabolic syndrome, a disorder of carbohydrates, e.g., type II diabetes, pre-diabetes, a lipid metabolism disorder, e.g., a hyperlipidemia, hypertension, a cardiovascular disease, a disorders of body weight. Subjects can be administered a therapeutic amount of modulator, such as about 0.01 mg/kg to about 200 mg/kg.

In one embodiment, the modulator is administered subcutaneously, i.e., by subcutaneous injection. One or more injections may be used to deliver the desired dose of modulator to a subject. The injections may be repeated over a period of time.

The administration may be repeated on a regular basis. In certain embodiments, after an initial treatment regimen, the treatments can be administered on a less frequent basis. A repeat-dose regimen may include administration of a therapeutic amount of modulator on a regular basis, such as once per month to once a year. In certain embodiments, the modulator is administered about once per month to about once every three months, or about once every three months to about once every six months.

The invention further provides methods and uses of a modulator or a pharmaceutical composition thereof for treating a subject that would benefit from reduction and/or inhibition of INHBE expression and/or activity, e.g., a subject having an INHBE-associated disorder, in combination with other pharmaceuticals and/or other therapeutic methods, e.g., with known pharmaceuticals and/or known therapeutic methods, such as, for example, those which are currently employed for treating these disorders.

Accordingly, in some aspects of the invention, the methods which include administration of a modulator of the invention, further include administering to the subject one or more additional therapeutic agents.

For example, in certain embodiments, a modulator of the invention is administered in combination with, e.g., an agent useful in treating an INHBE-associated disorder as described herein or otherwise known in the art. For example, additional agents and treatments suitable for treating a subject that would benefit from reducton in INHBE expression and/or activity, e.g., a subject having an INHBE-associated disorder, may include agents currently used to treat symptoms of INHBE-associated disorder.

Examples of the additional therapeutic agents which can be used with a modulator of the invention include, but are not limited to, insulin, a glucagon-like peptide 1 agonist (e.g., exenatide, liraglutide, dulaglutide, semaglutide, and pramlintide, a sulfonylurea (e.g., chlorpropamide, glipizide), a seglitinide (e.g., repaglinide, nateglinidie), biguanides (e.g., metformin), a thiazolidinedione, e.g, rosiglitazone, troglitazone, an alpha-glucosidase inhibitor (e.g., acarbose and meglitol), an SGLT2 inhibitor (e.g., dapagliflozin), a DPP-4 inhibitor (e.g., linagliptin), or an HMG-CoA reductase inhibitor, e.g., statins, such as atorvastatin (Lipitor), fluvastatin (Lescol), lovastatin (Mevacor), lovastatin extended-release (Altoprev), pitavastatin (Livalo), pravastatin (Pravachol), rosuvastatin (Crestor), and simvastatin (Zocor).

The modulator and an additional therapeutic agent and/or treatment may be administered at the same time and/or in the same combination, e.g., parenterally, or the additional therapeutic agent can be administered as part of a separate composition or at separate times and/or by another method known in the art or described herein.

### VIII. Kits

In certain aspects, the instant disclosure provides kits that include a suitable container containing a pharmaceutical formulation of a modulator of the invention.

Such kits include one or more modulator(s) and instructions for use, e.g., instructions for administering a prophylactically or therapeutically effective amount of modulator(s). The modulator may be in a vial or a pre-filled syringe. The kits may optionally further comprise means for administering the modulator (e.g., an injection device, such as a pre-filled syringe), or means for measuring the inhibition of INHBE (e.g., means for measuring the inhibition of INHBE mRNA, INHBE protein, and/or INHBE activity). Such means for measuring the inhibition of INHBE may comprise a means for obtaining a sample from a subject, such as, e.g., a plasma sample. The kits of the invention may optionally further comprise means for determining the therapeutically effective or prophylactically effective amount.

In certain embodiments the individual components of the pharmaceutical formulation may be provided in one container, e.g., a vial or a pre-filled syringe. Alternatively, it may be desirable to provide the components of the pharmaceutical formulation separately in two or more containers, e.g., one container for modulator preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

This invention is further illustrated by the following examples which should not be construed as limiting. The entire contents of all references, patents and published patent applications cited throughout this application, as well as the informal Sequence Listing and Figures, are hereby incorporated herein by reference.

### EXAMPLES

### Example 1. Identification of Association of INHBE Loss-of-Function with Waist-to-Hip Ratio in UK Biobank

Abdominal obesity is the most prevalent manifestation of metabolic syndrome (Després J. and Lemieux I. Nature 2006; 444:881-887) and is recognized as a contributor to cardiovascular disease and metabolic risk beyond body mass index (BMI) (Neeland I J et al. Lancet Diabetes & Endocrinology 2019; 7(9):715-725). Waist-to-hip ratio adjusted for BMI (WHRadjBMI) reflects abdominal adiposity and correlates with direct imaging of abdominal fat. Mendelian randomization studies have shown a causal relationship between WHRadjBMI and risk of type 2 diabetes and coronary heart disease along with ischemic stroke, glycemic traits and circulating lipids (Emdin C A et al. JAMA 2017; 317(6):626-634; Dale C E et al. Circulation 2017; 135(24):2373-2388).

Rare genetic variants were tested for association with waist-to-hip ratio adjusted for BMI using exome sequencing data from the UK Biobank (UKBB). UKBB, a large long-term biobank study in the United Kingdom (UK) is investigating the respective contributions of genetic predisposition and environmental exposure (including nutrition, lifestyle, medications etc.) to the development of disease (see, e.g., www.ukbiobank.ac.uk). The study is following about 500,000 volunteers in the UK, enrolled at ages from 40 to 69. Initial enrollment took place over four years from 2006, and the volunteers will be followed for at least 30 years thereafter. A plethora of phenotypic data has been collected including anthropometric measurements such as waist and hip circumference. Recently, the exome sequencing data (or the portion of the genomes composed of exons) from about 450,000 participants in the study has been obtained.

These whole exome sequences were used to identify rare predicted loss-of-function (pLOF) variants (i.e., frameshift, stop gain, splice donor or splice acceptor variants) called as high confidence by LOFTEE. WHR adjBMI were calculated for participants using manual measurements for waist circumference, hip circumference, and body mass index (BMI) which were taken at their UKBB assessment. WHR was calculated as the ratio of these two measurements. Using these data, along with age at recruitment and sex, a linear model was built modeling WHR (WHR˜Age+Sex+BMI). WHR adjBMI was defined using the residuals from this model.

Gene-based collapsing tests (i.e., burden tests) were used to look for associations between rare (minor allele frequency≤1%) pLOF variants and WHRadjBMI. Burden testing was performed in the unrelated White population (n=363,973) adjusting for age and genetic ancestry via 30 principal components. INHBE pLOF associated with a 0.22 standard deviation decrease in WHRadjBMI (Table A). INHBE was tested for association with additional quantitative traits and we detected associations with birth weight, WHR (not adjusted for BMI), triglycerides and HDL cholesterol (Table A). INHBE pLOF also has a lower odds ratio for hypertension, coronary heart disease and T2D (Table B)

The most common INHBE pLOF variant in the UKBB exome-sequencing data was a splice acceptor variant (rs150777893) carried by 536 out of 620 pLOF carriers. Tested as a single variant, rs150777893 significantly associated with decreased WHRadj BMI (Table C).

TABLE A
Association of INHBE pLOF with WHRadj BMI and other traits

| | | | Effect | N carrier |
|---|---|---|---|---|
| | Variant set | pvalue | (SD) | measured |
| WaistHipRatioAdjBMI | INHBE pLOF | 4.76E−08 | −0.22 | 618 |
| EarlyLife_Birth_weight | INHBE pLOF | 7.01E−07 | 0.26 | 345 |
| WaistHipRatio | INHBE pLOF | 3.45E−05 | −0.13 | 619 |
| Blood_Biochemistry_Triglycerides | INHBE pLOF | 0.001 | −0.13 | 594 |
| Blood_Biochemistry_HDL_cholesterol | INHBE pLOF | 0.01 | 0.10 | 550 |

TABLE B
Association of INHBE pLOF with hypertension, heart disease and T2D

| | | Odds ratio | n carrier | n |
|---|---|---|---|---|
| phenotype | pvalue | (95% CI) | cases | expected |
| I10_essential_primary_hypertension | 0.10 | 0.86 | 186 | 202.31 |
| | | (0.71, 1.03) | | |
| 125_chronic_ischaemic_heart_disease | 0.21 | 0.83 | 56 | 61.31 |
| | | (0.63, 1.11) | | |
| E11_non_insulin_dependent_diabetes | 0.36 | 0.87 | 45 | 50.30 |
| | | (0.64, 1.18) | | |

TABLE C
Association of splice acceptor variant rs150777893 with WHRadjBMI

| | | | | | | | | | | | N |
|---|---|---|---|---|---|---|---|---|---|---|---|
| | | | | | | Effect | | | | MAF | white |
| | chrom | pos | ref | alt | pvalue | (SD) | rsid | gene | consequence | white | carriers |
| WHRAdjBMI | 12 | 57456093 | G | C | 3.75E−08 | −0.24 | rs150777893 | INHBE | splice | 0.07% | 539 |
| | | | | | | | | | acceptor | | |
| | | | | | | | | | variant | | |

### Example 2. iRNA Synthesis

### Source of Reagents

Where the source of a reagent is not specifically given herein, such reagent can be obtained from any supplier of reagents for molecular biology at a quality/purity standard for application in molecular biology.

siRNA Design

siRNAs targeting the inhibin subunit beta E gene (INHBE, human: NCBI refseqID NM_031479.5, NCBI Gene ID: 83729) were designed using custom R and Python scripts. The human NM_031479.5 mRNA has a length of 2460 bases.

Detailed lists of the unmodified INHBE sense and antisense strand nucleotide sequences are shown in Table 2. Detailed lists of the modified INHBE sense and antisense strand nucleotide sequences are shown in Table 3.

It is to be understood that, throughout the application, a duplex name without a decimal is equivalent to a duplex name with a decimal which merely references the batch number of the duplex. For example, AD-959917 is equivalent to AD-959917.1.

siRNA Synthesis

siRNAs were designed, synthesized, and prepared using methods known in the art.

Briefly, siRNA sequences were synthesized on a 1 μmol scale using a Mermade 192 synthesizer (BioAutomation) with phosphoramidite chemistry on solid supports. The solid support was controlled pore glass (500-1000 Å) loaded with a custom GalNAc ligand (3′-GalNAc conjugates), universal solid support (AM Chemicals), or the first nucleotide of interest. Ancillary synthesis reagents and standard 2-cyanoethyl phosphoramidite monomers (2′-deoxy-2′-fluoro, 2′-O-methyl, RNA, DNA) were obtained from Thermo-Fisher (Milwaukee, WI), Hongene (China), or Chemgenes (Wilmington, MA, USA). Additional phosphoramidite monomers were procured from commercial suppliers, prepared in-house, or procured using custom synthesis from various CMOs. Phosphoramidites were prepared at a concentration of 100 mM in either acetonitrile or 9:1 acetonitrile:DMF and were coupled using 5-Ethylthio-1H-tetrazole (ETT, 0.25 M in acetonitrile) with a reaction time of 400 s. Phosphorothioate linkages were generated using a 100 mM solution of 3-((Dimethylamino-methylidene) amino)-3H-1,2,4-dithiazole-3-thione (DDTT, obtained from Chemgenes (Wilmington, MA, USA)) in anhydrous acetonitrile/pyridine (9:1 v/v). Oxidation time was 5 minutes. All sequences were synthesized with final removal of the DMT group (“DMT-Off”).

Upon completion of the solid phase synthesis, solid-supported oligoribonucleotides were treated with 300 μL of Methylamine (40% aqueous) at room temperature in 96 well plates for approximately 2 hours to afford cleavage from the solid support and subsequent removal of all additional base-labile protecting groups. For sequences containing any natural ribonucleotide linkages (2′-OH) protected with a tert-butyl dimethyl silyl (TBDMS) group, a second deprotection step was performed using TEA.3HF (triethylamine trihydrofluoride). To each oligonucleotide solution in aqueous methylamine was added 200 μL of dimethyl sulfoxide (DMSO) and 300 μL TEA.3HF and the solution was incubated for approximately 30 mins at 60° C. After incubation, the plate was allowed to come to room temperature and crude oligonucleotides were precipitated by the addition of 1 mL of 9:1 acetontrile:ethanol or 1:1 ethanol:isopropanol. The plates were then centrifuged at 4° C. for 45 mins and the supernatant carefully decanted with the aid of a multichannel pipette. The oligonucleotide pellet was resuspended in 20 mM NaOAc and subsequently desalted using a HiTrap size exclusion column (5 mL, GE Healthcare) on an Agilent L C system equipped with an autosampler, UV detector, conductivity meter, and fraction collector. Desalted samples were collected in 96 well plates and then analyzed by LC-MS and UV spectrometry to confirm identity and quantify the amount of material, respectively.

Duplexing of single strands was performed on a Tecan liquid handling robot. Sense and antisense single strands were combined in an equimolar ratio to a final concentration of 10 μM in 1× PBS in 96 well plates, the plate sealed, incubated at 100° C. for 10 minutes, and subsequently allowed to return slowly to room temperature over a period of 2-3 hours. The concentration and identity of each duplex was confirmed and then subsequently utilized for in vitro screening assays.

### Example 3. In Vitro siRNA Screening Methods

### Cell Culture and 384-Well Transfections

Hep3b cells (ATCC, Manassas, VA) are grown to near confluence at 37° C. in an atmosphere of 5% CO₂ in Eagle's Minimum Essential Medium (Gibco) supplemented with 10% FBS (ATCC) before being released from the plate by trypsinization. Transfection is carried out by adding 7.5 I of Opti-MEM plus 0.1 μl of Lipofectamine RNAiMax per well (Invitrogen, Carlsbad CA. cat #13778-150) to 2.5 μl of each siRNA duplex to an individual well in a 384-well plate. The mixture is then incubated at room temperature for 15 minutes. Forty l of complete growth media without antibiotic containing ˜1.5×10′ cells are then added to the siRNA mixture. Cells are incubated for 24 hours prior to RNA purification. Single dose experiments are performed at 10 nM, 1 nM, and 0.1 nM final duplex concentration.

Total RNA isolation using DYNABEADS mRNA Isolation Kit (Invitrogen™, part #: 610-12)

Cells are lysed in 75 μl of Lysis/Binding Buffer containing 3 μL of beads per well and mixed for 10 minutes on an electrostatic shaker. The washing steps are automated on a Biotek EL406, using a magnetic plate support. Beads are washed (in 90 L) once in Buffer A, once in Buffer B, and twice in Buffer E, with aspiration steps in between. Following a final aspiration, complete 10 μL RT mixture is added to each well, as described below.

cDNA Synthesis Using ABI High Capacity cDNA Reverse Transcription Kit (Applied Biosystems, Foster City, CA, Cat #4368813)

A master mix of 1 μl 10× Buffer, 0.4 μl 25×dNTPs, 1 μl Random primers, 0.5 μl Reverse Transcriptase, 0.5 μl RNase inhibitor and 6.6 μl of H₂O per reaction are added per well. Plates are sealed, agitated for 10 minutes on an electrostatic shaker, and then incubated at 37 degrees C. for 2 hours. Following this, the plates are agitated at 80 degrees C. for 8 minutes.

### Real Time PCR

Two microlitre (μl) of cDNA are added to a master mix containing 0.5 μl of human GAPDH TaqMan Probe (4326317E), 0.5 μl human INHBE, 2 μl nuclease-free water and 5 μl Lightcycler 480 probe master mix (Roche Cat #04887301001) per well in a 384 well plates (Roche cat #04887301001). Real time PCR is done in a LightCycler480 Real Time PCR system (Roche).

To calculate relative fold change, data are analyzed using the AACt method and normalized to assays performed with cells transfected with 10 nM AD-1955, or mock transfected cells. IC₅₀s are calculated using a 4 parameter fit model using XLFit and normalized to cells transfected with AD-1955 or mock-transfected. The sense and antisense sequences of AD-1955 are: sense: cuuAcGcuGAGuAcuucGAdTsdT and antisense UCGAAGuACUcAGCGuAAGdTsdT.

### Example 4. Antisense Polynucleotide Agent Synthesis

Bioinformatics A set of antisense polynucleotide agents targeting the inhibin subunit beta E gene (INHBE, human: NCBI refseqID NM_031479.5, NCBI Gene ID: 83729) were designed and synthesized using standard synthesis methods well known in the art.

A detailed list of the unmodified nucleotide sequences of antisense molecules targeting INHBE is shown in Table 4 and a detailed list of the modified nucleotide sequences of antisense molecules targeting INHBE is shown in Table 5.

### Example 5. In Vitro Fluorescence-Based Branched DNA (bDNA) Screening Assay

### Summary

A panel of 32 human cell lines was screened for expression of INHBE by measuring the level of INHBE transcript using a fluorescence-based branched DNA (bDNA) assay. A cell line (Hep3B) with sufficient expression (at least 50-fold above background relative luminescence units (RLUs)) was identified as an in vitro model to screen INHBE siRNAs and ASOs for target knockdown.

### Materials and Methods

Quantigene probesets were designed for human INHBE and human GapDH mRNA (housekeeper gene for normalization) and to Ahsal, which was used as a positive control in screens. Probeset oligos were ordered from ThermoFisher Scientific and synthesized by Metabion. Probeset sequences, without their proprietary parts, for INHBE, GapdDH and Ahsal.

To identify a cell line with sufficient target expression for screening (RLUs at least ˜50-fold above background), lysates from 32 human cell lines were tested for INHBE target expression by Quantigene Singleplex Assay. Of each cell line, two different volumes of lysate were analyzed with a concentration of 200,000 cells/mL lysate. The Quantigene assay was performed according to the manufacturer's protocol for Quantigene Singleplex. Luminescence was read using 1420 Luminescence Counter (WALLAC VICTOR Light, Perkin Elmer, Rodgau-Jugesheim, Germany) following 30 minutes incubation at RT in the dark.

### In Vitro Screen & Dose Response in Hep3B Cells

Hep3b cells (ATCC, Manassas, VA) are grown to near confluence at 37° C. in an atmosphere of 5% CO₂ in Eagle's Minimum Essential Medium (Gibco) supplemented with 10% FBS (ATCC) before being released from the plate by trypsinization.

For transfection of Hep3B cells, cells were seeded at a density of 15,000 cells/well into 96-well tissue culture plates (#655180, GBO, Germany). Transfection of siRNAs was carried out with Lipofectamine RNAiMax, antisense oligonucleotides (ASOs) were transfected with Lipofectamine 2000 (both from Invitrogen/Life Technologies, Karlsruhe, Germany) according to manufacturer's instructions for reverse transfection. The in vitro screen was performed with siRNAs/ASOs in quadruplicates at 10 nM and 1 nM, with siRNAs targeting Ahsal, Firefly-Luciferase and Renilla-Luciferase as unspecific controls and a mock transfection. After 24 hours of incubation with siRNAs/ASOs, medium was removed and cells were lysed in 150 μl Medium-Lysis Mixture (1 volume lysis buffer, 2 volumes cell culture medium) and then incubated at 53° C. for 30 minutes. bDNA assay was performed according to manufacturer's instructions with a probeset directed to human INHBE. Luminescence was read using 1420 Luminescence Counter (WALLAC VICTOR Light, Perkin Elmer, Rodgau-Jugesheim, Germany) following 30 minutes incubation at RT in the dark.

The Ahsa1 siRNA served as an unspecific control for INHBE target mRNA expression and as positive control for transfection efficiency with regards to Ahsa1 mRNA level. By hybridization with an Ahsa1 probeset, mock-transfection served as controls for Ahsa1 mRNA level. Transfection efficiency for each 96-well plate and both doses in the in vitro dose screen was calculated by relating the Ahsa1-level in cells treated with Ahsa1 siRNA/ASO (normalized to GapDH) to Ahsa1 levels in mock-treated cells.

For each well, the target mRNA level was normalized to the respective GapDH mRNA level. The activity of a given siRNA/ASO was expressed as percent of mRNA concentration of the respective target (normalized to GapDH mRNA) in treated cells, relative to the target mRNA concentration (normalized to GapDH mRNA) averaged across control wells or mock transfected wells (DRCs). The half maximal inhibitory concentration (IC₅₀) was determined with XLfit software (an Excel add-in) using a 4 parameter logistic model with the formula

$$Y = bottom + ( top - bottom ) / ( 1 + {( \frac{I C 50}{X} )}^{Hillslope} )$$

and with Top constrained to a value of 100%. IC₈₀ (80% inhibitory concentration) was calculated from IC₅₀ using the formula

$$I {C}_{F} = {( \frac{F}{100 - F} )}^{\frac{1}{H}} \times I C 50 ,$$

where F is the percentage inhibition and H is the hill slope.

Table 6 shows the results of in vitro screen in cells transfected with the indicated antisense molecules.

TABLE 1
Abbreviations of nucleotide monomers used in nucleic acid sequence representation. It will be understood that these monomers,
when present in an oligonucleotide, are mutually linked by 5′-3′-phosphodiester bonds; and it is understood that when the
nucleotide contains a 2′-fluoro modification, then the fluoro replaces the hydroxy at that position in the parent nucleotide (i.e.,
it is a 2′-deoxy-2′-fluoronucleotide). It is to be further understood that the nucleotide abbreviations in the table omit the 3′-
phosphate (i.e., they are 3′-OH) when placed at the 3′-terminal position of an oligonucleotide.

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O—methyladenosine-3′-phosphate |
| as | 2′-O—methyladenosine-3′-phosphorothioate |
| C | 2′-O—methylcytidine-3′-phosphate |
| CS | 2′-O—methylcytidine-3′-phosphorothioate |
| g | 2′-O—methylguanosine-3′-phosphate |
| gs | 2′-O—methylguanosine-3′-phosphorothioate |
| t | 2′-O—methyl-5-methyluridine-3′-phosphate |
| ts | 2′-O—methyl-5-methyluridine-3′-phosphorothioate |
| u | 2′-O—methyluridine-3′-phosphate |
| us | 2′-O—methyluridine-3′-phosphorothioate |
| S | phosphorothioate linkage |
| L10 | N-(cholesterylcarboxamidocaproyl)-4-hydroxyprolinol (Hyp-C6-Chol) |
| | 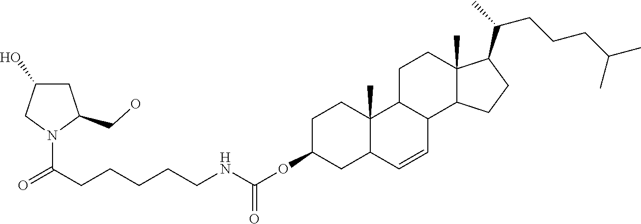 [SMILES: CC(C)CCC[C@@H](C)[C@H]1CCC2C3C=CC4C[C@@H](OC(=O)NCCCCCC(=O)N5C[C@H](O)C[C@H]5CO)CC[C@]4(C)C3CC[C@@]21C] |
| L96 | N-[tris(GalNAc-alkyl)-amidodecanoyl)]-4-hydroxyprolinol |
| | (Hyp-(GalNAc-alkyl)3) |
| | (2S,4R)-1-[29-[2-(acetylamino)-2-deoxy-β-D-galactopyranosyl]oxy]-14,14-bis[[3-[[3-[[5-[2-(acetylamino)-2- |
| | deoxy-β-D-galactopyranosyl]oxy]-1-oxopentyl]amino]propyl]amino]-3-oxopropoxy]methyl]-1,12,19,25- |
| | tetraoxo-16-oxa-13,20,24-triazanonacos-1-yl]-4-hydroxy-2-hydroxymethylpyrrolidine |
| | 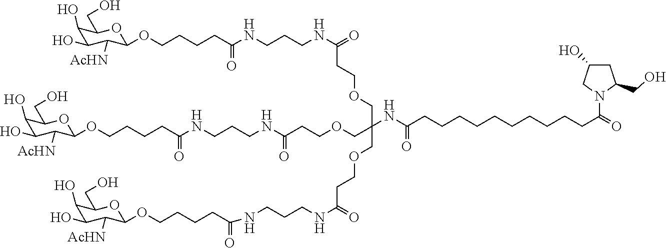 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O] |
| uL96 | 2′-O—methyluridine-3′-phosphate ((2S,4R)-1-[29-[[2-(acetylamino)-2-deoxy-β-D-galactopyranosyl] |
| | oxy]-14,14-bis[[3-[[3-[[5-[[2-(acetylamino)-2-deoxy-β-D-galactopyranosyl]oxy]-1-oxopentyl]amino] |
| | propyl]amino]-3-oxopropoxy]methyl]-1,12,19,25-tetraoxo-16-oxa-13,20,24-triazanonacos-1-yl]-4- |
| | hydroxy-2-pyrrolidinyl)methyl ester |
| | 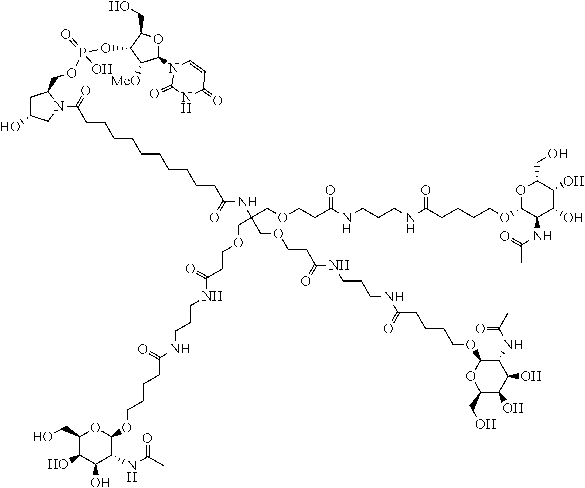 [SMILES: CO[C@@H]1[C@H](OP(=O)(O)OC[C@@H]2C[C@@H](O)CN2C(=O)CCCCCCCCCCC(=O)NC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)COCCC(=O)NCCCNC(=O)CCCCO[C@@H]2O[C@H](CO)[C@H](O)[C@H](O)[C@H]2NC(C)=O)[C@@H](CO)O[C@H]1n1ccc(=O)[nH]c1=O] |
| Y34 | 2-hydroxymethyl-tetrahydrofurane-4-methoxy-3-phosphate (abasic 2′-OMe furanose) |
| | 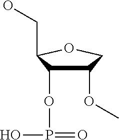 |
| Y44 | inverted abasic DNA (2-hydroxymethyl-tetrahydrofurane-5-phosphate) |
| | 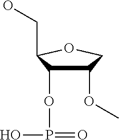 |
| (Agn) | Adenosine-glycol nucleic acid (GNA) |
| (Cgn) | Cytidine-glycol nucleic acid (GNA) |
| (Ggn) | Guanosine-glycol nucleic acid (GNA) |
| (Tgn) | Thymidine-glycol nucleic acid (GNA) S-Isomer |
| P | Phosphate |
| VP | Vinyl-phosphonate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythimidine-3′-phosphate |
| dTs | 2′-deoxythimidine-3′-phosphorothioate |
| dU | 2′-deoxyuridine |
| dUs | 2′-deoxyuridine-3′-phosphorothioate |
| (C2p) | cytidine-2′-phosphate |
| (G2p) | guanosine-2′-phosphate |
| (U2p) | uridine-2′-phosphate |
| (A2p) | adenosine-2′-phosphate |
| (Ahd) | 2′-O—hexadecyl-adenosine-3′-phosphate |
| (Ahds) | 2′-O—hexadecyl-adenosine-3′-phosphorothioate |
| (Chd) | 2′-O—hexadecyl-cytidine-3′-phosphate |
| (Chds) | 2′-O—hexadecyl-cytidine-3′-phosphorothioate |
| (Ghd) | 2′-O—hexadecyl-guanosine-3′-phosphate |
| (Ghds) | 2′-O—hexadecyl-guanosine-3′-phosphorothioate |
| (Uhd) | 2′-O—hexadecyl-uridine-3′-phosphate |
| (Uhds) | 2′-O—hexadecyl-uridine-3′-phosphorothioate |
| (Aeo) | 2′-O—methoxyethyladenosine-3′-phosphate |
| (Aeos) | 2′-O—methoxyethyladenosine-3′-phosphorothioate |
| (Geo) | 2′-O—methoxyethylguanosine-3′-phosphate |
| (Geos) | 2′-O—methoxyethylguanosine-3′-phosphorothioate |
| (Teo) | 2′-O—methoxyethyl-5-methyluridine-3′-phosphate |
| (Teos) | 2′-O—methoxyethyl-5-methyluridine-3′-phosphorothioate |
| (m5Ceo) | 2′-O—methoxyethyl-5-methylcytidine-3′-phosphate |
| (m5Ceos) | 2′-O—methoxyethyl-5-methylcytidine-3′-phosphorothioate |
| (m5dC) | 5′-methyl-deoxycytidine-3′-phosphate |
| (m5dCs) | 5′-methyl-deoxycytidine-3′-phosphorothioate |

TABLE 2
Unmodified Sense and Antisense Strand Sequences of INHBE dsRNA Agents

| | | | Range in | | |
|---|---|---|---|---|---|
| | | SEQ | INHBE | | SEQ |
| | Sense Sequence | ID | mRNA | Antisense Sequence | ID |
| Duplex Name | 5′ to 3′ | NO: | NM_031479.5 | 5′ to 3′ | NO: |
| AD-1656008 | UAGCCAGACAUGAGCUGUGAU | | 1-23 | AUCACAGCUCAUGUCUGGCUACU | |
| AD-1656026 | GAGGGUCAAGCACAGCUAUCU | | 19-41 | AGAUAGCUGUGCUUGACCCUCAC | |
| AD-1656043 | AUCCAUCAGAUGAUCUACUUU | | 36-58 | AAAGUAGAUCAUCUGAUGGAUAG | |
| AD-1656054 | GAUCUACUUUCAGCCUUCCUU | | 47-69 | AAGGAAGGCUGAAAGUAGAUCAU | |
| AD-1656074 | GAGUCCCAGACAAUAGAAGAU | | 67-89 | AUCUUCUAUUGUCUGGGACUCAG | |
| AD-1656086 | AUAGAAGACAGGUGGCUGUAU | | 79-101 | AUACAGCCACCUGUCUUCUAUUG | |
| AD-1656097 | GUGGCUGUACCCUUGGCCAAU | | 90-112 | AUUGGCCAAGGGUACAGCCACCU | |
| AD-1656108 | CUUGGCCAAGGGUAGGUGUGU | | 101-123 | ACACACCUACCCUUGGCCAAGGG | |
| AD-1656125 | GUGGCAGUGGUGUCUGCUGUU | | 118-140 | AACAGCAGACACCACUGCCACAC | |
| AD-1656139 | UGCUGUCACUGUGCCCUCAUU | | 132-154 | AAUGAGGGCACAGUGACAGCAGA | |
| AD-1656146 | AGCAAUCAGACUCAACAGACU | | 160-182 | AGUCUGUUGAGUCUGAUUGCUGG | |
| AD-1656164 | ACGGAGCAACUGCCAUCCGAU | | 178-200 | AUCGGAUGGCAGUUGCUCCGUCU | |
| AD-1656185 | GCUCCUGAACCAGGGCCAUUU | | 199-221 | AAAUGGCCCUGGUUCAGGAGCCU | |
| AD-1656196 | AGGGCCAUUCACCAGGAGCAU | | 210-232 | AUGCUCCUGGUGAAUGGCCCUGG | |
| AD-1656220 | GCUCCCUGAUGUCCAGCUCUU | | 234-256 | AAGAGCUGGACAUCAGGGAGCCG | |
| AD-1656233 | CAGCUCUGGCUGGUGCUGCUU | | 247-269 | AAGCAGCACCAGCCAGAGCUGGA | |
| AD-1656254 | UGGGCACUGGUGCGAGCACAU | | 268-290 | AUGUGCUCGCACCAGUGCCCACA | |
| AD-1656260 | AGGGUCUGUGUGUCCCUCCUU | | 294-316 | AAGGAGGGACACACAGACCCUGU | |
| AD-1656265 | CAAGCAGAACGAGCUCUGGUU | | 337-359 | AACCAGAGCUCGUUCUGCUUGGG | |
| AD-1656280 | CUGGUGCUGGAGCUAGCCAAU | | 352-374 | AUUGGCUAGCUCCAGCACCAGAG | |
| AD-1656292 | CUAGCCAAGCAGCAAAUCCUU | | 364-386 | AAGGAUUUGCUGCUUGGCUAGCU | |
| AD-1656307 | AUCCUGGAUGGGUUGCACCUU | | 379-401 | AAGGUGCAACCCAUCCAGGAUUU | |
| AD-1656319 | UUGCACCUGACCAGUCGUCCU | | 391-413 | AGGACGACUGGUCAGGUGCAACC | |
| AD-1656333 | UCGUCCCAGAAUAACUCAUCU | | 405-427 | AGAUGAGUUAUUCUGGGACGACU | |
| AD-1656360 | CCCUCCGGAGACUACAGCCAU | | 452-474 | AUGGCUGUAGUCUCCGGAGGGCU | |
| AD-1656372 | UACAGCCAGGGAGUGUGGCUU | | 464-486 | AAGCCACACUCCCUGGCUGUAGU | |
| AD-1656383 | AGUGUGGCUCCAGGGAAUGGU | | 475-497 | ACCAUUCCCUGGAGCCACACUCC | |
| AD-1656392 | CAUCAGCUUUGCUACUGUCAU | | 504-526 | AUGACAGUAGCAAAGCUGAUGAC | |
| AD-1656406 | CUGUCACAGACUCCACUUCAU | | 518-540 | AUGAAGUGGAGUCUGUGACAGUA | |
| AD-1656417 | UCCACUUCAGCCUACAGCUCU | | 529-551 | AGAGCUGUAGGCUGAAGUGGAGU | |
| AD-1656437 | CCUGCUCACUUUUCACCUGUU | | 549-571 | AACAGGUGAAAAGUGAGCAGGGA | |
| AD-1656449 | UCACCUGUCCACUCCUCGGUU | | 561-583 | AACCGAGGAGUGGACAGGUGAAA | |
| AD-1656468 | UCCCACCACCUGUACCAUGCU | | 580-602 | AGCAUGGUACAGGUGGUGGGACC | |
| AD-1656480 | UACCAUGCCCGCCUGUGGCUU | | 592-614 | AAGCCACAGGCGGGCAUGGUACA | |
| AD-1656492 | CCCUUCCUGGCACUCUUUGCU | | 626-648 | AGCAAAGAGUGCCAGGAAGGGUG | |
| AD-1656504 | CUCUUUGCUUGAGGAUCUUCU | | 638-660 | AGAAGAUCCUCAAGCAAAGAGUG | |
| AD-1656535 | ACUCUCCUGGCUGAGCACCAU | | 694-716 | AUGGUGCUCAGCCAGGAGAGUGC | |
| AD-1656547 | GAGCACCACAUCACCAACCUU | | 706-728 | AAGGUUGGUGAUGUGGUGCUCAG | |
| AD-1656559 | ACCAACCUGGGCUGGCAUACU | | 718-740 | AGUAUGCCAGCCCAGGUUGGUGA | |
| AD-1656570 | CUGGCAUACCUUAACUCUGCU | | 729-751 | AGCAGAGUUAAGGUAUGCCAGCC | |
| AD-1656587 | UGCCCUCUAGUGGCUUGAGGU | | 746-768 | ACCUCAAGCCACUAGAGGGCAGA | |
| AD-1656591 | AGAAGUCUGGUGUCCUGAAAU | | 770-792 | AUUUCAGGACACCAGACUUCUCA | |
| AD-1656602 | GUCCUGAAACUGCAACUAGAU | | 781-803 | AUCUAGUUGCAGUUUCAGGACAC | |
| AD-1656622 | ACAGCACAGUUACUGGACAAU | | 821-843 | AUUGUCCAGUAACUGUGCUGUUG | |
| AD-1656634 | CUGGACAACCGAGGCGGCUCU | | 833-855 | AGAGCCGCCUCGGUUGUCCAGUA | |
| AD-1656647 | GCGGCUCUUGGACACAGCAGU | | 846-868 | ACUGCUGUGUCCAAGAGCCGCCU | |
| AD-1656667 | GACACCAGCAGCCCUUCCUAU | | 866-888 | AUAGGAAGGGCUGCUGGUGUCCU | |
| AD-1656679 | CCUUCCUAGAGCUUAAGAUCU | | 878-900 | AGAUCUUAAGCUCUAGGAAGGGC | |
| AD-1656690 | CUUAAGAUCCGAGCCAAUGAU | | 889-911 | AUCAUUGGCUCGGAUCUUAAGCU | |
| AD-1656701 | AGCCAAUGAGCCUGGAGCAGU | | 900-922 | ACUGCUCCAGGCUCAUUGGCUCG | |
| AD-1656716 | AGGCGAGACCAUUACGUAGAU | | 973-995 | AUCUACGUAAUGGUCUCGCCUGC | |
| AD-1656728 | UACGUAGACUUCCAGGAACUU | | 985-1007 | AAGUUCCUGGAAGUCUACGUAAU | |
| AD-1656740 | CAGGAACUGGGAUGGCGGGAU | | 997-1019 | AUCCCGCCAUCCCAGUUCCUGGA | |
| AD-1656754 | GCGGGACUGGAUACUGCAGCU | | 1011-1033 | AGCUGCAGUAUCCAGUCCCGCCA | |
| AD-1656762 | UACCAGCUGAAUUACUGCAGU | | 1039-1061 | ACUGCAGUAAUUCAGCUGGUACC | |
| AD-1656775 | ACUGCAGUGGGCAGUGCCCUU | | 1052-1074 | AAGGGCACUGCCCACUGCAGUAA | |
| AD-1656792 | CAGGCAUUGCUGCCUCUUUCU | | 1091-1113 | AGAAAGAGGCAGCAAUGCCUGGG | |
| AD-1656808 | UUUCCAUUCUGCCGUCUUCAU | | 1107-1129 | AUGAAGACGGCAGAAUGGAAAGA | |
| AD-1656820 | CGUCUUCAGCCUCCUCAAAGU | | 1119-1141 | ACUUUGAGGAGGCUGAAGACGGC | |
| AD-1656832 | CCUCAAAGCCAACAAUCCUUU | | 1131-1153 | AAAGGAUUGUUGGCUUUGAGGAG | |
| AD-1656849 | CUUGGCCUGCCAGUACCUCCU | | 1148-1170 | AGGAGGUACUGGCAGGCCAAGGA | |
| AD-1656862 | UACCUCCUGUUGUGUCCCUAU | | 1161-1183 | AUAGGGACACAACAGGAGGUACU | |
| AD-1656873 | GUGUCCCUACUGCCCGAAGGU | | 1172-1194 | ACCUUCGGGCAGUAGGGACACAA | |
| AD-1656876 | CUCUCUCUCCUCUACCUGGAU | | 1195-1217 | AUCCAGGUAGAGGAGAGAGAGGG | |
| AD-1656888 | UACCUGGAUCAUAAUGGCAAU | | 1207-1229 | AUUGCCAUUAUGAUCCAGGUAGA | |
| AD-1656900 | AAUGGCAAUGUGGUCAAGACU | | 1219-1241 | AGUCUUGACCACAUUGCCAUUAU | |
| AD-1656915 | AAGACGGAUGUGCCAGAUAUU | | 1234-1256 | AAUAUCUGGCACAUCCGUCUUGA | |
| AD-1656926 | GCCAGAUAUGGUGGUGGAGGU | | 1245-1267 | ACCUCCACCACCAUAUCUGGCAC | |
| AD-1656942 | GAGGCCUGUGGCUGCAGCUAU | | 1261-1283 | AUAGCUGCAGCCACAGGCCUCCA | |
| AD-1656954 | UGCAGCUAGCAAGAGGACCUU | | 1273-1295 | AAGGUCCUCUUGCUAGCUGCAGC | |
| AD-1656958 | CUUUGGAGUGAAGAGACCAAU | | 1297-1319 | AUUGGUCUCUUCACUCCAAAGCC | |
| AD-1656969 | AGAGACCAAGAUGAAGUUUCU | | 1308-1330 | AGAAACUUCAUCUUGGUCUCUUC | |
| AD-1656996 | CAGGGCAUCUGUGACUGGAGU | | 1335-1357 | ACUCCAGUCACAGAUGCCCUGUG | |
| AD-1657013 | GAGGCAUCAGAUUCCUGAUCU | | 1352-1374 | AGAUCAGGAAUCUGAUGCCUCCA | |
| AD-1657022 | ACCCAACAACCACCUGGCAAU | | 1381-1403 | AUUGCCAGGUGGUUGUUGGGUUG | |
| AD-1657037 | GGCAAUAUGACUCACUUGACU | | 1396-1418 | AGUCAAGUGAGUCAUAUUGCCAG | |
| AD-1657045 | GACCCAAAUGGGCACUUUCUU | | 1424-1446 | AAGAAAGUGCCCAUUUGGGUCCC | |
| AD-1657058 | ACUUUCUUGUCUGAGACUCUU | | 1437-1459 | AAGAGUCUCAGACAAGAAAGUGC | |
| AD-1657069 | UGAGACUCUGGCUUAUUCCAU | | 1448-1470 | AUGGAAUAAGCCAGAGUCUCAGA | |
| AD-1657085 | UCCAGGUUGGCUGAUGUGUUU | | 1464-1486 | AAACACAUCAGCCAACCUGGAAU | |
| AD-1657099 | UGUGUUGGGAGAUGGGUAAAU | | 1478-1500 | AUUUACCCAUCUCCCAACACAUC | |
| AD-1657113 | GGUAAAGCGUUUCUUCUAAAU | | 1492-1514 | AUUUAGAAGAAACGCUUUACCCA | |
| AD-1657119 | UACCCAGAAAGCAUGAUUUCU | | 1518-1540 | AGAAAUCAUGCUUUCUGGGUAGA | |
| AD-1657133 | GAUUUCCUGCCCUAAGUCCUU | | 1532-1554 | AAGGACUUAGGGCAGGAAAUCAU | |
| AD-1657147 | AGUCCUGUGAGAAGAUGUCAU | | 1546-1568 | AUGACAUCUUCUCACAGGACUUA | |
| AD-1657164 | UCAGGGACUAGGGAGGGAGGU | | 1563-1585 | ACCUCCCUCCCUAGUCCCUGACA | |
| AD-1657185 | AAUUACUUAGCCUCUCCCAAU | | 1600-1622 | AUUGGGAGAGGCUAAGUAAUUUU | |
| AD-1657202 | CAAGAUGAGAAAGUCCUCAAU | | 1617-1639 | AUUGAGGACUUUCUCAUCUUGGG | |
| AD-1657211 | GGAGGAAGCAGAUAGAUGGUU | | 1646-1668 | AACCAUCUAUCUGCUUCCUCCUC | |
| AD-1657234 | GCAGGCUUGAAGCAGGGUAAU | | 1669-1691 | AUUACCCUGCUUCAAGCCUGCUG | |
| AD-1657245 | GCAGGGUAAGCAGGCUGGCCU | | 1680-1702 | AGGCCAGCCUGCUUACCCUGCUU | |
| AD-1657261 | GGCCCAGGGUAAGGGCUGUUU | | 1696-1718 | AAACAGCCCUUACCCUGGGCCAG | |
| AD-1657274 | GGCUGUUGAGGUACCUUAAGU | | 1709-1731 | ACUUAAGGUACCUCAACAGCCCU | |
| AD-1657286 | ACCUUAAGGGAAGGUCAAGAU | | 1721-1743 | AUCUUGACCUUCCCUUAAGGUAC | |
| AD-1657299 | GUCAAGAGGGAGAUGGGCAAU | | 1734-1756 | AUUGCCCAUCUCCCUCUUGACCU | |
| AD-1657322 | GCUGAGGGAGGAUGCUUAGGU | | 1757-1779 | ACCUAAGCAUCCUCCCUCAGCGC | |
| AD-1657324 | AGAAACAGGAGUCAGGAAAAU | | 1785-1807 | AUUUUCCUGACUCCUGUUUCUGG | |
| AD-1657335 | UCAGGAAAAUGAGGCACUAAU | | 1796-1818 | AUUAGUGCCUCAUUUUCCUGACU | |
| AD-1657347 | GGCACUAAGCCUAAGAAGUUU | | 1808-1830 | AAACUUCUUAGGCUUAGUGCCUC | |
| AD-1657359 | AAGAAGUUCCCUGGUUUUUCU | | 1820-1842 | AGAAAAACCAGGGAACUUCUUAG | |
| AD-1657374 | CACUGGGAGACAAGCAUUUAU | | 1855-1877 | AUAAAUGCUUGUCUCCCAGUGGG | |
| AD-1657385 | AAGCAUUUAUACUUUCUUUCU | | 1866-1888 | AGAAAGAAAGUAUAAAUGCUUGU | |
| AD-1657395 | UUUUGAGAUCGAGUCUCGCUU | | 1899-1921 | AAGCGAGACUCGAUCUCAAAAAA | |
| AD-1657410 | UCUCGCUCUGUCACCAGGCUU | | 1912-1934 | AAGCCUGGUGACAGAGCGAGACU | |
| AD-1657431 | AGUGCAGUGACACGAUCUUGU | | 1934-1956 | ACAAGAUCGUGUCACUGCACUCC | |
| AD-1657446 | GCUCACUGCAACCUCCGUCUU | | 1954-1976 | AAGACGGAGGUUGCAGUGAGCCA | |
| AD-1657457 | CCUCCGUCUCCUGGGUUCAAU | | 1965-1987 | AUUGAACCCAGGAGACGGAGGUU | |
| AD-1657463 | UUCUGCCUCAGCCUCCCGAGU | | 1992-2014 | ACUCGGGAGGCUGAGGCAGAAGA | |
| AD-1657475 | UGGGUUCAAGUGAUUCUUCUU | | 1976-1998 | AAGAAGAAUCACUUGAACCCAGG | |
| AD-1657503 | GAGCAGCUGGGAUUACAGGCU | | 2009-2031 | AGCCUGUAAUCCCAGCUGCUCGG | |
| AD-1657520 | CGCCCACUAAUUUUUGUAUUU | | 2028-2050 | AAAUACAAAAAUUAGUGGGCGCC | |
| AD-1657529 | UGUAUUCUUAGUAGAAACGAU | | 2042-2064 | AUCGUUUCUACUAAGAAUACAAA | |
| AD-1657540 | UAGAAACGAGGUUUCAACAUU | | 2053-2075 | AAUGUUGAAACCUCGUUUCUACU | |
| AD-1657552 | UUCAACAUGUUGGCCAGGAUU | | 2065-2087 | AAUCCUGGCCAACAUGUUGAAAC | |
| AD-1657564 | CCAGGAUGGUCUCAAUCUCUU | | 2078-2100 | AAGAGAUUGAGACCAUCCUGGCC | |
| AD-1657575 | UCAAUCUCUUGACCUCUUGAU | | 2089-2111 | AUCAAGAGGUCAAGAGAUUGAGA | |
| AD-1657586 | ACCUCUUGAUCCACCCGACUU | | 2100-2122 | AAGUCGGGUGGAUCAAGAGGUCA | |
| AD-1657600 | CCGACUUGGCCUCCCGAAGUU | | 2114-2136 | AACUUCGGGAGGCCAAGUCGGGU | |
| AD-1657615 | GAAGUGAUGAGAUUAUAGGCU | | 2129-2151 | AGCCUAUAAUCUCAUCACUUCGG | |
| AD-1657641 | CCUGGCUUAUACUUUCUUAAU | | 2162-2184 | AUUAAGAAAGUAUAAGCCAGGCG | |
| AD-1657653 | GAGAAAGAAAAUCAACAAAUU | | 2189-2211 | AAUUUGUUGAUUUUCUUUCUCCU | |
| AD-1657674 | UGAGUCAUAAAGAAGGGUUAU | | 2210-2232 | AUAACCCUUCUUUAUGACUCACA | |
| AD-1657687 | AGGGUUAGGGUGAUGGUCCAU | | 2223-2245 | AUGGACCAUCACCCUAACCCUUC | |
| AD-1657704 | CCAGAGCAACAGUUCUUCAAU | | 2240-2262 | AUUGAAGAACUGUUGCUCUGGAC | |
| AD-1657716 | UUCUUCAAGUGUACUCUGUAU | | 2252-2274 | AUACAGAGUACACUUGAAGAACU | |
| AD-1657727 | UACUCUGUAGGCUUCUGGGAU | | 2263-2285 | AUCCCAGAAGCCUACAGAGUACA | |
| AD-1657741 | CUGGGAGGUCCCUUUUCAGGU | | 2277-2299 | ACCUGAAAAGGGACCUCCCAGAA | |
| AD-1657744 | GUCCACAAAGUCAAAGCUAUU | | 2300-2322 | AAUAGCUUUGACUUUGUGGACAC | |
| AD-1657760 | CUAACAUGUUAUUUGCCUUUU | | 2333-2355 | AAAAGGCAAAUAACAUGUUAGUA | |
| AD-1657776 | CUUUUGAAUUCUCAUUAUCUU | | 2349-2371 | AAGAUAAUGAGAAUUCAAAAGGC | |
| AD-1657793 | GUAUUGUGGAGUUUUCCAGAU | | 2376-2398 | AUCUGGAAAACUCCACAAUACAA | |
| AD-1657811 | GAGGCCGUGUGACAUGUGAUU | | 2394-2416 | AAUCACAUGUCACACGGCCUCUG | |
| AD-1657822 | ACAUGUGAUUACAUCAUCUUU | | 2405-2427 | AAAGAUGAUGUAAUCACAUGUCA | |
| AD-1657834 | AUCAUCUUUCUGACAUCAUUU | | 2417-2439 | AAAUGAUGUCAGAAAGAUGAUGU | |
| AD-1657845 | GACAUCAUUGUUAAUGGAAUU | | 2428-2450 | AAUUCCAUUAACAAUGAUGUCAG | |

TABLE 3
Modified Sense and Antisense Strand Sequences
of INHBE dsRNA Agents

| | | SEQ | Antisense | SEQ | | SEQ |
|---|---|---|---|---|---|---|
| Duplex | Sense Sequence | ID | Sequence | ID | mRNA target | ID |
| Name | 5′ to 3′ | NO: | 5′ to 3′ | NO: | sequence | NO: |
| AD-1656008 | usasgccaGfaCfAf | | asUfscacAfgCfUf | | AGTAGCCAGACATGA | |
| | UfgagcugugauL96 | | caugUfcUfggcuas | | GCTGTGAG | |
| | | | csu | | | |
| AD-1656026 | gsasggguCfaAfGf | | asGfsauaGfcUfGf | | GTGAGGGTCAAGCAC | |
| | CfacagcuaucuL96 | | ugcuUfgAfcccucs | | AGCTATCC | |
| | | | asc | | | |
| AD-1656043 | asusccauCfaGfAf | | asAfsaguAfgAfUf | | CTATCCATCAGATGA | |
| | UfgaucuacuuuL96 | | caucUfgAfuggaus | | TCTACTTT | |
| | | | asg | | | |
| AD-1656054 | gsasucuaCfuUfUf | | asAfsggaAfgGfCf | | ATGATCTACTTTCAG | |
| | CfagccuuccuuL96 | | ugaaAfgUfagaucs | | CCTTCCTG | |
| | | | asu | | | |
| AD-1656074 | gsasguccCfaGfAf | | asUfscuuCfuAfUf | | CTGAGTCCCAGACAA | |
| | CfaauagaagauL96 | | ugucUfgGfgacucs | | TAGAAGAC | |
| | | | asg | | | |
| AD-1656086 | asusagaaGfaCfAf | | asUfsacaGfcCfAf | | CAATAGAAGACAGGT | |
| | GfguggcuguauL96 | | ccugUfcUfucuaus | | GGCTGTAC | |
| | | | usg | | | |
| AD-1656097 | gsusggcuGfuAfCf | | asUfsuggCfcAfAf | | AGGTGGCTGTACCCT | |
| | CfcuuggccaauL96 | | ggguAfcAfgccacs | | TGGCCAAG | |
| | | | csu | | | |
| AD-1656108 | csusuggcCfaAfGf | | asCfsacaCfcUfAf | | CCCTTGGCCAAGGGT | |
| | GfguagguguguL96 | | cccuUfgGfccaags | | AGGTGTGG | |
| | | | gsg | | | |
| AD-1656125 | gsusggcaGfuGfGf | | asAfscagCfaGfAf | | GTGTGGCAGTGGTGT | |
| | UfgucugcuguuL96 | | caccAfcUfgccacs | | CTGCTGTC | |
| | | | asc | | | |
| AD-1656139 | usgscuguCfaCfUf | | asAfsugaGfgGfCf | | TCTGCTGTCACTGTG | |
| | GfugcccucauuL96 | | acagUfgAfcagcas | | CCCTCATT | |
| | | | gsa | | | |
| AD-1656146 | asgscaauCfaGfAf | | asGfsucuGfuUfGf | | CCAGCAATCAGACTC | |
| | CfucaacagacuL96 | | agucUfgAfuugcus | | AACAGACG | |
| | | | gsg | | | |
| AD-1656164 | ascsggagCfaAfCf | | asUfscggAfuGfGf | | AGACGGAGCAACTGC | |
| | UfgccauccgauL96 | | caguUfgCfuccgus | | CATCCGAG | |
| | | | csu | | | |
| AD-1656185 | gscsuccuGfaAfCf | | asAfsaugGfcCfCf | | AGGCTCCTGAACCAG | |
| | CfagggccauuuL96 | | ugguUfcAfggagcs | | GGCCATTC | |
| | | | csu | | | |
| AD-1656196 | asgsggccAfuUfCf | | asUfsgcuCfcUfGf | | CCAGGGCCATTCACC | |
| | AfccaggagcauL96 | | gugaAfuGfgcccus | | AGGAGCAT | |
| | | | gsg | | | |
| AD-1656220 | gscsucccUfgAfUf | | asAfsgagCfuGfGf | | CGGCTCCCTGATGTC | |
| | GfuccagcucuuL96 | | acauCfaGfggagcs | | CAGCTCTG | |
| | | | csg | | | |
| AD-1656233 | csasgcucUfgGfCf | | asAfsgcaGfcAfCf | | TCCAGCTCTGGCTGG | |
| | UfggugcugcuuL96 | | cagcCfaGfagcugs | | TGCTGCTG | |
| | | | gsa | | | |
| AD-1656254 | usgsggcaCfuGfGf | | asUfsgugCfuCfGf | | TGTGGGCACTGGTGC | |
| | UfgcgagcacauL96 | | caccAfgUfgcccas | | GAGCACAG | |
| | | | csa | | | |
| AD-1656260 | asgsggucUfgUfGf | | asAfsggaGfgGfAf | | ACAGGGTCTGTGTGT | |
| | UfgucccuccuuL96 | | cacaCfaGfacccus | | CCCTCCTG | |
| | | | gsu | | | |
| AD-1656265 | csasagcaGfaAfCf | | asAfsccaGfaGfCf | | CCCAAGCAGAACGAG | |
| | GfagcucugguuL96 | | ucguUfcUfgcuugs | | CTCTGGTG | |
| | | | gsg | | | |
| AD-1656280 | csusggugCfuGfGf | | asUfsuggCfuAfGf | | CTCTGGTGCTGGAGC | |
| | AfgcuagccaauL96 | | cuccAfgCfaccags | | TAGCCAAG | |
| | | | asg | | | |
| AD-1656292 | csusagccAfaGfCf | | asAfsggaUfuUfGf | | AGCTAGCCAAGCAGC | |
| | AfgcaaauccuuL96 | | cugcUfuGfgcuags | | AAATCCTG | |
| | | | csu | | | |
| AD-1656307 | asusccugGfaUfGf | | asAfsgguGfcAfAf | | AAATCCTGGATGGGT | |
| | GfguugcaccuuL96 | | cccaUfcCfaggaus | | TGCACCTG | |
| | | | usu | | | |
| AD-1656319 | ususgcacCfuGfAf | | asGfsgacGfaCfUf | | GGTTGCACCTGACCA | |
| | CfcagucguccuL96 | | ggucAfgGfugcaas | | GTCGTCCC | |
| | | | csc | | | |
| AD-1656333 | uscsguccCfaGfAf | | asGfsaugAfgUfUf | | AGTCGTCCCAGAATA | |
| | AfuaacucaucuL96 | | auucUfgGfgacgas | | ACTCATCC | |
| | | | csu | | | |
| AD-1656360 | cscscuccGfgAfGf | | asUfsggcUfgUfAf | | AGCCCTCCGGAGACT | |
| | AfcuacagccauL96 | | gucuCfcGfgagggs | | ACAGCCAG | |
| | | | csu | | | |
| AD-1656372 | usascagcCfaGfGf | | asAfsgccAfcAfCf | | ACTACAGCCAGGGAG | |
| | GfaguguggcuuL96 | | ucccUfgGfcuguas | | TGTGGCTC | |
| | | | gsu | | | |
| AD-1656383 | asgsugugGfcUfCf | | asCfscauUfcCfCf | | GGAGTGTGGCTCCAG | |
| | CfagggaaugguL96 | | uggaGfcCfacacus | | GGAATGGG | |
| | | | csc | | | |
| AD-1656392 | csasucagCfuUfUf | | asUfsgacAfgUfAf | | GTCATCAGCTTTGCT | |
| | GfcuacugucauL96 | | gcaaAfgCfugaugs | | ACTGTCAC | |
| | | | asc | | | |
| AD-1656406 | csusgucaCfaGfAf | | asUfsgaaGfuGfGf | | TACTGTCACAGACTC | |
| | CfuccacuucauL96 | | agucUfgUfgacags | | CACTTCAG | |
| | | | usa | | | |
| AD-1656417 | uscscacuUfcAfGf | | asGfsagcUfgUfAf | | ACTCCACTTCAGCCT | |
| | CfcuacagcucuL96 | | ggcuGfaAfguggas | | ACAGCTCC | |
| | | | gsu | | | |
| AD-1656437 | cscsugcuCfaCfUf | | asAfscagGfuGfAf | | TCCCTGCTCACTTTT | |
| | UfuucaccuguuL96 | | aaagUfgAfgcaggs | | CACCTGTC | |
| | | | gsa | | | |
| AD-1656449 | uscsaccuGfuCfCf | | asAfsccgAfgGfAf | | TTTCACCTGTCCACT | |
| | AfcuccucgguuL96 | | guggAfcAfggugas | | CCTCGGTC | |
| | | | asa | | | |
| AD-1656468 | uscsccacCfaCfCf | | asGfscauGfgUfAf | | GGTCCCACCACCTGT | |
| | UfguaccaugcuL96 | | caggUfgGfugggas | | ACCATGCC | |
| | | | csc | | | |
| AD-1656480 | usasccauGfcCfCf | | asAfsgccAfcAfGf | | TGTACCATGCCCGCC | |
| | GfccuguggcuuL96 | | gcggGfcAfugguas | | TGTGGCTG | |
| | | | csa | | | |
| AD-1656492 | cscscuucCfuGfGf | | asGfscaaAfgAfGf | | CACCCTTCCTGGCAC | |
| | CfacucuuugcuL96 | | ugccAfgGfaagggs | | TCTTTGCT | |
| | | | usg | | | |
| AD-1656504 | csuscuuuGfcUfUf | | asGfsaagAfuCfCf | | CACTCTTTGCTTGAG | |
| | GfaggaucuucuL96 | | ucaaGfcAfaagags | | GATCTTCC | |
| | | | usg | | | |
| AD-1656535 | ascsucucCfuGfGf | | asUfsgguGfcUfCf | | GCACTCTCCTGGCTG | |
| | CfugagcaccauL96 | | agccAfgGfagagus | | AGCACCAC | |
| | | | gsc | | | |
| AD-1656547 | gsasgcacCfaCfAf | | asAfsgguUfgGfUf | | CTGAGCACCACATCA | |
| | UfcaccaaccuuL96 | | gaugUfgGfugcucs | | CCAACCTG | |
| | | | asg | | | |
| AD-1656559 | ascscaacCfuGfGf | | asGfsuauGfcCfAf | | TCACCAACCTGGGCT | |
| | GfcuggcauacuL96 | | gcccAfgGfuuggus | | GGCATACC | |
| | | | gsa | | | |
| AD-1656570 | csusggcaUfaCfCf | | asGfscagAfgUfUf | | GGCTGGCATACCTTA | |
| | UfuaacucugcuL96 | | aaggUfaUfgccags | | ACTCTGCC | |
| | | | csc | | | |
| AD-1656587 | usgscccuCfuAfGf | | asCfscucAfaGfCf | | TCTGCCCTCTAGTGG | |
| | UfggcuugagguL96 | | cacuAfgAfgggcas | | CTTGAGGG | |
| | | | gsa | | | |
| AD-1656591 | asgsaaguCfuGfGf | | asUfsuucAfgGfAf | | TGAGAAGTCTGGTGT | |
| | UfguccugaaauL96 | | caccAfgAfcuucus | | CCTGAAAC | |
| | | | csa | | | |
| AD-1656602 | gsusccugAfaAfCf | | asUfscuaGfuUfGf | | GTGTCCTGAAACTGC | |
| | UfgcaacuagauL96 | | caguUfuCfaggacs | | AACTAGAC | |
| | | | asc | | | |
| AD-1656622 | ascsagcaCfaGfUf | | asUfsuguCfcAfGf | | CAACAGCACAGTTAC | |
| | UfacuggacaauL96 | | uaacUfgUfgcugus | | TGGACAAC | |
| | | | usg | | | |
| AD-1656634 | csusggacAfaCfCf | | asGfsagcCfgCfCf | | TACTGGACAACCGAG | |
| | GfaggcggcucuL96 | | ucggUfuGfuccags | | GCGGCTCT | |
| | | | usa | | | |
| AD-1656647 | gscsggcuCfuUfGf | | asCfsugcUfgUfGf | | AGGCGGCTCTTGGAC | |
| | GfacacagcaguL96 | | uccaAfgAfgccgcs | | ACAGCAGG | |
| | | | csu | | | |
| AD-1656667 | gsascaccAfgCfAf | | asUfsaggAfaGfGf | | AGGACACCAGCAGCC | |
| | GfcccuuccuauL96 | | gcugCfuGfgugucs | | CTTCCTAG | |
| | | | csu | | | |
| AD-1656679 | cscsuuccUfaGfAf | | asGfsaucUfuAfAf | | GCCCTTCCTAGAGCT | |
| | GfcuuaagaucuL96 | | gcucUfaGfgaaggs | | TAAGATCC | |
| | | | gsc | | | |
| AD-1656690 | csusuaagAfuCfCf | | asUfscauUfgGfCf | | AGCTTAAGATCCGAG | |
| | GfagccaaugauL96 | | ucggAfuCfuuaags | | CCAATGAG | |
| | | | csu | | | |
| AD-1656701 | asgsccaaUfgAfGf | | asCfsugcUfcCfAf | | CGAGCCAATGAGCCT | |
| | CfcuggagcaguL96 | | ggcuCfaUfuggcus | | GGAGCAGG | |
| | | | csg | | | |
| AD-1656716 | asgsgcgaGfaCfCf | | asUfscuaCfgUfAf | | GCAGGCGAGACCATT | |
| | AfuuacguagauL96 | | auggUfcUfcgccus | | ACGTAGAC | |
| | | | gsc | | | |
| AD-1656728 | usascguaGfaCfUf | | asAfsguuCfcUfGf | | ATTACGTAGACTTCC | |
| | UfccaggaacuuL96 | | gaagUfcUfacguas | | AGGAACTG | |
| | | | asu | | | |
| AD-1656740 | csasggaaCfuGfGf | | asUfscccGfcCfAf | | TCCAGGAACTGGGAT | |
| | GfauggcgggauL96 | | ucccAfgUfuccugs | | GGCGGGAC | |
| | | | gsa | | | |
| AD-1656754 | gscsgggaCfuGfGf | | asGfscugCfaGfUf | | TGGCGGGACTGGATA | |
| | AfuacugcagcuL96 | | auccAfgUfcccgcs | | CTGCAGCC | |
| | | | csa | | | |
| AD-1656762 | usasccagCfuGfAf | | asCfsugcAfgUfAf | | GGTACCAGCTGAATT | |
| | AfuuacugcaguL96 | | auucAfgCfugguas | | ACTGCAGT | |
| | | | csc | | | |
| AD-1656775 | ascsugcaGfuGfGf | | asAfsgggCfaCfUf | | TTACTGCAGTGGGCA | |
| | GfcagugcccuuL96 | | gcccAfcUfgcagus | | GTGCCCTC | |
| | | | asa | | | |
| AD-1656792 | csasggcaUfuGfCf | | asGfsaaaGfaGfGf | | CCCAGGCATTGCTGC | |
| | UfgccucuuucuL96 | | cagcAfaUfgccugs | | CTCTTTCC | |
| | | | gsg | | | |
| AD-1656808 | ususuccaUfuCfUf | | asUfsgaaGfaCfGf | | TCTTTCCATTCTGCC | |
| | GfccgucuucauL96 | | gcagAfaUfggaaas | | GTCTTCAG | |
| | | | gsa | | | |
| AD-1656820 | csgsucuuCfaGfCf | | asCfsuuuGfaGfGf | | GCCGTCTTCAGCCTC | |
| | CfuccucaaaguL96 | | aggcUfgAfagacgs | | CTCAAAGC | |
| | | | gsc | | | |
| AD-1656832 | cscsucaaAfgCfCf | | asAfsaggAfuUfGf | | CTCCTCAAAGCCAAC | |
| | AfacaauccuuuL96 | | uuggCfuUfugaggs | | AATCCTTG | |
| | | | asg | | | |
| AD-1656849 | csusuggcCfuGfCf | | asGfsgagGfuAfCf | | TCCTTGGCCTGCCAG | |
| | CfaguaccuccuL96 | | uggcAfgGfccaags | | TACCTCCT | |
| | | | gsa | | | |
| AD-1656862 | usasccucCfuGfUf | | asUfsaggGfaCfAf | | AGTACCTCCTGTTGT | |
| | UfgugucccuauL96 | | caacAfgGfagguas | | GTCCCTAC | |
| | | | csu | | | |
| AD-1656873 | gsusguccCfuAfCf | | asCfscuuCfgGfGf | | TTGTGTCCCTACTGC | |
| | UfgcccgaagguL96 | | caguAfgGfgacacs | | CCGAAGGC | |
| | | | asa | | | |
| AD-1656876 | csuscucuCfuCfCf | | asUfsccaGfgUfAf | | CCCTCTCTCTCCTCT | |
| | UfcuaccuggauL96 | | gaggAfgAfgagags | | ACCTGGAT | |
| | | | gsg | | | |
| AD-1656888 | usasccugGfaUfCf | | asUfsugcCfaUfUf | | TCTACCTGGATCATA | |
| | AfuaauggcaauL96 | | augaUfcCfagguas | | ATGGCAAT | |
| | | | gsa | | | |
| AD-1656900 | asasuggcAfaUfGf | | asGfsucuUfgAfCf | | ATAATGGCAATGTGG | |
| | UfggucaagacuL96 | | cacaUfuGfccauus | | TCAAGACG | |
| | | | asu | | | |
| AD-1656915 | asasgacgGfaUfGf | | asAfsuauCfuGfGf | | TCAAGACGGATGTGC | |
| | UfgccagauauuL96 | | cacaUfcCfgucuus | | CAGATATG | |
| | | | gsa | | | |
| AD-1656926 | gscscagaUfaUfGf | | asCfscucCfaCfCf | | GTGCCAGATATGGTG | |
| | GfugguggagguL96 | | accaUfaUfcuggcs | | GTGGAGGC | |
| | | | asc | | | |
| AD-1656942 | gsasggccUfgUfGf | | asUfsagcUfgCfAf | | TGGAGGCCTGTGGCT | |
| | GfcugcagcuauL96 | | gccaCfaGfgccucs | | GCAGCTAG | |
| | | | csa | | | |
| AD-1656954 | usgscagcUfaGfCf | | asAfsgguCfcUfCf | | GCTGCAGCTAGCAAG | |
| | AfagaggaccuuL96 | | uugcUfaGfcugcas | | AGGACCTG | |
| | | | gsc | | | |
| AD-1656958 | csusuuggAfgUfGf | | asUfsuggUfcUfCf | | GGCTTTGGAGTGAAG | |
| | AfagagaccaauL96 | | uucaCfuCfcaaags | | AGACCAAG | |
| | | | csc | | | |
| AD-1656969 | asgsagacCfaAfGf | | asGfsaaaCfuUfCf | | GAAGAGACCAAGATG | |
| | AfugaaguuucuL96 | | aucuUfgGfucucus | | AAGTTTCC | |
| | | | usc | | | |
| AD-1656996 | csasgggcAfuCfUf | | asCfsuccAfgUfCf | | CACAGGGCATCTGTG | |
| | GfugacuggaguL96 | | acagAfuGfcccugs | | ACTGGAGG | |
| | | | usg | | | |
| AD-1657013 | gsasggcaUfcAfGf | | asGfsaucAfgGfAf | | TGGAGGCATCAGATT | |
| | AfuuccugaucuL96 | | aucuGfaUfgccucs | | CCTGATCC | |
| | | | csa | | | |
| AD-1657022 | ascsccaaCfaAfCf | | asUfsugcCfaGfGf | | CAACCCAACAACCAC | |
| | CfaccuggcaauL96 | | ugguUfgUfugggus | | CTGGCAAT | |
| | | | usg | | | |
| AD-1657037 | gsgscaauAfuGfAf | | asGfsucaAfgUfGf | | CTGGCAATATGACTC | |
| | CfucacuugacuL96 | | agucAfuAfuugccs | | ACTTGACC | |
| | | | asg | | | |
| AD-1657045 | gsascccaAfaUfGf | | asAfsgaaAfgUfGf | | GGGACCCAAATGGGC | |
| | GfgcacuuucuuL96 | | cccaUfuUfgggucs | | ACTTTCTT | |
| | | | csc | | | |
| AD-1657058 | ascsuuucUfuGfUf | | asAfsgagUfcUfCf | | GCACTTTCTTGTCTG | |
| | CfugagacucuuL96 | | agacAfaGfaaagus | | AGACTCTG | |
| | | | gsc | | | |
| AD-1657069 | usgsagacUfcUfGf | | asUfsggaAfuAfAf | | TCTGAGACTCTGGCT | |
| | GfcuuauuccauL96 | | gccaGfaGfucucas | | TATTCCAG | |
| | | | gsa | | | |
| AD-1657085 | uscscaggUfuGfGf | | asAfsacaCfaUfCf | | ATTCCAGGTTGGCTG | |
| | CfugauguguuuL96 | | agccAfaCfcuggas | | ATGTGTTG | |
| | | | asu | | | |
| AD-1657099 | usgsuguuGfgGfAf | | asUfsuuaCfcCfAf | | GATGTGTTGGGAGAT | |
| | GfauggguaaauL96 | | ucucCfcAfacacas | | GGGTAAAG | |
| | | | usc | | | |
| AD-1657113 | gsgsuaaaGfcGfUf | | asUfsuuaGfaAfGf | | TGGGTAAAGCGTTTC | |
| | UfucuucuaaauL96 | | aaacGfcUfuuaccs | | TTCTAAAG | |
| | | | csa | | | |
| AD-1657119 | usascccaGfaAfAf | | asGfsaaaUfcAfUf | | TCTACCCAGAAAGCA | |
| | GfcaugauuucuL96 | | gcuuUfcUfggguas | | TGATTTCC | |
| | | | gsa | | | |
| AD-1657133 | gsasuuucCfuGfCf | | asAfsggaCfuUfAf | | ATGATTTCCTGCCCT | |
| | CfcuaaguccuuL96 | | gggcAfgGfaaaucs | | AAGTCCTG | |
| | | | asu | | | |
| AD-1657147 | asgsuccuGfuGfAf | | asUfsgacAfuCfUf | | TAAGTCCTGTGAGAA | |
| | GfaagaugucauL96 | | ucucAfcAfggacus | | GATGTCAG | |
| | | | usa | | | |
| AD-1657164 | uscsagggAfcUfAf | | asCfscucCfcUfCf | | TGTCAGGGACTAGGG | |
| | GfggagggagguL96 | | ccuaGfuCfccugas | | AGGGAGGG | |
| | | | csa | | | |
| AD-1657185 | asasuuacUfuAfGf | | asUfsuggGfaGfAf | | AAAATTACTTAGCCT | |
| | CfcucucccaauL96 | | ggcuAfaGfuaauus | | CTCCCAAG | |
| | | | usu | | | |
| AD-1657202 | csasagauGfaGfAf | | asUfsugaGfgAfCf | | CCCAAGATGAGAAAG | |
| | AfaguccucaauL96 | | uuucUfcAfucuugs | | TCCTCAAG | |
| | | | gsg | | | |
| AD-1657211 | gsgsaggaAfgCfAf | | asAfsccaUfcUfAf | | GAGGAGGAAGCAGAT | |
| | GfauagaugguuL96 | | ucugCfuUfccuccs | | AGATGGTC | |
| | | | usc | | | |
| AD-1657234 | gscsaggcUfuGfAf | | asUfsuacCfcUfGf | | CAGCAGGCTTGAAGC | |
| | AfgcaggguaauL96 | | cuucAfaGfccugcs | | AGGGTAAG | |
| | | | usg | | | |
| AD-1657245 | gscsagggUfaAfGf | | asGfsgccAfgCfCf | | AAGCAGGGTAAGCAG | |
| | CfaggcuggccuL96 | | ugcuUfaCfccugcs | | GCTGGCCC | |
| | | | usu | | | |
| AD-1657261 | gsgscccaGfgGfUf | | asAfsacaGfcCfCf | | CTGGCCCAGGGTAAG | |
| | AfagggcuguuuL96 | | uuacCfcUfgggccs | | GGCTGTTG | |
| | | | asg | | | |
| AD-1657274 | gsgscuguUfgAfGf | | asCfsuuaAfgGfUf | | AGGGCTGTTGAGGTA | |
| | GfuaccuuaaguL96 | | accuCfaAfcagccs | | CCTTAAGG | |
| | | | csu | | | |
| AD-1657286 | ascscuuaAfgGfGf | | asUfscuuGfaCfCf | | GTACCTTAAGGGAAG | |
| | AfaggucaagauL96 | | uuccCfuUfaaggus | | GTCAAGAG | |
| | | | asc | | | |
| AD-1657299 | gsuscaagAfgGfGf | | asUfsugcCfcAfUf | | AGGTCAAGAGGGAGA | |
| | AfgaugggcaauL96 | | cuccCfuCfuugacs | | TGGGCAAG | |
| | | | csu | | | |
| AD-1657322 | gscsugagGfgAfGf | | asCfscuaAfgCfAf | | GCGCTGAGGGAGGAT | |
| | GfaugcuuagguL96 | | uccuCfcCfucagcs | | GCTTAGGG | |
| | | | gsc | | | |
| AD-1657324 | asgsaaacAfgGfAf | | asUfsuuuCfcUfGf | | CCAGAAACAGGAGTC | |
| | GfucaggaaaauL96 | | acucCfuGfuuucus | | AGGAAAAT | |
| | | | gsg | | | |
| AD-1657335 | uscsaggaAfaAfUf | | asUfsuagUfgCfCf | | AGTCAGGAAAATGAG | |
| | GfaggcacuaauL96 | | ucauUfuUfccugas | | GCACTAAG | |
| | | | csu | | | |
| AD-1657347 | gsgscacuAfaGfCf | | asAfsacuUfcUfUf | | GAGGCACTAAGCCTA | |
| | CfuaagaaguuuL96 | | aggcUfuAfgugccs | | AGAAGTTC | |
| | | | usc | | | |
| AD-1657359 | asasgaagUfuCfCf | | asGfsaaaAfaCfCf | | CTAAGAAGTTCCCTG | |
| | CfugguuuuucuL96 | | agggAfaCfuucuus | | GTTTTTCC | |
| | | | asg | | | |
| AD-1657374 | csascuggGfaGfAf | | asUfsaaaUfgCfUf | | CCCACTGGGAGACAA | |
| | CfaagcauuuauL96 | | ugucUfcCfcagugs | | GCATTTAT | |
| | | | gsg | | | |
| AD-1657385 | asasgcauUfuAfUf | | asGfsaaaGfaAfAf | | ACAAGCATTTATACT | |
| | AfcuuucuuucuL96 | | guauAfaAfugcuus | | TTCTTTCT | |
| | | | gsu | | | |
| AD-1657395 | ususuugaGfaUfCf | | asAfsgcgAfgAfCf | | TTTTTTGAGATCGAG | |
| | GfagucucgcuuL96 | | ucgaUfcUfcaaaas | | TCTCGCTC | |
| | | | asa | | | |
| AD-1657410 | uscsucgcUfcUfGf | | asAfsgccUfgGfUf | | AGTCTCGCTCTGTCA | |
| | UfcaccaggcuuL96 | | gacaGfaGfcgagas | | CCAGGCTG | |
| | | | csu | | | |
| AD-1657431 | asgsugcaGfuGfAf | | asCfsaagAfuCfGf | | GGAGTGCAGTGACAC | |
| | CfacgaucuuguL96 | | ugucAfcUfgcacus | | GATCTTGG | |
| | | | csc | | | |
| AD-1657446 | gscsucacUfgCfAf | | asAfsgacGfgAfGf | | TGGCTCACTGCAACC | |
| | AfccuccgucuuL96 | | guugCfaGfugagcs | | TCCGTCTC | |
| | | | csa | | | |
| AD-1657457 | cscsuccgUfcUfCf | | asUfsugaAfcCfCf | | AACCTCCGTCTCCTG | |
| | CfuggguucaauL96 | | aggaGfaCfggaggs | | GGTTCAAG | |
| | | | usu | | | |
| AD-1657463 | ususcugcCfuCfAf | | asCfsucgGfgAfGf | | TCTTCTGCCTCAGCC | |
| | GfccucccgaguL96 | | gcugAfgGfcagaas | | TCCCGAGC | |
| | | | gsa | | | |
| AD-1657475 | usgsgguuCfaAfGf | | asAfsgaaGfaAfUf | | CCTGGGTTCAAGTGA | |
| | UfgauucuucuuL96 | | cacuUfgAfacccas | | TTCTTCTG | |
| | | | gsg | | | |
| AD-1657503 | gsasgcagCfuGfGf | | asGfsccuGfuAfAf | | CCGAGCAGCTGGGAT | |
| | GfauuacaggcuL96 | | ucccAfgCfugcucs | | TACAGGCG | |
| | | | gsg | | | |
| AD-1657520 | csgscccaCfuAfAf | | asAfsauaCfaAfAf | | GGCGCCCACTAATTT | |
| | UfuuuuguauuuL96 | | aauuAfgUfgggcgs | | TTGTATTC | |
| | | | csc | | | |
| AD-1657529 | usgsuauuCfuUfAf | | asUfscguUfuCfUf | | TTTGTATTCTTAGTA | |
| | GfuagaaacgauL96 | | acuaAfgAfauacas | | GAAACGAG | |
| | | | asa | | | |
| AD-1657540 | usasgaaaCfgAfGf | | asAfsuguUfgAfAf | | AGTAGAAACGAGGTT | |
| | GfuuucaacauuL96 | | accuCfgUfuucuas | | TCAACATG | |
| | | | csu | | | |
| AD-1657552 | ususcaacAfuGfUf | | asAfsuccUfgGfCf | | GTTTCAACATGTTGG | |
| | UfggccaggauuL96 | | caacAfuGfuugaas | | CCAGGATG | |
| | | | asc | | | |
| AD-1657564 | cscsaggaUfgGfUf | | asAfsgagAfuUfGf | | GGCCAGGATGGTCTC | |
| | CfucaaucucuuL96 | | agacCfaUfccuggs | | AATCTCTT | |
| | | | csc | | | |
| AD-1657575 | uscsaaucUfcUfUf | | asUfscaaGfaGfGf | | TCTCAATCTCTTGAC | |
| | GfaccucuugauL96 | | ucaaGfaGfauugas | | CTCTTGAT | |
| | | | gsa | | | |
| AD-1657586 | ascscucuUfgAfUf | | asAfsgucGfgGfUf | | TGACCTCTTGATCCA | |
| | CfcacccgacuuL96 | | ggauCfaAfgaggus | | CCCGACTT | |
| | | | csa | | | |
| AD-1657600 | cscsgacuUfgGfCf | | asAfscuuCfgGfGf | | ACCCGACTTGGCCTC | |
| | CfucccgaaguuL96 | | aggcCfaAfgucggs | | CCGAAGTG | |
| | | | gsu | | | |
| AD-1657615 | gsasagugAfuGfAf | | asGfsccuAfuAfAf | | CCGAAGTGATGAGAT | |
| | GfauuauaggcuL96 | | ucucAfuCfacuucs | | TATAGGCG | |
| | | | gsg | | | |
| AD-1657641 | cscsuggcUfuAfUf | | asUfsuaaGfaAfAf | | CGCCTGGCTTATACT | |
| | AfcuuucuuaauL96 | | guauAfaGfccaggs | | TTCTTAAT | |
| | | | csg | | | |
| AD-1657653 | gsasgaaaGfaAfAf | | asAfsuuuGfuUfGf | | AGGAGAAAGAAAATC | |
| | AfucaacaaauuL96 | | auuuUfcUfuucucs | | AACAAATG | |
| | | | csu | | | |
| AD-1657674 | usgsagucAfuAfAf | | asUfsaacCfcUfUf | | TGTGAGTCATAAAGA | |
| | AfgaaggguuauL96 | | cuuuAfuGfacucas | | AGGGTTAG | |
| | | | csa | | | |
| AD-1657687 | asgsgguuAfgGfGf | | asUfsggaCfcAfUf | | GAAGGGTTAGGGTGA | |
| | UfgaugguccauL96 | | caccCfuAfacccus | | TGGTCCAG | |
| | | | usc | | | |
| AD-1657704 | cscsagagCfaAfCf | | asUfsugaAfgAfAf | | GTCCAGAGCAACAGT | |
| | AfguucuucaauL96 | | cuguUfgCfucuggs | | TCTTCAAG | |
| | | | asc | | | |
| AD-1657716 | ususcuucAfaGfUf | | asUfsacaGfaGfUf | | AGTTCTTCAAGTGTA | |
| | GfuacucuguauL96 | | acacUfuGfaagaas | | CTCTGTAG | |
| | | | csu | | | |
| AD-1657727 | usascucuGfuAfGf | | asUfscccAfgAfAf | | TGTACTCTGTAGGCT | |
| | GfcuucugggauL96 | | gccuAfcAfgaguas | | TCTGGGAG | |
| | | | csa | | | |
| AD-1657741 | csusgggaGfgUfCf | | asCfscugAfaAfAf | | TTCTGGGAGGTCCCT | |
| | CfcuuuucagguL96 | | gggaCfcUfcccags | | TTTCAGGG | |
| | | | asa | | | |
| AD-1657744 | gsusccacAfaAfGf | | asAfsuagCfuUfUf | | GTGTCCACAAAGTCA | |
| | UfcaaagcuauuL96 | | gacuUfuGfuggacs | | AAGCTATT | |
| | | | asc | | | |
| AD-1657760 | csusaacaUfgUfUf | | asAfsaagGfcAfAf | | TACTAACATGTTATT | |
| | AfuuugccuuuuL96 | | auaaCfaUfguuags | | TGCCTTTT | |
| | | | usa | | | |
| AD-1657776 | csusuuugAfaUfUf | | asAfsgauAfaUfGf | | GCCTTTTGAATTCTC | |
| | CfucauuaucuuL96 | | agaaUfuCfaaaags | | ATTATCTT | |
| | | | gsc | | | |
| AD-1657793 | gsusauugUfgGfAf | | asUfscugGfaAfAf | | TTGTATTGTGGAGTT | |
| | GfuuuuccagauL96 | | acucCfaCfaauacs | | TTCCAGAG | |
| | | | asa | | | |
| AD-1657811 | gsasggccGfuGfUf | | asAfsucaCfaUfGf | | CAGAGGCCGTGTGAC | |
| | GfacaugugauuL96 | | ucacAfcGfgccucs | | ATGTGATT | |
| | | | usg | | | |
| AD-1657822 | ascsauguGfaUfUf | | asAfsagaUfgAfUf | | TGACATGTGATTACA | |
| | AfcaucaucuuuL96 | | guaaUfcAfcaugus | | TCATCTTT | |
| | | | csa | | | |
| AD-1657834 | asuscaucUfuUfCf | | asAfsaugAfuGfUf | | ACATCATCTTTCTGA | |
| | UfgacaucauuuL96 | | cagaAfaGfaugaus | | CATCATTG | |
| | | | gsu | | | |
| AD-1657845 | gsascaucAfuUfGf | | asAfsuucCfaUfUf | | CTGACATCATTGTTA | |
| | UfuaauggaauuL96 | | aacaAfuGfaugucs | | ATGGAATG | |
| | | | asg | | | |

TABLE 4
Unmodified Oligonucleotide Sequences of INHBE Antisense Polynucleotide Agents

| | | | | | Start | End |
|---|---|---|---|---|---|---|
| | Unmodified | SEQ | | SEQ | Position | Position |
| Oligonucleotide | Antisense | ID | Target Sequence | ID | in NM_ | in NM_ |
| Name | Sequence 5′ to 3′ | NO: | 5′ to 3′ | NO: | 031479.5 | 031479.5 |
| ENST00000266646_1576 | TGCCTTCCCTCCCTCCCTC | | GGAGGGAGGGAGGGAAGG | | 1576 | 1595 |
| | C | | CA | | | |
| ENST00000266646_1255 | GCCACAGGCCTCCACCAC | | UGGUGGUGGAGGCCUGUG | | 1255 | 1274 |
| | CA | | GC | | | |
| ENST00000266646_466 | AGCCACACTCCCTGGCTG | | UACAGCCAGGGAGUGUGG | | 466 | 485 |
| | TA | | CU | | | |
| ENST00000266646_827 | TCGGTTGTCCAGTAACTG | | CACAGUUACUGGACAACC | | 827 | 846 |
| | TG | | GA | | | |
| ENST00000266646_1626 | CCCTCACTTGAGGACTTT | | AGAAAGUCCUCAAGUGAG | | 1626 | 1645 |
| | CT | | GG | | | |
| ENST00000266646_1000 | GTCCCGCCATCCCAGTTC | | AGGAACUGGGAUGGCGGG | | 1000 | 1019 |
| | CT | | AC | | | |
| ENST00000266646_1482 | GCTTTACCCATCTCCCAA | | UGUUGGGAGAUGGGUAAA | | 1482 | 1501 |
| | CA | | GC | | | |
| ENST00000266646_1648 | ACCATCTATCTGCTTCCT | | GGAGGAAGCAGAUAGAUG | | 1648 | 1667 |
| | CC | | GU | | | |
| ENST00000266646_1442 | GCCAGAGTCTCAGACAA | | UUCUUGUCUGAGACUCUG | | 1442 | 1461 |
| | GAA | | GC | | | |
| ENST00000266646_5 | CCTCACAGCTCATGTCTG | | GCCAGACAUGAGCUGUGA | | 5 | 24 |
| | GC | | GG | | | |
| ENST00000266646_1736 | TTGCCCATCTCCCTCTTG | | GUCAAGAGGGAGAUGGGC | | 1736 | 1755 |
| | AC | | AA | | | |
| ENST00000266646_1123 | GGCTTTGAGGAGGCTGAA | | UCUUCAGCCUCCUCAAAG | | 1123 | 1142 |
| | GA | | CC | | | |
| ENST00000266646_2390 | TGTCACACGGCCTCTGGA | | UUUCCAGAGGCCGUGUGA | | 2390 | 2409 |
| | AA | | CA | | | |
| ENST00000266646_269 | GTGCTCGCACCAGTGCCC | | GUGGGCACUGGUGCGAGC | | 269 | 288 |
| | AC | | AC | | | |
| ENST00000266646_293 | AGGGACACACAGACCCT | | GACAGGGUCUGUGUGUCC | | 293 | 312 |
| | GTC | | CU | | | |
| ENST00000266646_852 | TGTCCTGCTGTGTCCAAG | | CUCUUGGACACAGCAGGA | | 852 | 871 |
| | AG | | CA | | | |
| ENST00000266646_1906 | GACAGAGCGAGACTCGA | | AGAUCGAGUCUCGCUCUG | | 1906 | 1925 |
| | TCT | | UC | | | |
| ENST00000266646_773 | GTTTCAGGACACCAGACT | | GAAGUCUGGUGUCCUGAA | | 773 | 792 |
| | TC | | AC | | | |
| ENST00000266646_1606 | CATCTTGGGAGAGGCTAA | | ACUUAGCCUCUCCCAAGA | | 1606 | 1625 |
| | GT | | UG | | | |
| ENST00000266646_2235 | ACTGTTGCTCTGGACCAT | | UGAUGGUCCAGAGCAACA | | 2235 | 2254 |
| | CA | | GU | | | |
| ENST00000266646_1054 | AGGGCACTGCCCACTGCA | | ACUGCAGUGGGCAGUGCC | | 1054 | 1073 |
| | GT | | CU | | | |
| ENST00000266646_385 | GGTCAGGTGCAACCCATC | | UGGAUGGGUUGCACCUGA | | 385 | 404 |
| | CA | | CC | | | |
| ENST00000266646_1361 | GGGTGTGGATCAGGAATC | | CAGAUUCCUGAUCCACAC | | 1361 | 1380 |
| | TG | | CC | | | |
| ENST00000266646_1547 | GACATCTTCTCACAGGAC | | AAGUCCUGUGAGAAGAUG | | 1547 | 1566 |
| | TT | | UC | | | |
| ENST00000266646_905 | GGCCTGCTCCAGGCTCAT | | CAAUGAGCCUGGAGCAGG | | 905 | 924 |
| | TG | | CC | | | |
| ENST00000266646_522 | GCTGAAGTGGAGTCTGTG | | GUCACAGACUCCACUUCA | | 522 | 541 |
| | AC | | GC | | | |
| ENST00000266646_2270 | GGACCTCCCAGAAGCCTA | | UGUAGGCUUCUGGGAGGU | | 2270 | 2289 |
| | CA | | CC | | | |
| ENST00000266646_559 | AGGAGTGGACAGGTGAA | | CUUUUCACCUGUCCACUCC | | 559 | 578 |
| | AAG | | U | | | |
| ENST00000266646_790 | TCTGCAGTCTAGTTGCAG | | AACUGCAACUAGACUGCA | | 790 | 809 |
| | TT | | GA | | | |
| ENST00000266646_96 | ACCCTTGGCCAAGGGTAC | | CUGUACCCUUGGCCAAGG | | 96 | 115 |
| | AG | | GU | | | |
| ENST00000266646_2298 | CTTTGACTTTGTGGACAC | | GGGUGUCCACAAAGUCAA | | 2298 | 2317 |
| | CC | | AG | | | |
| ENST00000266646_1221 | GTCTTGACCACATTGCCA | | AAUGGCAAUGUGGUCAAG | | 1221 | 1240 |
| | TT | | AC | | | |
| ENST00000266646_172 | CAGTTGCTCCGTCTGTTG | | CUCAACAGACGGAGCAAC | | 172 | 191 |
| | AG | | UG | | | |
| ENST00000266646_1422 | AGTGCCCATTTGGGTCCC | | AUGGGACCCAAAUGGGCA | | 1422 | 1441 |
| | AT | | CU | | | |
| ENST00000266646_2057 | ACATGTTGAAACCTCGTT | | GAAACGAGGUUUCAACAU | | 2057 | 2076 |
| | TC | | GU | | | |
| ENST00000266646_1149 | GAGGTACTGGCAGGCCA | | CCUUGGCCUGCCAGUACC | | 1149 | 1168 |
| | AGG | | UC | | | |
| ENST00000266646_352 | GGCTAGCTCCAGCACCAG | | CUCUGGUGCUGGAGCUAG | | 352 | 371 |
| | AG | | CC | | | |
| ENST00000266646_75 | CACCTGTCTTCTATTGTCT | | CAGACAAUAGAAGACAGG | | 75 | 94 |
| | G | | UG | | | |
| ENST00000266646_2252 | CAGAGTACACTTGAAGA | | AGUUCUUCAAGUGUACUC | | 2252 | 2271 |
| | ACT | | UG | | | |
| ENST00000266646_1853 | TGCTTGTCTCCCAGTGGG | | GACCCACUGGGAGACAAG | | 1853 | 1872 |
| | TC | | CA | | | |
| ENST00000266646_585 | CGGGCATGGTACAGGTG | | CACCACCUGUACCAUGCCC | | 585 | 604 |
| | GTG | | G | | | |
| ENST00000266646_1323 | GCCCTGTGCCTGGGAAAC | | AAGUUUCCCAGGCACAGG | | 1323 | 1342 |
| | TT | | GC | | | |
| ENST00000266646_204 | GTGAATGGCCCTGGTTCA | | CCUGAACCAGGGCCAUUC | | 204 | 223 |
| | GG | | AC | | | |
| ENST00000266646_1719 | GACCTTCCCTTAAGGTAC | | AGGUACCUUAAGGGAAGG | | 1719 | 1738 |
| | CT | | UC | | | |
| ENST00000266646_239 | GCCAGAGCTGGACATCA | | CCCUGAUGUCCAGCUCUG | | 239 | 258 |
| | GGG | | GC | | | |
| ENST00000266646_1384 | ATTGCCAGGTGGTTGTTG | | CCCAACAACCACCUGGCAA | | 1384 | 1403 |
| | GG | | U | | | |
| ENST00000266646_1017 | TCGGGCTGCAGTATCCAG | | GACUGGAUACUGCAGCCC | | 1017 | 1036 |
| | TC | | GA | | | |
| ENST00000266646_113 | ACACCACTGCCACACCTA | | GGUAGGUGUGGCAGUGGU | | 113 | 132 |
| | CC | | GU | | | |
| ENST00000266646_1753 | CATCCTCCCTCAGCGCCT | | CAAGGCGCUGAGGGAGGA | | 1753 | 1772 |
| | TG | | UG | | | |
| ENST00000266646_2197 | ACTCACATTTGTTGATTTT | | GAAAAUCAACAAAUGUGA | | 2197 | 2216 |
| | C | | GU | | | |
| ENST00000266646_2441 | TACAAGCACACATTCCAT | | UAAUGGAAUGUGUGCUUG | | 2441 | 2460 |
| | TA | | UA | | | |
| ENST00000266646_1297 | GGTCTCTTCACTCCAAAG | | GGCUUUGGAGUGAAGAGA | | 1297 | 1316 |
| | CC | | CC | | | |
| ENST00000266646_1272 | TCCTCTTGCTAGCTGCAG | | GGCUGCAGCUAGCAAGAG | | 1272 | 1291 |
| | CC | | GA | | | |
| ENST00000266646_1694 | GCCCTTACCCTGGGCCAG | | GGCUGGCCCAGGGUAAGG | | 1694 | 1713 |
| | CC | | GC | | | |
| ENST00000266646_1786 | TTTCCTGACTCCTGTTTCT | | CAGAAACAGGAGUCAGGA | | 1786 | 1805 |
| | G | | AA | | | |
| ENST00000266646_684 | AGGAGAGTGCGGGACCC | | CAAGGGUCCCGCACUCUCC | | 684 | 703 |
| | TTG | | U | | | |
| ENST00000266646_1674 | TGCTTACCCTGCTTCAAG | | GGCUUGAAGCAGGGUAAG | | 1674 | 1693 |
| | CC | | CA | | | |
| ENST00000266646_646 | CCATCGGAAGATCCTCAA | | GCUUGAGGAUCUUCCGAU | | 646 | 665 |
| | GC | | GG | | | |
| ENST00000266646_712 | GCCCAGGTTGGTGATGTG | | ACCACAUCACCAACCUGG | | 712 | 731 |
| | GT | | GC | | | |
| ENST00000266646_2351 | AGATAATGAGAATTCAA | | CUUUUGAAUUCUCAUUAU | | 2351 | 2370 |
| | AAG | | CU | | | |
| ENST00000266646_2373 | AAAACTCCACAATACAAT | | AAAUUGUAUUGUGGAGUU | | 2373 | 2392 |
| | TT | | UU | | | |
| ENST00000266646_1197 | TCCAGGTAGAGGAGAGA | | CUCUCUCUCCUCUACCUGG | | 1197 | 1216 |
| | GAG | | A | | | |
| ENST00000266646_1175 | GCCTTCGGGCAGTAGGGA | | UGUCCCUACUGCCCGAAG | | 1175 | 1194 |
| | CA | | GC | | | |
| ENST00000266646_1340 | TGCCTCCAGTCACAGATG | | GGCAUCUGUGACUGGAGG | | 1340 | 1359 |
| | CC | | CA | | | |
| ENST00000266646_44 | GGCTGAAAGTAGATCATC | | CAGAUGAUCUACUUUCAG | | 44 | 63 |
| | TG | | CC | | | |
| ENST00000266646_1463 | ACATCAGCCAACCTGGAA | | UAUUCCAGGUUGGCUGAU | | 1463 | 1482 |
| | TA | | GU | | | |
| ENST00000266646_444 | CTCCGGAGGGCTCTGGTC | | CUGACCAGAGCCCUCCGG | | 444 | 463 |
| | AG | | AG | | | |
| ENST00000266646_1103 | CGGCAGAATGGAAAGAG | | UGCCUCUUUCCAUUCUGC | | 1103 | 1122 |
| | GCA | | CG | | | |
| ENST00000266646_2124 | ATCTCATCACTTCGGGAG | | GCCUCCCGAAGUGAUGAG | | 2124 | 2143 |
| | GC | | AU | | | |
| ENST00000266646_2096 | GTGGATCAAGAGGTCAA | | CUCUUGACCUCUUGAUCC | | 2096 | 2115 |
| | GAG | | AC | | | |
| ENST00000266646_665 | GGCGCCTCCTCCTTGGTC | | GGGACCAAGGAGGAGGCG | | 665 | 684 |
| | CC | | CC | | | |
| ENST00000266646_2333 | AGGCAAATAACATGTTAG | | UACUAACAUGUUAUUUGC | | 2333 | 2352 |
| | TA | | CU | | | |
| ENST00000266646_976 | GTCTACGTAATGGTCTCG | | GGCGAGACCAUUACGUAG | | 976 | 995 |
| | CC | | AC | | | |
| ENST00000266646_942 | GTCGCAGGCTCACAGGTG | | CCCACCUGUGAGCCUGCG | | 942 | 961 |
| | GG | | AC | | | |
| ENST00000266646_733 | GGGCAGAGTTAAGGTAT | | GGCAUACCUUAACUCUGC | | 733 | 752 |
| | GCC | | CC | | | |
| ENST00000266646_1817 | ACCAGGGAACTTCTTAGG | | AGCCUAAGAAGUUCCCUG | | 1817 | 1836 |
| | CT | | GU | | | |
| ENST00000266646_2161 | AGAAAGTATAAGCCAGG | | GCGCCUGGCUUAUACUUU | | 2161 | 2180 |
| | CGC | | CU | | | |
| ENST00000266646_496 | AAAGCTGATGACCTCCTC | | GGGAGGAGGUCAUCAGCU | | 496 | 515 |
| | CC | | UU | | | |
| ENST00000266646_604 | GAGCACGTGCAGCCACA | | GCCUGUGGCUGCACGUGC | | 604 | 623 |
| | GGC | | UC | | | |
| ENST00000266646_1872 | AGAAGAAAGAAAGTATA | | AUUUAUACUUUCUUUCUU | | 1872 | 1891 |
| | AAT | | CU | | | |
| ENST00000266646_1526 | GGGCAGGAAATCATGCTT | | GAAAGCAUGAUUUCCUGC | | 1526 | 1545 |
| | TC | | CC | | | |
| ENST00000266646_2217 | CACCCTAACCCTTCTTTA | | CAUAAAGAAGGGUUAGGG | | 2217 | 2236 |
| | TG | | UG | | | |
| ENST00000266646_1238 | CCATATCTGGCACATCCG | | GACGGAUGUGCCAGAUAU | | 1238 | 1257 |
| | TC | | GG | | | |
| ENST00000266646_922 | GGTCCTCCTCCTGGCCCG | | GCCGGGCCAGGAGGAGGA | | 922 | 941 |
| | GC | | CC | | | |
| ENST00000266646_1079 | TGCCTGGGCTGCCAGCCA | | CCUGGCUGGCAGCCCAGG | | 1079 | 1098 |
| | GG | | CA | | | |
| ENST00000266646_23 | TGGATAGCTGTGCTTGAC | | GGGUCAAGCACAGCUAUC | | 23 | 42 |
| | CC | | CA | | | |
| ENST00000266646_2022 | AATTAGTGGGCGCCTGTA | | AUUACAGGCGCCCACUAA | | 2022 | 2041 |
| | AT | | UU | | | |
| ENST00000266646_874 | AAGCTCTAGGAAGGGCT | | AGCAGCCCUUCCUAGAGC | | 874 | 893 |
| | GCT | | UU | | | |
| ENST00000266646_2414 | TGTCAGAAAGATGATGTA | | AUUACAUCAUCUUUCUGA | | 2414 | 2433 |
| | AT | | CA | | | |
| ENST00000266646_626 | AAAGAGTGCCAGGAAGG | | CACCCUUCCUGGCACUCUU | | 626 | 645 |
| | GTG | | U | | | |

TABLE 5
Modified Oligonucleotide Sequences of INHBE Antisense Polynucleotide Agents

| | | Antisense | |
|---|---|---|---|
| | | Oligonucleotide | SEQ |
| | | Name - Alnylam | ID |
| Oligonucleotide Name | Modified Oligonucleotide Sequences 5′ to 3′ | Designation | NO: |
| ENST00000266646_1576 | (Teos)(Geos)(m5Ceos)(m5Ceos)(Teos)dTs(m5dCs)(m5dCs)(m5dCs)dTs(m5dCs) | A-3176625.1 | |
| | (m5dCs)(m5dCs)dTs(m5dCs)(m5Ceos)(m5Ceos)(Teos)(m5Ceos)(m5Ceo) | | |
| ENST00000266646_1255 | (Geos)(m5Ceos)(m5Ceos)(Aeos)(m5Ceos)dAsdGsdGs(m5dCs)(m5dCs)dTs(m5dCs) | A-3176626.1 | |
| | (m5dCs)dAs(m5dCs)(m5Ceos)(Aeos)(m5Ceos)(m5Ceos)(Aeo) | | |
| ENST00000266646_466 | (Aeos)(Geos)(m5Ceos)(m5Ceos)(Aeos)(m5dCs)dAs(m5dCs)dTs(m5dCs)(m5dCs) | A-3176627.1 | |
| | (m5dCs)dTsdGsdGs(m5Ceos)(Teos)(Geos)(Teos)(Aeo) | | |
| ENST00000266646_827 | (Teos)(m5Ceos)(Geos)(Geos)(Teos)dTsdGsdTs(m5dCs)(m5dCs)dAsdGsdTsdAsdAs | A-3176628.1 | |
| | (m5Ceos)(Teos)(Geos)(Teos)(Geo) | | |
| ENST00000266646_1626 | (m5Ceos)(m5Ceos)(m5Ceos)(Teos)(m5Ceos)dAs(m5dCs)dTsdTsdGsdAsdGsdGsdAs | A-3176629.1 | |
| | (m5dCs)(Teos)(Teos)(Teos)(m5Ceos)(Teo) | | |
| ENST00000266646_1000 | (Geos)(Teos)(m5Ceos)(m5Ceos)(m5Ceos)dGs(m5dCs)(m5dCs)dAsdTs(m5dCs) | A-3176630.1 | |
| | (m5dCs)(m5dCs)dAsdGs(Teos)(Teos)(m5Ceos)(m5Ceos)(Teo) | | |
| ENST00000266646_1482 | (Geos)(m5Ceos)(Teos)(Teos)(Teos)dAs(m5dCs)(m5dCs)(m5dCs)dAsdTs(m5dCs)dTs | A-3176631.1 | |
| | (m5dCs)(m5dCs)(m5Ceos)(Aeos)(Aeos)(m5Ceos)(Aeo) | | |
| ENST00000266646_1648 | (Aeos)(m5Ceos)(m5Ceos)(Aeos)(Teos)(m5dCs)dTsdAsdTs(m5dCs)dTsdGs(m5dCs) | A-3176632.1 | |
| | dTsdTs(m5Ceos)(m5Ceos)(Teos)(m5Ceos)(m5Ceo) | | |
| ENST00000266646_1442 | (Geos)(m5Ceos)(m5Ceos)(Aeos)(Geos)dAsdGsdTs(m5dCs)dTs(m5dCs)dAsdGsdAs | A-3176633.1 | |
| | (m5dCs)(Aeos)(Aeos)(Geos)(Aeos)(Aeo) | | |
| ENST00000266646_5 | (m5Ceos)(m5Ceos)(Teos)(m5Ceos)(Aeos)(m5dCs)dAsdGs(m5dCs)dTs(m5dCs) | A-3176634.1 | |
| | dAsdTsdGsdTs(m5Ceos)(Teos)(Geos)(Geos)(m5Ceo) | | |
| ENST00000266646_1736 | (Teos)(Teos)(Geos)(m5Ceos)(m5Ceos)(m5dCs)dAsdTs(m5dCs)dTs(m5dCs)(m5dCs) | A-3176635.1 | |
| | (m5dCs)dTs(m5dCs)(Teos)(Teos)(Geos)(Aeos)(m5Ceo) | | |
| ENST00000266646_1123 | (Geos)(Geos)(m5Ceos)(Teos)(Teos)dTsdGsdAsdGsdGsdAsdGsdGs(m5dCs)dTs | A-3176636.1 | |
| | (Geos)(Aeos)(Aeos)(Geos)(Aeo) | | |
| ENST00000266646_2390 | (Teos)(Geos)(Teos)(m5Ceos)(Aeos)(m5dCs)dAs(m5dCs)dGsdGs(m5dCs)(m5dCs) | A-3176637.1 | |
| | dTs(m5dCs)dTs(Geos)(Geos)(Aeos)(Aeos)(Aeo) | | |
| ENST00000266646_269 | (Geos)(Teos)(Geos)(m5Ceos)(Teos)(m5dCs)dGs(m5dCs)dAs(m5dCs)(m5dCs) | A-3176638.1 | |
| | dAsdGsdTsdGs(m5Ceos)(m5Ceos)(m5Ceos)(Aeos)(m5Ceo) | | |
| ENST00000266646_293 | (Aeos)(Geos)(Geos)(Geos)(Aeos)(m5dCs)dAs(m5dCs)dAs(m5dCs)dAsdGsdAs | A-3176639.1 | |
| | (m5dCs)(m5dCs)(m5Ceos)(Teos)(Geos)(Teos)(m5Ceo) | | |
| ENST00000266646_852 | (Teos)(Geos)(Teos)(m5Ceos)(m5Ceos)dTsdGs(m5dCs)dTsdGsdTsdGsdTs(m5dCs) | A-3176640.1 | |
| | (m5dCs)(Aeos)(Aeos)(Geos)(Aeos)(Geo) | | |
| ENST00000266646_1906 | (Geos)(Aeos)(m5Ceos)(Aeos)(Geos)dAsdGs(m5dCs)dGsdAsdGsdAs(m5dCs)dTs | A-3176641.1 | |
| | (m5dCs)(Geos)(Aeos)(Teos)(m5Ceos)(Teo) | | |
| ENST00000266646_773 | (Geos)(Teos)(Teos)(Teos)(m5Ceos)dAsdGsdGsdAs(m5dCs)dAs(m5dCs)(m5dCs) | A-3176642.1 | |
| | dAsdGs(Aeos)(m5Ceos)(Teos)(Teos)(m5Ceo) | | |
| ENST00000266646_1606 | (m5Ceos)(Aeos)(Teos)(m5Ceos)(Teos)dTsdGsdGsdGsdAsdGsdAsdGsdGs(m5dCs) | A-3176643.1 | |
| | (Teos)(Aeos)(Aeos)(Geos)(Teo) | | |
| ENST00000266646_2235 | (Aeos)(m5Ceos)(Teos)(Geos)(Teos)dTsdGs(m5dCs)dTs(m5dCs)dTsdGsdGsdAs | A-3176644.1 | |
| | (m5dCs)(m5Ceos)(Aeos)(Teos)(m5Ceos)(Aeo) | | |
| ENST00000266646_1054 | (Aeos)(Geos)(Geos)(Geos)(m5Ceos)dAs(m5dCs)dTsdGs(m5dCs)(m5dCs)(m5dCs) | A-3176645.1 | |
| | dAs(m5dCs)dTs(Geos)(m5Ceos)(Aeos)(Geos)(Teo) | | |
| ENST00000266646_385 | (Geos)(Geos)(Teos)(m5Ceos)(Aeos)dGsdGsdTsdGs(m5dCs)dAsdAs(m5dCs)(m5dCs) | A-3176646.1 | |
| | (m5dCs)(Aeos)(Teos)(m5Ceos)(m5Ceos)(Aeo) | | |
| ENST00000266646_1361 | (Geos)(Geos)(Geos)(Teos)(Geos)dTsdGsdGsdAsdTs(m5dCs)dAsdGsdGsdAs(Aeos) | A-3176647.1 | |
| | (Teos)(m5Ceos)(Teos)(Geo) | | |
| ENST00000266646_1547 | (Geos)(Aeos)(m5Ceos)(Aeos)(Teos)(m5dCs)dTsdTs(m5dCs)dTs(m5dCs)dAs(m5dCs) | A-3176648.1 | |
| | dAsdGs(Geos)(Aeos)(m5Ceos)(Teos)(Teo) | | |
| ENST00000266646_905 | (Geos)(Geos)(m5Ceos)(m5Ceos)(Teos)dGs(m5dCs)dTs(m5dCs)(m5dCs)dAsdGsdGs | A-3176649.1 | |
| | (m5dCs)dTs(m5Ceos)(Aeos)(Teos)(Teos)(Geo) | | |
| ENST00000266646_522 | (Geos)(m5Ceos)(Teos)(Geos)(Aeos)dAsdGsdTsdGsdGsdAsdGsdTs(m5dCs)dTs | A-3176650.1 | |
| | (Geos)(Teos)(Geos)(Aeos)(m5Ceo) | | |
| ENST00000266646_2270 | (Geos)(Geos)(Aeos)(m5Ceos)(m5Ceos)dTs(m5dCs)(m5dCs)(m5dCs) | A-3176651.1 | |
| | dAsdGsdAsdAsdGs(m5dCs)(m5Ceos)(Teos)(Aeos)(m5Ceos)(Aeo) | | |
| ENST00000266646_559 | (Aeos)(Geos)(Geos)(Aeos)(Geos)dTsdGsdGsdAs(m5dCs)dAsdGsdGsdTsdGs(Aeos) | A-3176652.1 | |
| | (Aeos)(Aeos)(Aeos)(Geo) | | |
| ENST00000266646_790 | (Teos)(m5Ceos)(Teos)(Geos)(m5Ceos)dAsdGsdTs(m5dCs)dTsdAsdGsdTsdTsdGs | A-3176653.1 | |
| | (m5Ceos)(Aeos)(Geos)(Teos)(Teo) | | |
| ENST00000266646_96 | (Aeos)(m5Ceos)(m5Ceos)(m5Ceos)(Teos)dTsdGsdGs(m5dCs)(m5dCs) | A-3176654.1 | |
| | dAsdAsdGsdGsdGs(Teos)(Aeos)(m5Ceos)(Aeos)(Geo) | | |
| ENST00000266646_2298 | (m5Ceos)(Teos)(Teos)(Teos)(Geos)dAs(m5dCs)dTsdTsdTsdGsdTsdGsdGsdAs | A-3176655.1 | |
| | (m5Ceos)(Aeos)(m5Ceos)(m5Ceos)(m5Ceo) | | |
| ENST00000266646_1221 | (Geos)(Teos)(m5Ceos)(Teos)(Teos)dGsdAs(m5dCs)(m5dCs)dAs(m5dCs) | A-3176656.1 | |
| | dAsdTsdTsdGs(m5Ceos)(m5Ceos)(Aeos)(Teos)(Teo) | | |
| ENST00000266646_172 | (m5Ceos)(Aeos)(Geos)(Teos)(Teos)dGs(m5dCs)dTs(m5dCs)(m5dCs)dGsdTs(m5dCs) | A-3176657.1 | |
| | dTsdGs(Teos)(Teos)(Geos)(Aeos)(Geo) | | |
| ENST00000266646_1422 | (Aeos)(Geos)(Teos)(Geos)(m5Ceos)(m5dCs)(m5dCs)dAsdTsdTsdTsdGsdGsdGsdTs | A-3176658.1 | |
| | (m5Ceos)(m5Ceos)(m5Ceos)(Aeos)(Teo) | | |
| ENST00000266646_2057 | (Aeos)(m5Ceos)(Aeos)(Teos)(Geos)dTsdTsdGsdAsdAsdAs(m5dCs)(m5dCs)dTs | A-3176659.1 | |
| | (m5dCs)(Geos)(Teos)(Teos)(Teos)(m5Ceo) | | |
| ENST00000266646_1149 | (Geos)(Aeos)(Geos)(Geos)(Teos)dAs(m5dCs)dTsdGsdGs(m5dCs)dAsdGsdGs(m5dCs) | A-3176660.1 | |
| | (m5Ceos)(Aeos)(Aeos)(Geos)(Geo) | | |
| ENST00000266646_352 | (Geos)(Geos)(m5Ceos)(Teos)(Aeos)dGs(m5dCs)dTs(m5dCs)(m5dCs)dAsdGs(m5dCs) | A-3176661.1 | |
| | dAs(m5dCs)(m5Ceos)(Aeos)(Geos)(Aeos)(Geo) | | |
| ENST00000266646_75 | (m5Ceos)(Aeos)(m5Ceos)(m5Ceos)(Teos)dGsdTs(m5dCs)dTsdTs(m5dCs) | A-3176662.1 | |
| | dTsdAsdTsdTs(Geos)(Teos)(m5Ceos)(Teos)(Geo) | | |
| ENST00000266646_2252 | (m5Ceos)(Aeos)(Geos)(Aeos)(Geos)dTsdAs(m5dCs)dAs(m5dCs)dTsdTsdGsdAsdAs | A-3176663.1 | |
| | (Geos)(Aeos)(Aeos)(m5Ceos)(Teo) | | |
| ENST00000266646_1853 | (Teos)(Geos)(m5Ceos)(Teos)(Teos)dGsdTs(m5dCs)dTs(m5dCs)(m5dCs)(m5dCs) | A-3176664.1 | |
| | dAsdGsdTs(Geos)(Geos)(Geos)(Teos)(m5Ceo) | | |
| ENST00000266646_585 | (m5Ceos)(Geos)(Geos)(Geos)(m5Ceos)dAsdTsdGsdGsdTsdAs(m5dCs)dAsdGsdGs | A-3176665.1 | |
| | (Teos)(Geos)(Geos)(Teos)(Geo) | | |
| ENST00000266646_1323 | (Geos)(m5Ceos)(m5Ceos)(m5Ceos)(Teos)dGsdTsdGs(m5dCs)(m5dCs) | A-3176666.1 | |
| | dTsdGsdGsdGsdAs(Aeos)(Aeos)(m5Ceos)(Teos)(Teo) | | |
| ENST00000266646_204 | (Geos)(Teos)(Geos)(Aeos)(Aeos)dTsdGsdGs(m5dCs)(m5dCs)(m5dCs)dTsdGsdGsdTs | A-3176667.1 | |
| | (Teos)(m5Ceos)(Aeos)(Geos)(Geo) | | |
| ENST00000266646_1719 | (Geos)(Aeos)(m5Ceos)(m5Ceos)(Teos)dTs(m5dCs)(m5dCs)(m5dCs) | A-3176668.1 | |
| | dTsdTsdAsdAsdGsdGs(Teos)(Aeos)(m5Ceos)(m5Ceos)(Teo) | | |
| ENST00000266646_239 | (Geos)(m5Ceos)(m5Ceos)(Aeos)(Geos)dAsdGs(m5dCs)dTsdGsdGsdAs(m5dCs) | A-3176669.1 | |
| | dAsdTs(m5Ceos)(Aeos)(Geos)(Geos)(Geo) | | |
| ENST00000266646_1384 | (Aeos)(Teos)(Teos)(Geos)(m5Ceos)(m5dCs)dAsdGsdGsdTsdGsdGsdTsdTsdGs(Teos) | A-3176670.1 | |
| | (Teos)(Geos)(Geos)(Geo) | | |
| ENST00000266646_1017 | (Teos)(m5Ceos)(Geos)(Geos)(Geos)(m5dCs)dTsdGs(m5dCs)dAsdGsdTsdAsdTs | A-3176671.1 | |
| | (m5dCs)(m5Ceos)(Aeos)(Geos)(Teos)(m5Ceo) | | |
| ENST00000266646_113 | (Aeos)(m5Ceos)(Aeos)(m5Ceos)(m5Ceos)dAs(m5dCs)dTsdGs(m5dCs)(m5dCs)dAs | A-3176672.1 | |
| | (m5dCs)dAs(m5dCs)(m5Ceos)(Teos)(Aeos)(m5Ceos)(m5Ceo) | | |
| ENST00000266646_1753 | (m5Ceos)(Aeos)(Teos)(m5Ceos)(m5Ceos)dTs(m5dCs)(m5dCs)(m5dCs)dTs(m5dCs) | A-3176673.1 | |
| | dAsdGs(m5dCs)dGs(m5Ceos)(m5Ceos)(Teos)(Teos)(Geo) | | |
| ENST00000266646_2197 | (Aeos)(m5Ceos)(Teos)(m5Ceos)(Aeos)(m5dCs)dAsdTsdTsdTsdGsdTsdTsdGsdAs | A-3176674.1 | |
| | (Teos)(Teos)(Teos)(Teos)(m5Ceo) | | |
| ENST00000266646_2441 | (Teos)(Aeos)(m5Ceos)(Aeos)(Aeos)dGs(m5dCs)dAs(m5dCs)dAs(m5dCs)dAsdTsdTs | A-3176675.1 | |
| | (m5dCs)(m5Ceos)(Aeos)(Teos)(Teos)(Aeo) | | |
| ENST00000266646_1297 | (Geos)(Geos)(Teos)(m5Ceos)(Teos)(m5dCs)dTsdTs(m5dCs)dAs(m5dCs)dTs(m5dCs) | A-3176676.1 | |
| | (m5dCs)dAs(Aeos)(Aeos)(Geos)(m5Ceos)(m5Ceo) | | |
| ENST00000266646_1272 | (Teos)(m5Ceos)(m5Ceos)(Teos)(m5Ceos)dTsdTsdGs(m5dCs)dTsdAsdGs(m5dCs) | A-3176677.1 | |
| | dTsdGs(m5Ceos)(Aeos)(Geos)(m5Ceos)(m5Ceo) | | |
| ENST00000266646_1694 | (Geos)(m5Ceos)(m5Ceos)(m5Ceos)(Teos)dTsdAs(m5dCs)(m5dCs)(m5dCs) | A-3176678.1 | |
| | dTsdGsdGsdGs(m5dCs)(m5Ceos)(Aeos)(Geos)(m5Ceos)(m5Ceo) | | |
| ENST00000266646_1786 | (Teos)(Teos)(Teos)(m5Ceos)(m5Ceos)dTsdGsdAs(m5dCs)dTs(m5dCs)(m5dCs) | A-3176679.1 | |
| | dTsdGsdTs(Teos)(Teos)(m5Ceos)(Teos)(Geo) | | |
| ENST00000266646_684 | (Aeos)(Geos)(Geos)(Aeos)(Geos)dAsdGsdTsdGs(m5dCs)dGsdGsdGsdAs(m5dCs) | A-3176680.1 | |
| | (m5Ceos)(m5Ceos)(Teos)(Teos)(Geo) | | |
| ENST00000266646_1674 | (Teos)(Geos)(m5Ceos)(Teos)(Teos)dAs(m5dCs)(m5dCs)(m5dCs)dTsdGs(m5dCs) | A-3176681.1 | |
| | dTsdTs(m5dCs)(Aeos)(Aeos)(Geos)(m5Ceos)(m5Ceo) | | |
| ENST00000266646_646 | (m5Ceos)(m5Ceos)(Aeos)(Teos)(m5Ceos)dGsdGsdAsdAsdGsdAsdTs(m5dCs)(m5dCs) | A-3176682.1 | |
| | dTs(m5Ceos)(Aeos)(Aeos)(Geos)(m5Ceo) | | |
| ENST00000266646_712 | (Geos)(m5Ceos)(m5Ceos)(m5Ceos)(Aeos)dGsdGsdTsdTsdGsdGsdTsdGsdAsdTs(Geos) | A-3176683.1 | |
| | (Teos)(Geos)(Geos)(Teo) | | |
| ENST00000266646_2351 | (Aeos)(Geos)(Aeos)(Teos)(Aeos)dAsdTsdGsdAsdGsdAsdAsdTsdTs(m5dCs)(Aeos) | A-3176684.1 | |
| | (Aeos)(Aeos)(Aeos)(Geo) | | |
| ENST00000266646_2373 | (Aeos)(Aeos)(Aeos)(Aeos)(m5Ceos)dTs(m5dCs)(m5dCs)dAs(m5dCs)dAsdAsdTsdAs | A-3176685.1 | |
| | (m5dCs)(Aeos)(Aeos)(Teos)(Teos)(Teo) | | |
| ENST00000266646_1197 | (Teos)(m5Ceos)(m5Ceos)(Aeos)(Geos)dGsdTsdAsdGsdAsdGsdGsdAsdGsdAs(Geos) | A-3176686.1 | |
| | (Aeos)(Geos)(Aeos)(Geo) | | |
| ENST00000266646_1175 | (Geos)(m5Ceos)(m5Ceos)(Teos)(Teos)(m5dCs)dGsdGsdGs(m5dCs) | A-3176687.1 | |
| | dAsdGsdTsdAsdGs(Geos)(Geos)(Aeos)(m5Ceos)(Aeo) | | |
| ENST00000266646_1340 | (Teos)(Geos)(m5Ceos)(m5Ceos)(Teos)(m5dCs)(m5dCs)dAsdGsdTs(m5dCs)dAs | A-3176688.1 | |
| | (m5dCs)dAsdGs(Aeos)(Teos)(Geos)(m5Ceos)(m5Ceo) | | |
| ENST00000266646_44 | (Geos)(Geos)(m5Ceos)(Teos)(Geos)dAsdAsdAsdGsdTsdAsdGsdAsdTs(m5dCs)(Aeos) | A-3176689.1 | |
| | (Teos)(m5Ceos)(Teos)(Geo) | | |
| ENST00000266646_1463 | (Aeos)(m5Ceos)(Aeos)(Teos)(m5Ceos)dAsdGs(m5dCs)(m5dCs)dAsdAs(m5dCs) | A-3176690.1 | |
| | (m5dCs)dTsdGs(Geos)(Aeos)(Aeos)(Teos)(Aeo) | | |
| ENST00000266646_444 | (m5Ceos)(Teos)(m5Ceos)(m5Ceos)(Geos)dGsdAsdGsdGsdGs(m5dCs)dTs(m5dCs) | A-3176691.1 | |
| | dTsdGs(Geos)(Teos)(m5Ceos)(Aeos)(Geo) | | |
| ENST00000266646_1103 | (m5Ceos)(Geos)(Geos)(m5Ceos)(Aeos)dGsdAsdAsdTsdGsdGsdAsdAsdAsdGs(Aeos) | A-3176692.1 | |
| | (Geos)(Geos)(m5Ceos)(Aeo) | | |
| ENST00000266646_2124 | (Aeos)(Teos)(m5Ceos)(Teos)(m5Ceos)dAsdTs(m5dCs)dAs(m5dCs)dTsdTs(m5dCs) | A-3176693.1 | |
| | dGsdGs(Geos)(Aeos)(Geos)(Geos)(m5Ceo) | | |
| ENST00000266646_2096 | (Geos)(Teos)(Geos)(Geos)(Aeos)dTs(m5dCs)dAsdAsdGsdAsdGsdGsdTs(m5dCs) | A-3176694.1 | |
| | (Aeos)(Aeos)(Geos)(Aeos)(Geo) | | |
| ENST00000266646_665 | (Geos)(Geos)(m5Ceos)(Geos)(m5Ceos)(m5dCs)dTs(m5dCs)(m5dCs)dTs(m5dCs) | A-3176695.1 | |
| | (m5dCs)dTsdTsdGs(Geos)(Teos)(m5Ceos)(m5Ceos)(m5Ceo) | | |
| ENST00000266646_2333 | (Aeos)(Geos)(Geos)(m5Ceos)(Aeos)dAsdAsdTsdAsdAs(m5dCs)dAsdTsdGsdTs(Teos) | A-3176696.1 | |
| | (Aeos)(Geos)(Teos)(Aeo) | | |
| ENST00000266646_976 | (Geos)(Teos)(m5Ceos)(Teos)(Aeos)(m5dCs)dGsdTsdAsdAsdTsdGsdGsdTs(m5dCs) | A-3176697.1 | |
| | (Teos)(m5Ceos)(Geos)(m5Ceos)(m5Ceo) | | |
| ENST00000266646_942 | (Geos)(Teos)(m5Ceos)(Geos)(m5Ceos)dAsdGsdGs(m5dCs)dTs(m5dCs)dAs(m5dCs) | A-3176698.1 | |
| | dAsdGs(Geos)(Teos)(Geos)(Geos)(Geo) | | |
| ENST00000266646_733 | (Geos)(Geos)(Geos)(m5Ceos)(Aeos)dGsdAsdGsdTsdTsdAsdAsdGsdGsdTs(Aeos) | A-3176699.1 | |
| | (Teos)(Geos)(m5Ceos)(m5Ceo) | | |
| ENST00000266646_1817 | (Aeos)(m5Ceos)(m5Ceos)(Aeos)(Geos)dGsdGsdAsdAs(m5dCs)dTsdTs(m5dCs) | A-3176700.1 | |
| | dTsdTs(Aeos)(Geos)(Geos)(m5Ceos)(Teo) | | |
| ENST00000266646_2161 | (Aeos)(Geos)(Aeos)(Aeos)(Aeos)dGsdTsdAsdTsdAsdAsdGs(m5dCs)(m5dCs)dAs | A-3176701.1 | |
| | (Geos)(Geos)(m5Ceos)(Geos)(m5Ceo) | | |
| ENST00000266646_496 | (Aeos)(Aeos)(Aeos)(Geos)(m5Ceos)dTsdGsdAsdTsdGsdAs(m5dCs)(m5dCs)dTs | A-3176702.1 | |
| | (m5dCs)(m5Ceos)(Teos)(m5Ceos)(m5Ceos)(m5Ceo) | | |
| ENST00000266646_604 | (Geos)(Aeos)(Geos)(m5Ceos)(Aeos)(m5dCs)dGsdTsdGs(m5dCs)dAsdGs(m5dCs) | A-3176703.1 | |
| | (m5dCs)dAs(m5Ceos)(Aeos)(Geos)(Geos)(m5Ceo) | | |
| ENST00000266646_1872 | (Aeos)(Geos)(Aeos)(Aeos)(Geos)dAsdAsdAsdGsdAsdAsdAsdGsdTsdAs(Teos)(Aeos) | A-3176704.1 | |
| | (Aeos)(Aeos)(Teo) | | |
| ENST00000266646_1526 | (Geos)(Geos)(Geos)(m5Ceos)(Aeos)dGsdGsdAsdAsdAsdTs(m5dCs)dAsdTsdGs | A-3176705.1 | |
| | (m5Ceos)(Teos)(Teos)(Teos)(m5Ceo) | | |
| ENST00000266646_2217 | (m5Ceos)(Aeos)(m5Ceos)(m5Ceos)(m5Ceos)dTsdAsdAs(m5dCs)(m5dCs)(m5dCs) | A-3176706.1 | |
| | dTsdTs(m5dCs)dTs(Teos)(Teos)(Aeos)(Teos)(Geo) | | |
| ENST00000266646_1238 | (m5Ceos)(m5Ceos)(Aeos)(Teos)(Aeos)dTs(m5dCs)dTsdGsdGs(m5dCs)dAs(m5dCs) | A-3176707.1 | |
| | dAsdTs(m5Ceos)(m5Ceos)(Geos)(Teos)(m5Ceo) | | |
| ENST00000266646_922 | (Geos)(Geos)(Teos)(m5Ceos)(m5Ceos)dTs(m5dCs)(m5dCs)dTs(m5dCs)(m5dCs) | A-3176708.1 | |
| | dTsdGsdGs(m5dCs)(m5Ceos)(m5Ceos)(Geos)(Geos)(m5Ceo) | | |
| ENST00000266646_1079 | (Teos)(Geos)(m5Ceos)(m5Ceos)(Teos)dGsdGsdGs(m5dCs)dTsdGs(m5dCs)(m5dCs) | A-3176709.1 | |
| | dAsdGs(m5Ceos)(m5Ceos)(Aeos)(Geos)(Geo) | | |
| ENST00000266646_23 | (Teos)(Geos)(Geos)(Aeos)(Teos)dAsdGs(m5dCs)dTsdGsdTsdGs(m5dCs)dTsdTs(Geos) | A-3176710.1 | |
| | (Aeos)(m5Ceos)(m5Ceos)(m5Ceo) | | |
| ENST00000266646_2022 | (Aeos)(Aeos)(Teos)(Teos)(Aeos)dGsdTsdGsdGsdGs(m5dCs)dGs(m5dCs)(m5dCs)dTs | A-3176711.1 | |
| | (Geos)(Teos)(Aeos)(Aeos)(Teo) | | |
| ENST00000266646_874 | (Aeos)(Aeos)(Geos)(m5Ceos)(Teos)(m5dCs)dTsdAsdGsdGsdAsdAsdGsdGsdGs | A-3176712.1 | |
| | (m5Ceos)(Teos)(Geos)(m5Ceos)(Teo) | | |
| ENST00000266646_2414 | (Teos)(Geos)(Teos)(m5Ceos)(Aeos)dGsdAsdAsdAsdGsdAsdTsdGsdAsdTs(Geos) | A-3176713.1 | |
| | (Teos)(Aeos)(Aeos)(Teo) | | |
| ENST00000266646_626 | (Aeos)(Aeos)(Aeos)(Geos)(Aeos)dGsdTsdGs(m5dCs)(m5dCs)dAsdGsdGsdAsdAs | A-3176714.1 | |
| | (Geos)(Geos)(Geos)(Teos)(Geo) | | |

TABLE 6
Single-Dose ASO Screen for INHBE in Hep3B cells

| Antisense | INHBE/gapdh | | INHBE/gapdh | |
|---|---|---|---|---|
| Oligonucleotide Name - | 10 nM | | 1 nM | |
| Alnylam Designation | mean | SD | mean | SD |
| A-3176625.1 | 51.192 | 0.603 | 93.091 | 1.689 |
| A-3176626.1 | 8.059 | 1.115 | 74.766 | 2.605 |
| A-3176627.1 | 27.184 | 2.748 | 104.884 | 7.820 |
| A-3176628.1 | 26.400 | 5.420 | 99.186 | 3.441 |
| A-3176629.1 | 21.365 | 4.263 | 92.178 | 4.841 |
| A-3176630.1 | 18.348 | 4.143 | 85.238 | 3.977 |
| A-3176631.1 | 31.065 | 2.242 | 82.673 | 5.501 |
| A-3176632.1 | 21.348 | 3.882 | 91.270 | 19.564 |
| A-3176633.1 | 23.428 | 0.452 | 85.927 | 1.955 |
| A-3176634.1 | 58.174 | 2.250 | 86.680 | 14.990 |
| A-3176635.1 | 30.935 | 1.635 | 107.367 | 12.928 |
| A-3176636.1 | 30.134 | 2.879 | 108.501 | 12.691 |
| A-3176637.1 | 41.079 | 1.351 | 110.259 | 4.245 |
| A-3176638.1 | 45.650 | 1.952 | 104.804 | 5.896 |
| A-3176639.1 | 49.022 | 2.092 | 110.324 | 11.820 |
| A-3176640.1 | 36.498 | 3.768 | 101.611 | 4.682 |
| A-3176641.1 | 18.946 | 3.360 | 91.703 | 3.943 |
| A-3176642.1 | 43.043 | 2.396 | 102.505 | 13.448 |
| A-3176643.1 | 58.410 | 4.227 | 114.161 | 17.325 |
| A-3176644.1 | 22.435 | 2.392 | 85.204 | 4.508 |
| A-3176645.1 | 40.724 | 3.082 | 98.422 | 13.411 |
| A-3176646.1 | 34.737 | 3.564 | 87.302 | 6.244 |
| A-3176647.1 | 47.141 | 1.009 | 92.879 | 6.005 |
| A-3176648.1 | 47.229 | 2.001 | 80.127 | 19.028 |
| A-3176649.1 | 103.484 | 4.492 | 95.801 | 3.731 |
| A-3176650.1 | 62.530 | 4.346 | 103.113 | 21.362 |
| A-3176651.1 | 33.491 | 1.963 | 97.878 | 33.861 |
| A-3176652.1 | 46.682 | 2.328 | 90.505 | 5.212 |
| A-3176653.1 | 46.690 | 3.856 | 95.193 | 2.577 |
| A-3176654.1 | 81.803 | 2.870 | 96.571 | 4.973 |
| A-3176655.1 | 23.367 | 2.439 | 89.955 | 3.694 |
| A-3176656.1 | 50.621 | 4.052 | 96.567 | 5.424 |
| A-3176657.1 | 43.979 | 2.624 | 98.563 | 1.943 |
| A-3176658.1 | 33.114 | 2.710 | 88.088 | 4.503 |
| A-3176659.1 | 28.785 | 1.812 | 89.803 | 2.435 |
| A-3176660.1 | 28.643 | 1.121 | 92.195 | 6.855 |
| A-3176661.1 | 31.450 | 1.403 | 88.997 | 3.281 |
| A-3176662.1 | 55.488 | 4.793 | 117.712 | 32.655 |
| A-3176663.1 | 41.608 | 2.154 | 93.474 | 5.595 |
| A-3176664.1 | 31.959 | 0.722 | 80.745 | 1.631 |
| A-3176665.1 | 58.038 | 4.692 | 97.514 | 2.907 |
| A-3176666.1 | 44.189 | 3.593 | 89.228 | 8.710 |
| A-3176667.1 | 80.541 | 3.170 | 97.269 | 5.151 |
| A-3176668.1 | 39.789 | 0.976 | 79.359 | 2.412 |
| A-3176669.1 | 49.085 | 3.293 | 89.408 | 5.140 |
| A-3176670.1 | 90.673 | 9.767 | 98.517 | 7.050 |
| A-3176671.1 | 40.467 | 2.213 | 95.310 | 71.768 |
| A-3176672.1 | 56.793 | 1.077 | 93.645 | 4.505 |
| A-3176673.1 | 37.695 | 1.452 | 72.950 | 14.812 |
| A-3176674.1 | 41.638 | 1.309 | 89.725 | 6.216 |
| A-3176675.1 | 53.549 | 3.826 | 108.649 | 25.494 |
| A-3176676.1 | 36.859 | 0.931 | 79.435 | 17.412 |
| A-3176677.1 | 39.240 | 5.511 | 86.177 | 8.146 |
| A-3176678.1 | 37.041 | 1.262 | 80.683 | 4.706 |
| A-3176679.1 | 47.681 | 2.196 | 95.029 | 17.729 |
| A-3176680.1 | 63.520 | 4.117 | 84.868 | 6.457 |
| A-3176681.1 | 48.092 | 4.264 | 86.458 | 27.868 |
| A-3176682.1 | 60.122 | 1.450 | 89.746 | 6.488 |
| A-3176683.1 | 52.770 | 2.661 | 79.356 | 4.009 |
| A-3176684.1 | 82.456 | 5.211 | 89.499 | 10.451 |
| A-3176685.1 | 64.430 | 1.923 | 97.314 | 1.501 |
| A-3176686.1 | 91.745 | 6.663 | 104.301 | 5.023 |
| A-3176687.1 | 63.608 | 6.412 | 96.022 | 9.206 |
| A-3176688.1 | 44.602 | 1.763 | 97.443 | 5.151 |
| A-3176689.1 | 79.137 | 3.742 | 101.440 | 11.939 |
| A-3176690.1 | 56.697 | 3.146 | 97.405 | 4.025 |
| A-3176691.1 | 55.203 | 3.092 | 106.389 | 15.186 |
| A-3176692.1 | 65.034 | 3.992 | 102.017 | 9.029 |
| A-3176693.1 | 24.934 | 1.017 | 71.277 | 3.138 |
| A-3176694.1 | 36.078 | 1.241 | 90.845 | 5.112 |
| A-3176695.1 | 66.832 | 1.621 | 95.640 | 2.202 |
| A-3176696.1 | 54.558 | 1.600 | 104.647 | 10.679 |
| A-3176697.1 | 48.591 | 1.822 | 96.709 | 9.787 |
| A-3176698.1 | 91.765 | 4.469 | 98.363 | 4.710 |
| A-3176699.1 | 66.474 | 2.024 | 102.313 | 7.744 |
| A-3176700.1 | 48.645 | 2.508 | 94.161 | 8.743 |
| A-3176701.1 | 47.411 | 0.925 | 100.947 | 4.900 |
| A-3176702.1 | 51.405 | 2.276 | 103.520 | 7.957 |
| A-3176703.1 | 40.230 | 1.318 | 92.059 | 2.618 |
| A-3176704.1 | 104.175 | 6.898 | 98.811 | 7.772 |
| A-3176705.1 | 39.979 | 2.464 | 90.747 | 8.034 |
| A-3176706.1 | 68.163 | 2.909 | 104.205 | 5.295 |
| A-3176707.1 | 45.798 | 1.461 | 92.025 | 5.975 |
| A-3176708.1 | 40.917 | 1.475 | 91.428 | 5.292 |
| A-3176709.1 | 38.277 | 4.085 | 95.297 | 2.152 |
| A-3176710.1 | 55.263 | 2.906 | 96.373 | 9.721 |
| A-3176711.1 | 41.687 | 0.437 | 102.038 | 4.486 |
| A-3176712.1 | 46.838 | 1.023 | 95.795 | 6.415 |
| A-3176713.1 | 48.598 | 1.431 | 98.072 | 7.452 |
| A-3176714.1 | 56.078 | 1.668 | 98.996 | 6.793 |

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A modulator of inhibin subunit beta E (INHBE).

2. The modulator of claim 1, wherein the modulator is selected from the group consisting of an oligonucleotide that targets INHBE; an antibody, or antigen-binding fragment thereof, that specifically binds INHBE; a small molecule; a guideRNA that effects ADAR editing; and a guideRNA that effects CRISPR editing.

3. The modulator of claim 2, wherein the oligonucleotide that targets INHBE is a double stranded ribonucleic acid (dsRNA) or an antisense polynucleotide agent.

4.-10. (canceled)

11. The modulator of claim 3, wherein the antisense polynucleotide agent comprises 4 to 50 contiguous nucleotides, wherein at least one of the contiguous nucleotides is a modified nucleotide, and wherein the nucleotide sequence of the agent is 80% complementary over its entire length to the equivalent region of the nucleotide sequence of any one of SEQ ID NOs:1, 2, 4, 6, 8, or 10.

12. The modulator of claim 11, wherein the equivalent region is any one of the target regions of SEQ ID NO:1 provided in Table 4.

13. The modulator of claim 3, wherein the antisense polynucleotide agent comprises at least 8 contiguous nucleotides differing by no more than 3 nucleotides from any one of the nucleotide sequences listed in Table 3.

14. (canceled)

15. The modulator of claim 3, wherein all of the nucleotides of the antisense polynucleotide agent comprise a nucleotide modification.

16. The modulator of claim 3, wherein the antisense polynucleotide agent is 10 to 40 nucleotides in length: 10-30 nucleotides in length, 18-30 nucleotides in length; or 18-24 nucleotides in length.

17.-23. (canceled)

24. The modulator of claim 3, wherein the modified nucleotide comprises a modified sugar moiety selected from the group consisting of a 2′-O-methoxyethyl modified sugar moiety, a 2′-methoxy modified sugar moiety, a 2′-O-alkyl modified sugar moiety, and a bicyclic sugar moiety.

25. (canceled)

26. The modulator of claim 3, wherein the modified nucleotide is a 5-methylcytosine.

27. The modulator of claim 3, wherein the modified nucleotide comprises a modified internucleoside linkage.

28.-43. (canceled)

44. The modulator of claim 3, wherein the antisense polynucleotide agent comprises
a gap segment consisting of linked deoxynucleotides;
a 5′-wing segment consisting of linked nucleotides;
a 3′-wing segment consisting of linked nucleotides;
wherein the gap segment is positioned between the 5′-wing segment and the 3′-wing segment and wherein each nucleotide of each wing segment comprises a modified sugar.

45. The modulator of claim 44, wherein the gap segment is ten 2′-deoxynucleotides in length and each of the wing segments is five nucleotides in length.

46.-50. (canceled)

51. The modulator of claim 3, wherein the modulator further comprises a ligand.

52.-54. (canceled)

55. A pharmaceutical composition for inhibiting expression and/or activity of INHBE comprising the modulator of claim 1.

56.-63. (canceled)

64. A method of inhibiting the expression and/or activity of INHBE in a cell, the method comprising:
(a) contacting the cell with a modulator of claim 1; and
(b) maintaining the cell produced in step (a) for a time sufficient to obtain inhibition of INHBE expression and/or activity, thereby inhibiting expression and/or activity of INHBE in the cell.

65.-72. (canceled)

73. A method of treating a subject having a disorder that would benefit from reduction in inhibin subunit beta E (INHBE) expression and/or activity, comprising administering to the subject a therapeutically effective amount of the modulator of claim 1, thereby treating the subject having the disorder that would benefit from reduction in INHBE expression.

74. (canceled)

75. The method of claim 73, wherein the disorder is an INHBE-associated disorder.

76. The method of claim 75, wherein the INHBE-associated disorder is selected from the group consisting of a metabolic disorder, a cardiovascular disorder, and hypertension.

77.-85. (canceled)

86. A kit, a vial, or a syringe comprising the modulator of claim 1.

87. (canceled)

88. (canceled)
